(19) United States
(12) Patent Application Publication
(10) Pub. No.: US 2013/0252821 A1
(43) Pub. Date: Sep. 26, 2013

(54) METHODS AND COMPOSITIONS FOR THE TREATMENT AND DIAGNOSIS OF DISEASES CHARACTERIZED BY VASCULAR LEAK, HYPOTENSION, OR A PROCOAGULANT STATE

(71) Applicants: Vikas P. Sukhatme, Newton, MA (US); S. Ananth Karumanchi, Chestnut Hill, MA (US); Samir M. Parikh, Roslindale, MA (US)

(72) Inventors: Vikas P. Sukhatme, Newton, MA (US); S. Ananth Karumanchi, Chestnut Hill, MA (US); Samir M. Parikh, Roslindale, MA (US)

(73) Assignee: Beth Israel Deaconess Medical Center, Inc., Boston, MA (US)

(21) Appl. No.: 13/608,606

(22) Filed: Sep. 10, 2012

Related U.S. Application Data
(63) Continuation of application No. 12/359,954, filed on Jan. 26, 2009, now abandoned.
Application No. 12/359,954 is a continuation of application No. 11/519,954, filed on Sep. 12, 2006, now abandoned.
(60) Provisional application No. 60/798,639, filed on May 8, 2006.
(60) Provisional application No. 60/716,339, filed on Sep. 12, 2005.

(51) Int. Cl. C12Q 1/68

(52) U.S. Cl. CPC C12Q 1/6883
USPC 506/2; 435/6.12; 435/6.11; 506/9

(57) ABSTRACT

Disclosed herein are methods for treating a vascular leak disorder, hypotension, or a procoagulant state using angiopoietin-2 (Ang-2) antagonist compounds. Also disclosed are methods for treating a vascular leak disorder associated with high dose IL-2 therapy using angiopoietin-2 antagonist compounds. Methods for diagnosing and monitoring vascular leak disorders, hypotension, or a procoagulant state that include the measurement of Ang-2 polypeptide or nucleic acid levels are also disclosed. Methods for inducing a vascular leak using an Ang-2 agonist are also disclosed.

### CROSS-REFERENCE TO RELATED APPLICATIONS

This application is a continuation of U.S. Utility application Ser. No. 12/359,954, filed Jan. 26, 2009, which is a continuation of U.S. Utility application Ser. No. 11/519,954, filed Sep. 12, 2006, which claims the benefit of the filing date of U.S. provisional application Nos. 60/798,639, filed on May 8, 2006, and 60/716,339, filed on Sep. 12, 2005, each of which is herein incorporated by reference.

### BACKGROUND OF THE INVENTION

In general, the invention relates to methods and compositions for the treatment and diagnosis of diseases characterized by vascular leak, hypotension, or a procoagulant state.

Blood vessels are normally lined with tightly linked cells, called endothelial cells, that form an impermeable barrier. Vascular leak occurs when small blood vessels, generally a capillary or venule, become leaky and release fluid. Vascular leak can occur under a variety of conditions, including sepsis, and can affect almost all the organ beds. The most serious effects of vascular leak include a drop in blood pressure, severe organ damage, and a lack of oxygenation of the blood when the leak is in the lung.

Sepsis is defined by the occurrence of systemic inflammation in the context of infection. It accounts for 3% of all admissions to U.S. hospitals, generates annual direct costs in excess of $16 billion, and is associated with an acute mortality of ˜30%. Death in sepsis is due to septic shock and multi-organ dysfunction. In sepsis, capillary permeability, which is a tightly regulated feature of microcirculation in all organ beds, is fundamentally altered, resulting in net extravasation of fluid out of the vascular space and into tissues. A dramatic manifestation of this phenomenon is acute lung injury (ALI) and its sequela, acute respiratory distress syndrome (ARDS), which occurs in up to 40% of patients with sepsis. ARDS is marked by leakage of fluid out of pulmonary capillaries and into alveolar septa and air spaces. Excess extravascular fluid in the lung impairs gas exchange across the alveolar membrane and decreases lung compliance. In ARDS, small blood vessels in the lungs become leaky and release fluid resulting in impaired lung function. Often, the damage becomes extensive enough to necessitate the use of mechanical ventilation. If the condition lasts too long, the lung tissue gets damaged irreversibly and ARDS associated with sepsis has been correlated with a mortality of 40%.

Vascular leak has been difficult to assess because there are no widely applicable tools to measure this process. In addition, in part due to the extensive heterogeneity in the patient population, there is no known cure or widely effective therapy for ARDS. In fact, mechanical ventilation remains the only proven mortality-improving intervention in ARDS. In addition, although survival has improved over the last thirty years, largely due to timely and multifaceted supportive care, outcomes in sepsis remain poor. Methods for diagnosing and treating diseases, such as ARDS, that are characterized by vascular leak, hypotension, or a procoagulant state are needed in the art. In addition, because ALI and ARDS, describe defects associated with numerous illnesses and not a single pathological entity, new markers are desperately needed to stratify these heterogeneous patients for future therapeutic trials.

### SUMMARY OF THE INVENTION

Angiopoietin-1 (Ang-1) and angiopoietin-2 (Ang-2), originally described as mediators of developmental angiogenesis are peptide ligands that bind the Tie-2 receptor tyrosine kinase found primarily on endothelial cells (ECs). Ang-1 and Ang-2 are thought to function as a competitive agonist/antagonist pair for Tie-2 receptor signaling although this dichotomous action appears to be context, dose, and duration specific (Maisonpierre et al., Science 277: 55-60 (1997), Teichert-Kuliszewska et al., Cardiovasc. Res. 49: 659-670 (2001), Saharinen et al., J. Cell Biol. 169: 239-243 (2005)).

We have discovered that Ang-2 is both a marker of and a mediator of pathologic vascular leak in the lung. We have shown that Ang-2 levels are elevated in patients with sepsis and impairment in gas exchange and with vascular leak disease associated with high dose I1-2 therapy. We have also shown that Ang-2 distorts endothelial cell architecture and contributes to vascular leak and pulmonary injury, at least in part, through binding to Tie-2 and activation of myosin light chain phosphorylation via Rho-kinase. We have also discovered that sepsis is a condition in which an imbalance in the Ang-1/Ang-2/Tie-2 pathway leads to increased microvascular permeability and ultimately to vascular leak. Our results show that Ang-1 regulates the endothelial cytoskeleton and protects against vascular leak through activation of p190RhoGAP and inhibition of RhoA. Ang-2 levels can be used as a measurement tool to predict, diagnose, or stratify patients who have or are at risk for developing a disorder characterized by vascular leak, hypotension or a procoagulant state. Antagonists to Ang-2, including antagonists to any signaling proteins activated downstream of Ang-2, are useful as therapeutics for the treatment or prevention of vascular leak syndromes, hypotension, or a procoagulant state.

Accordingly, the invention features a method of treating or preventing a vascular leak in a subject that includes the step of administering to the subject an Ang-2 antagonist for a time and in an amount sufficient to treat or prevent the vascular leak disorder. The method can be used to treat vascular leak associated with a vascular leak disorder, such as sepsis; pneumonia; ALI; ARDS; vascular leak associated with IL-2 therapy or rituximab therapy; idiopathic capillary leak syndrome; pre-eclampsia; eclampsia; hypotensive states due to sepsis; heart failure; trauma; infection; pulmonary aspiration of stomach contents; pulmonary aspiration of water; near drowning; burns; inhalation of noxious fumes; fat embolism; blood transfusion; amniotic fluid embolism; air embolism; edema; organ failure; poisoning; radiation; acute and chronic vascular rejection; pancreatitis; trauma; vasculitis; C1 esterase inhibitor deficiency; TNF receptor associated periodic fever syndrome; massive blood transfusion; anaphylaxis; post-lung or post-heart-lung transplant; and ovarian hyperstimulation syndrome. In one embodiment, the method is used to treat a vascular leak associated with high dose IL-2 therapy.

Ang-2 antagonists include any compound that reduces or inhibits the expression levels or biological activity of Ang-2 or signaling proteins downstream of Ang-2. Non-limiting examples of an Ang-2 antagonist compound include an antibody that specifically binds Ang-2; isolated Ang-1 polypeptide, or biologically active fragments thereof; Ang-2 binding proteins that block Ang-2 binding to Tie-2 receptor; Tie-2 binding proteins that specifically block Ang-2 binding to Tie-2; soluble Tie-2 fragments that specifically bind to Ang-2; dominant active mutants of Tie-2; antibodies that specifically bind to Tie-2 and selectively inhibit Ang-2 binding to Tie-2; compounds that inhibit MLC phosphorylation; compounds that inhibit RhoA GTPase activity or expression levels (e.g., small RNA, antisense nucleobase oligomer, or an antibody); compounds that inhibit Rho kinase activity or expression levels (e.g., small RNA, antisense nucleobase oligomer, or an antibody); compounds that activate Rac1 activity or increase Rac1 expression levels, and compounds that activate p190RhoGAP or increase p190RhoGAP expression levels. One preferred compound is a purified antibody, or fragment thereof, that specifically binds Ang-2, such as L1-7(N), 2xCon4, L-10(N), or AB536. Desirably, the antibody is L1-7(N).

Additional Ang-2 antagonists that decrease the expression level of Ang-2 and are useful in the therapeutic methods of the invention include an antisense nucleobase oligomer that is at least 95% complementary to at least a portion of an Ang-2 nucleic acid sequence. Desirably, the antisense nucleobase oligomer is 8 to 30 nucleotides in length. The antisense nucleobase oligomer can also contain at least 40, 60, 85, 120, or more consecutive nucleotides that are complementary to Ang-2 mRNA or DNA, and may be as long as the full-length mRNA or gene. In yet another preferred embodiment, the Ang-2 antagonist compound is a small RNA having at least one strand that is at least 80%, preferably 85%, 90%, 95%, 99%, or 100% complementary to at least a portion of an Ang-2 nucleic acid sequence, or a complementary sequence thereof. The small RNA can be either single-stranded or double-stranded and is at least 15 nucleotides, preferably, 17, 18, 19, 20, 21, 22, 23, 24, 25, 26, 27, 28, 29, 30, 31, 32, 33, 34, or 35, nucleotides in length and even up to 50 or 100 nucleotides in length (inclusive of all integers in between). Preferably, the small RNA is double stranded and is 19 to 25 nucleotides in length and is capable of mediating RNAi. Small RNA includes siRNA, microRNA, or shRNA molecules.

In one embodiment, the invention features a method of treating a vascular leak associated with high dose (HD) IL-2 therapy that includes administering an antibody that specifically binds to Ang-2. Preferred antibodies include L1-7(N), 2xCon4, L-10(N), or fragments or derivatives (e.g., chimeric, humanized, fully human) thereof.

In preferred embodiments of any of the therapeutic methods described above, the Ang-2 antagonist compounds can be used alone or in combination with additional Ang-2 antagonist compounds. One exemplary combination includes an Ang-2 inhibitor such as an antagonistic antibody in combination with an isolated Ang-1 polypeptide, or biologically active fragments thereof, or a compound that activates p190RhoGAP or Rac1. Any of the Ang-2 antagonist compounds described above can also be used in combination with any compound known in the art for the treatment of vascular leak. Examples include an antibiotic, drotrecogin alpha, a corticosteroid, vasopressin, and/or the administration of a mechanical ventilation device.

In one embodiment of the therapeutic methods of the invention, the Ang-2 antagonist compound is administered to the subject within 7 days, 6 days, 5 days, 4 days, 3 days, 2 days, 1 day, 18 hours, 12 hours, 6 hours or less after identification of the vascular leak or the vascular leak disorder in the subject. In additional embodiments, the Ang-2 antagonist is administered intravenously or via bronchoscopic injection.

In another aspect, the invention features a method of diagnosing a subject as having, or at risk of having, a vascular leak, that includes measuring the level of an Ang-2 polypeptide in a sample from the subject. In one embodiment, the level of Ang-2 measured can be compared to an absolute level of Ang-2 that is known to be a normal level or compared to a known normal reference sample. In one embodiment, a level of Ang-2 greater than 5 ng/ml, 10 ng/ml or 20 ng/ml is a diagnostic indicator of a vascular leak or a risk of having a vascular leak.

In another embodiment, the method includes comparing the Ang-2 polypeptide level to the Ang-2 polypeptide level in a normal reference, wherein an increase (e.g., at least 10%, 20%, 30%, 40%, 50%, 60%, 70%, 80%, 90%, 95% or more) in the level of the Ang-2 polypeptide relative to the normal reference is a diagnostic indicator of vascular leak or a risk of developing a vascular leak. In one example, the normal reference is a prior sample or level taken from the subject. In another example the normal reference is a sample or level from a subject that does not have a vascular leak disorder. In another example, the method includes comparing the Ang-2 polypeptide level to a the Ang-2 polypeptide level in a positive reference wherein a level about equal to (e.g., within 20%, 15%, 10%, 5%, 4%, 3%, 2%, 1%, or less than 1%) or greater than the level in a positive reference is a diagnostic indicator of vascular leak or a risk of developing a vascular leak. In another example the measuring of levels is done on two or more occasions and an alteration in the levels between measurements is a diagnostic indicator of a vascular leak or a risk of developing a vascular leak. It will be understood by the skilled artisan that for diagnostic methods that include the comparing of the Ang-2 level to a reference level, particularly a prior sample taken from the same subject, a change in time with respect to the baseline level can be used as a diagnostic indicator of vascular leak or a risk of developing a vascular leak. In this embodiment, if a subject has a baseline level of 1 ng/ml Ang-2 and this level increases over time to a level of 4 ng/ml Ang-2, this is considered a diagnostic indicator of vascular leak or a risk of developing a vascular leak based on the percent change over baseline.

Any of the diagnostic methods of the invention can further include measuring the level of at least one cytokine selected from the group consisting of TNF-α, IL-1, IL-6, VEGF, or PlGF in a sample from said subject. Generally, an increase in the level of TNF-α, VEGF, or PlGF as compared to a normal reference sample or level is a diagnostic indicator of a vascular leak or a risk of developing a vascular leak.

In preferred embodiments of any of the above diagnostic methods of the invention, the measuring of the Ang-2 polypeptide is done using an immunological assay, such as an ELISA.

In another aspect, the invention features a method of diagnosing a subject as having, or having a predisposition to, a vascular leak, that includes measuring the level of an Ang-2 nucleic acid molecule in a sample from the subject and comparing it to a reference, wherein an alteration (e.g., an increase of at least 10%, 20%, 30%, 40%, 50%, 60%, 70%, 80%, 90%, 95% or more) in the levels compared to a reference is a diagnostic indicator of a vascular leak or a risk of developing a vascular leak.

For any of the diagnostic or monitoring methods described herein, the method is used to diagnose or monitor a vascular leak in a subject having sepsis; pneumonia; ALI; ARDS; IL-2 or rituximab therapy; idiopathic capillary leak syndrome; pre-eclampsia; eclampsia; hypotensive states due to sepsis; heart failure; trauma; infection; pulmonary aspiration of stomach contents; pulmonary aspiration of water; near drowning; burns; inhalation of noxious fumes; fat embolism; blood transfusion; amniotic fluid embolism; air embolism; edema; organ failure; poisoning; radiation; acute and chronic vascular rejection; pancreatitis; trauma; vasculitis; C1 esterase inhibitor deficiency; TNF receptor associated periodic fever syndrome; massive blood transfusion; anaphylaxis; post-lung or post-heart-lung transplant; or ovarian hyperstimulation syndrome. Desirably, the method is used to diagnose a vascular leak or a risk of developing a vascular leak in a subject undergoing high dose IL-2 therapy.

In one embodiment, the levels are measured on two or more occasions and a change in the levels between measurements is a diagnostic indicator of a vascular leak or a risk of developing a vascular leak. For example, the Ang-2 nucleic acid levels can be compared to a normal reference, wherein an increase (e.g., at least 105, 20%, 30%, 40%, 50%, 60%, 70%, 80%, 90%, 95%, or more) in the level of the Ang-2 nucleic acid is a diagnostic indicator of a vascular leak or a risk of developing a vascular leak. In one example, the normal reference is a prior sample or level taken from the subject. In another example the normal reference is a sample or level from a subject that does not have a vascular leak or a vascular leak disorder. In another example, the method includes comparing the Ang-2 nucleic acid level to the Ang-2 nucleic acid level in a positive reference wherein a level about equal to (e.g., within 20%, 15%, 10%, 5%, 4%, 3%, 2%, 1%, or less than 1%) or greater than the level in a positive reference is a diagnostic indicator of vascular leak or a risk of developing a vascular leak.

In yet another aspect, the invention features a method of diagnosing a subject as having, or a risk of developing a vascular leak, that includes determining the nucleic acid sequence of an Ang-2 gene in a sample from a subject and comparing it to a reference sequence, wherein an alteration in the subject's Ang-2 nucleic acid sequence that is an alteration that changes the expression level of the gene product in the subject diagnoses the subject with a vascular leak disorder or a risk of developing a vascular leak disorder.

In preferred embodiments of any of the diagnostic aspects of the invention, the sample is a bodily fluid (e.g., urine, blood, serum, plasma, and cerebrospinal fluid), cell, or tissue sample from the subject in which the Ang-2 is normally detectable. In additional preferred embodiments, the subject is a mammal (e.g., human, bovine, equine, canine, ovine, or feline), preferably a human.

Any of the diagnostic methods described herein can be used to monitor and manage vascular leak in a subject. In one example, the Ang-2 levels are monitored in a subject undergoing therapy for vascular leak or a vascular leak disorder. The levels can be measured and compared to a positive reference sample or measured on two or more occasions and a change over time is determined. If the Ang-2 levels decrease over time, this is considered an indicator of an improvement in the vascular leak or vascular leak disorder. In another example, if the subject has an Ang-2 level less than 10 ng/ml serum, preferably less than 5 ng/ml serum, this is also an indicator of an improvement in the vascular leak or vascular leak disorder. The diagnostic methods can also be used to determine the therapeutic dosage of the Ang-2 antagonist compound. In preferred embodiments, the method is used to monitor a subject undergoing HD IL2 therapy and is being treated for vascular leak or a risk of developing a vascular leak.

Any of the monitoring methods of the invention can further include measuring the level of at least one cytokine selected from the group consisting of TNF-α, IL-1, IL-6, VEGF, or PlGF in a sample from said subject. Generally, an increase in the level of TNF-α, VEGF, or PlGF as compared to a normal reference sample or level is a diagnostic indicator of a vascular leak or a risk of developing a vascular leak.

In preferred embodiments of any of the above monitoring methods of the invention, the measuring of the Ang-2 polypeptide is done using an immunological assay, such as an ELISA.

In another aspect, the invention features a kit for the diagnosis of a vascular leak, or a risk of developing a vascular leak, in a subject that includes a nucleic acid molecule having an Ang-2 nucleic acid sequence or a sequence complementary thereto, or any combination thereof, and instructions for using the nucleic acid molecule to diagnose a vascular leak or a risk of developing a vascular leak or to monitor or manage vascular leak.

In another aspect the invention features a kit for the diagnosis of a vascular leak, or a risk of developing a vascular leak, in a subject comprising an Ang-2 binding molecule and instructions for the use of the Ang-2 binding molecule for the diagnosis of the vascular leak, or a risk of developing a vascular leak, or to monitor and manage vascular leak. Desirably, the Ang-2 binding molecule is an antibody, or antigen-binding fragment thereof, that specifically binds Ang-2 polypeptide. In additional embodiments, the kit further comprises a polypeptide that specifically binds at least one cytokine selected from the group consisting of TNF, IL-1, IL-6, VEGF, and PlGF.

In another aspect, the invention provides a composition comprising a purified antibody or antigen-binding fragment thereof that specifically binds Ang-2. In one preferred embodiment, the antibody reduces or inhibits the biological activity of Ang-2. In another embodiment, the antibody is a monoclonal antibody. In other preferred embodiments, the antibody or antigen-binding fragment thereof is a human or humanized antibody. In other embodiments, the antibody lacks an Fc portion, is an F(ab′)₂, an Fab, or an Fv structure. In other embodiments, the antibody or antigen-binding fragment thereof is present in a pharmaceutically acceptable carrier.

In another aspect, the invention features a method of inducing a vascular leak in a subject in need thereof, that includes administering to the subject an Ang-2 agonist compound for a time and in an amount sufficient to induce a vascular leak in the subject. In one embodiment, the subject has a brain tumor.

The Ang-2 agonist compound can be any compound that shifts the GTPase balance in favor of RhoA activity over Rac1. Examples of an Ang-2 agonist compound include a purified Ang-2 protein, an isolated nucleic acid molecule encoding an Ang-2 polypeptide; an agonistic anti-Ang-2 antibody; a compound that binds to Tie-2 and blocks Ang-1 binding but not Ang-2 binding; a compound that induces MLC phosphorylation; a compound that activates Rho kinase activity; a compound that inhibits Rac1 or p190RhoGAP biological activity or expression (e.g., small RNA, antisense nucleobase oligomer, or an antibody); a compound that inhibits Ang-1 biological activity or expression (e.g., small RNA, antisense nucleobase oligomer, or an antibody); and a compound that induces RhoA biological activity or expression levels (e.g., a purified RhoA protein).

By “acute lung injury” or “ALI” is meant a lung disorder whose manifestations include all of the following: bilateral infiltrates on chest x-ray; PaO2/FiO2<300; ruling out acute left ventricular heart failure as the sole etiology of the above two; acute left ventricular heart failure can coexist with ALI. ALI can also be characterized as hypoxemic respiratory failure, as defined by Bernard et al. Am. J. Respir. Crit. Care Med. 149:818-824 (1994). ALI is frequently encountered as a clinical prelude to acute respiratory distress syndrome.

A severe form of ALI is referred to as “acute respiratory distress syndrome” or “ARDS.” ARDS is a lung disorder arising from multiple etiologies whose manifestations include all of the following: bilateral infiltrates on chest x-ray; severe impairment in oxygenation of blood, as indicated by a ratio of oxygen content in blood (PaO2) to inspired fraction of air containing oxygen (FiO2) of less than or equal to 200 (PaO2/FiO2<200); and ruling out acute left ventricular heart failure as the sole cause of the above two. Acute left ventricular heart failure can coexist with ARDS.

By “alteration” is meant a change (increase or decrease). The alteration can be in the expression levels of a nucleic acid or polypeptide (e.g., Ang-2) as detected by standard art known methods such as those described below. As used herein, an alteration includes a 10% change in expression levels, preferably a 25% change, more preferably a 40%, 50%, 60%, 70%, 80%, 90%, 95%, 96%, 97%, 98%, 99% or greater change in expression levels. “Alteration” can also include a change (increase or decrease) in the biological activity of a polypeptide of the invention (e.g., Ang-2). As used herein, an alteration includes a 10% change in biological activity, preferably a 25% change, more preferably a 40%, 50%, 60%, 70%, 80%, 90%, 95%, 96%, 97%, 98%, 99% or greater change in biological activity. Examples of biological activity for Ang-2 polypeptides are described below.

By “angiopoietin-1” or “Ang-1” is meant a polypeptide, or a nucleic acid sequence that encodes it, that is substantially identical or homologous to any of the following amino acid sequences: SEQ ID NO: 3 (polypeptide), SEQ ID NO: 4 (nucleic acid), GenBank Accession Numbers NM_{—}001146, NP_{—}001137, and BAB91325, or fragments thereof, or that has Ang-1 biological activity, as described below, or preferably both. Ang-1 is a secreted protein that is approximately 55 kDa in size and the glycosylated forms can be approximately 70 kDa. Ang-1 nucleic acid molecules encode an Ang-1 polypeptide and preferably have substantial identity to the nucleic acid sequence set forth in SEQ ID NO: 4. Ang-1 can also include fragments, derivatives, or analogs of Ang-1 that preferably retain at least 25%, 30%, 40%, 50%, 60%, 70%, 80%, 90%, 95%, 96%, 97%, 98%, 99%, or more Ang-1 biological activity (e.g., binding to the Tie-2 receptor). Ang-2 polypeptides may be isolated from a variety of sources, such as from mammalian tissue or cells or from another source, or prepared by recombinant or synthetic methods. The term “Ang-1” also encompasses modifications to the polypeptide, fragments, derivatives, analogs, and variants of the Ang-1 polypeptide. Preferred Ang-1 fragments or variants useful in the methods of the invention include fragments or variants that can antagonize the function of Ang-2, for example, by binding to the Tie-2 receptor and blocking Ang-2 binding to the receptor, or can activate Rac1 or p190RhoGAP which can inhibit or suppress RhoA and Rho kinase activity. Ang-1 is also known as “ANGPT1,” “AGPT,” “AGP1,” and “Angiopoietin-1 precursor” all of which are encompassed by the term Ang-1.

By “Ang-1 biological activity” is meant any of the following activities: binding to the Tie-2 receptor, activation of the Tie-2 receptor, induction of Tie-2 phosphorylation, pro-angiogenic or anti-angiogenic activity depending on the environment (Stoeltzing et al., Cancer Res. 63:3370-3377 (2003)), activation of p190RhoGAP, activation of Rac1, downregulation or inhibition of RhoA GTPase or Rho kinase activity, inhibition of vascular permeability, promotion of tumor angiogenesis and tumor vessel plasticity, promotion of endothelial cell survival, anti-inflammatory activity, reduction in expression of inflammatory molecules (e.g., ICAM1) and blood vessel development. Assays for Ang-1 biological activity are known in the art or described herein and include Tie-2 receptor binding assays, Tie-2 receptor activation assays, Tie-2 phosphorylation assays, in vitro and in vivo angiogenesis assays, and vascular permeability assays.

By “angiopoietin-2” or “Ang-2” is meant a polypeptide, or a nucleic acid sequence that encodes it, that is substantially identical or homologous to any of the following amino acid sequences: SEQ ID NO: 1 (polypeptide), SEQ ID NO: 2 (nucleic acid), GenBank Accession Numbers NM_{—}001147, NP_{—}001138, and BAA95590 or that has Ang-2 biological activity, as described below, or preferably both. Ang-2 is a secreted protein that is approximately 55 kDa in size and the glycosylated forms can be approximately 70 kDa. (See, for example, Maisonpierre et al. Science 277:55 (1997)). Ang-2 nucleic acid molecules encode an Ang-2 polypeptide and preferably have substantial identity to the nucleic acid sequence described in SEQ ID NO: 2. Ang-2 can also include fragments, derivatives, or analogs of Ang-1 and that preferably retain at least 25%, 30%, 40%, 50%, 60%, 70%, 80%, 90%, 95%, 96%, 97%, 98%, 99%, or more Ang-2 biological activity. Ang-2 polypeptides may be isolated from a variety of sources, such as from mammalian tissue or cells or from another source, or prepared by recombinant or synthetic methods. The term “Ang-2” also encompasses modifications to the polypeptide, fragments, derivatives, analogs, and variants of the Ang-2 polypeptide. Ang-2 is also known as “ANGPT2” and “Angiopoietin-2 precursor” both of which are encompassed by the term Ang-2.

By “Ang-2 biological activity” is meant any of the following activities: antagonism of Ang-1 activity, binding to the Tie-2 receptor, inhibition of phosphorylation of the Tie-2 receptor, inhibition of Tie-2 receptor signaling, disruption of blood vessel formation, destabilization of blood vessels, induction of vascular permeability, induction in the expression of inflammatory molecules such as ICAM-1, and modulation of angiogenesis. Assays for Ang-2 activity are known in the art or described herein and include Tie-2 receptor binding assays, Tie-2 receptor activation assays, Tie-2 phosphorylation assays, in vitro and in vivo angiogenesis assays, and vascular permeability assays.

By “Ang-2 antagonist” is meant any compound (e.g., small molecule, polypeptide, nucleic acid molecule, antibody, or fragments or functional derivatives thereof) that inhibits, reduces, or prevents Ang-2 expression or biological activity, for example by reducing or inhibiting Ang-2 protein synthesis, reducing Ang-2 nucleic acid levels, preventing or inhibiting Ang-2 binding to Tie-2 receptor, or reducing or inhibiting the Ang-2 signaling pathway downstream of the Tie-2 receptor. Non-limiting examples of Ang-2 antagonists include antibodies (e.g., neutralizing antibodies), or fragments thereof, that specifically bind to Ang-2; Ang-1, or biologically active peptide fragments thereof; nucleic acid molecules that decrease Ang-2 expression (e.g., small RNA, antisense); Ang-2 binding proteins that prevent binding to Tie-2 receptor; antibodies that specifically bind to Tie-2 and prevent Ang-2 binding but not Ang-1 binding; soluble Tie-2 fragments that can bind to Ang-2; dominant active Tie-2 mutants that are constitutively active (Vikkula et al., Cell 87: 1181-1190 (1996)); antibodies that specifically bind to Tie-2 and selectively inhibit Ang-2 binding to Tie-2. Examples of antibodies that specifically bind Ang-2 include L1-7(N) (Oliner et al., Cancer Cell 6:507-516 (2004)), anti-Ang-2 antibodies from Research Diagnostics Inc., (e.g., catalog nos. RDI-ANGIOP2XabR, RDI-ANG218NabG, and RDI-MANGIOP2abrx) and from AbCam Inc. (e.g., catalog nos. Ab18518, Ab8452, and Ab10601). Non-limiting examples of Ang-2 antagonists that function downstream of the Tie-2 receptor include activators of p190RhoGAP or Rac1 activity or expression levels, inhibitors of MLC phosphorylation, inhibitors of RhoA GTPAse activity or expression levels; inhibitors of Rho kinase activity or expression levels, and inducers of Tie-2 phosphorylation. Desirably, the Ang-2 antagonist will inhibit, reduces, or prevents Ang-2 expression or biological activity by at least 10%, preferably 20%, 30%, 40%, 60%, 80%, 90%, 95%, or more. Ang-2 compounds can be assayed for efficacy using any of the structural, function, and molecular assays described herein or known in the art. Examples of such assays include functional assays (e.g., phenotypic observations of spindle phenotype, thick actin stress fibers, and paracellular gap formation; determination of an increase in vascular barrier integrity; and determination of a decrease in the leak itself), structural assays (e.g., FITC-albumin permeability assay, transendothelial resistance (TER) measurements), and molecular assays (e.g., inhibition of MLC phosphorylation, inhibition of Rho kinase activity, induction of Tie-2 phosphorylation as molecular assays, activation of PI-3 kinase activity, activation of Rac1, activation of p190RhoGAP, and activation of protein kinase C activity). In one example, the Ang-2 antagonist compound, or functional derivative thereof, will increase vascular barrier integrity (e.g. as assessed by TER) by at least about 10%, 20%, 30%, 40%, 50%, 60%, 70%, 80%, 90%, or 100% in a TER assay relative to a control (i.e. the same assay where the cells have not been exposed to Ang-2 antagonist or functional derivative thereof).

By “antisense nucleobase oligomer” is meant a nucleobase oligomer, regardless of length, that is complementary to at least a portion of the coding strand or mRNA of an Ang-2 gene. By a “nucleobase oligomer” is meant a compound that includes a chain of at least eight nucleobases, preferably at least twelve, and most preferably at least sixteen bases, joined together by linkage groups. Included in this definition are natural and non-natural oligonucleotides, both modified and unmodified, as well as oligonucleotide mimetics such as Protein Nucleic Acids, locked nucleic acids, and arabinonucleic acids. Numerous nucleobases and linkage groups may be employed in the nucleobase oligomers of the invention, including those described in U.S. Patent Publication Nos. 20030114412 (see for example paragraphs 27-45 of the publication) and 20030114407 (see for example paragraphs 35-52 of the publication), incorporated herein by reference. The nucleobase oligomer can also be targeted to the translational start and stop sites. Preferably the antisense nucleobase oligomer comprises from about 8 to 30 nucleotides. The antisense nucleobase oligomer can also contain at least 40, 60, 85, 120, or more consecutive nucleotides that are complementary to Ang-2 mRNA or DNA, and may be as long as the full-length mRNA or gene.

By “compound” is meant any small molecule chemical compound, antibody, nucleic acid molecule, or polypeptide, or fragments thereof.

By “expression” is meant the detection of a gene or polypeptide by standard art known methods. For example, polypeptide expression is often detected by Western blotting, DNA expression is often detected by Southern blotting or polymerase chain reaction (PCR), and RNA expression is often detected by Northern blotting, PCR, or RNAse protection assays.

By “fragment” is meant a portion of a polypeptide or nucleic acid molecule that contains, preferably, at least 10%, 20%, 30%, 40%, 50%, 60%, 70%, 80%, 90%, 95% or more of the entire length of the reference nucleic acid molecule or polypeptide. A fragment may contain 10, 20, 30, 40, 50, 60, 70, 80, 90, or 100, 200, 300, 400, 500, 600, 700, 800, 900, 1000, 1100, 1200, 1300, 1400, 1500 or more nucleotides, up to the full length of the nucleic acid, or 10, 20, 30, 40, 50, 60, 70, 80, 90, 100, 150, 200, 250, 300, 350, 400, 450, or 500 amino acids or more up to the full length of the protein. Preferred fragments useful in the therapeutic methods of the invention for the treatment of vascular disorders include Ang-1 peptide fragments that retain Ang-1 biological activity and soluble Tie-2 fragments that can bind to Ang-2. Fragments can be modified as described herein and known in the art.

By “heterologous” is meant any two or more nucleic acid or polypeptide sequences that are not normally found in the same relationship to each other in nature. For instance, the nucleic acid is typically recombinantly produced, having two or more sequences, e.g., from unrelated genes arranged to make a new functional nucleic acid, e.g., a promoter from one source and a coding region from another source. Similarly, a heterologous polypeptide will often refer to two or more subsequences that are not found in the same relationship to each other in nature (e.g., a fusion protein).

By “homologous” is meant any gene or polypeptide sequence that bears at least 30% homology, more preferably 40%, 50%, 60%, 70%, 80%, and most preferably 90%, 95%, 96%, 97%, 98%, 99%, or more homology to a known gene or polypeptide sequence over the length of the comparison sequence. A “homologous” polypeptide can also have at least one biological activity of the comparison polypeptide. For polypeptides, the length of comparison sequences will generally be at least 16 amino acids, preferably at least 20 amino acids, more preferably at least 30, 40, 50, 60, 70, 80, 90, 100, 150, 200, 250, 300, 350, 400, 450, or 500 amino acids or more. For nucleic acids, the length of comparison sequences will generally be at least 50 nucleotides, preferably at least 60, 70, 80, 90, 100, 150, 200, 250, 300, 350, 400, 450, 500, 550, 600, 700, 800, 900, 100, 1100, 1200, 1300, 1400, 1500, or more. “Homology” can also refer to a substantial similarity between an epitope used to generate antibodies and the protein or fragment thereof to which the antibodies are directed. In this case, homology refers to a similarity sufficient to elicit the production of antibodies that can specifically recognize the protein or polypeptide.

By “humanized antibody” is meant an immunoglobulin amino acid sequence variant or fragment thereof that is capable of binding to a predetermined antigen. Ordinarily, the antibody will contain both the light chain as well as at least the variable domain of a heavy chain. The antibody also may include the CH1, hinge, CH2, CH3, or CH4 regions of the heavy chain. The humanized antibody comprises a framework region (FR) having substantially the amino acid sequence of a human immunoglobulin and a complementarity determining region (CDR) having substantially the amino acid sequence of a non-human immunoglobulin (the “import” sequences).

Generally, a humanized antibody has one or more amino acid residues introduced into it from a source that is non-human. In general, the humanized antibody will comprise substantially all of at least one, and typically two, variable domains (Fab, Fab′, F(ab′)₂, Fabc, Fv) in which all or substantially all of the CDR regions correspond to those of a non-human immunoglobulin and all or substantially all of the FR regions are those of a human immunoglobulin consensus sequence. The humanized antibody optimally will comprise at least a portion of an immunoglobulin constant region (Fc), typically that of a human immunoglobulin. By “complementarity determining region (CDR)” is meant the three hypervariable sequences in the variable regions within each of the immunoglobulin light and heavy chains. By “framework region (FR)” is meant the sequences of amino acids located on either side of the three hypervariable sequences (CDR) of the immunoglobulin light and heavy chains.

The FR and CDR regions of the humanized antibody need not correspond precisely to the parental sequences, e.g., the import CDR or the consensus FR may be mutagenized by substitution, insertion or deletion of at least one residue so that the CDR or FR residue at that site does not correspond to either the consensus or the import antibody. Such mutations, however, will not be extensive. Usually, at least 75%, preferably 90%, and most preferably at least 95% of the humanized antibody residues will correspond to those of the parental FR and CDR sequences.

By “p190RhoGAP” is meant a multi-domain 190 kDa protein that localizes to the cytoplasm of cultured cells and appears to function as an inhibitor of cell proliferation and inducer of apoptosis. p190RhoGAP contains a RhoGAP domain that activates the intrinsic GTPase activity of the Rho family of small GTPases, which regulate actin cytoskeleton rearrangements in response to growth factor or integrin stimulation. p190RhoGAP is also tyrosine phosphorylated and a substrate of c-Src.

By “pharmaceutically acceptable carrier” is meant a carrier that is physiologically acceptable to the treated mammal while retaining the therapeutic properties of the compound with which it is administered. One exemplary pharmaceutically acceptable carrier substance is physiological saline. Other physiologically acceptable carriers and their formulations are known to one skilled in the art and described, for example, in Remington's Pharmaceutical Sciences, (20^{th} edition), ed. A. Gennaro, 2000, Lippincott, Williams & Wilkins, Philadelphia, Pa.

By “preventing” is meant prophylactic treatment of a subject who is not yet ill, but who is susceptible to, or otherwise at risk of, developing a particular disease. Preferably, a subject is determined to be at risk of developing any type of vascular leak, for example resulting from sepsis or interleukin-2 therapy, using the diagnostic methods known in the art or described herein. For example, in the case of a patient already diagnosed with sepsis, “preventing” can refer to the prevention of severe sepsis, lung failure, or death. In another example, in the case of a patient undergoing IL-2 therapy, prevention can refer to prevention of the onset of vascular leak.

A “promoter” is defined as an array of nucleic acid control sequences that direct transcription of a nucleic acid. As used herein, a promoter includes necessary nucleic acid sequences near the start site of transcription, such as, in the case of a polymerase II type promoter, a TATA element. A promoter also optionally includes distal enhancer or repressor elements, which can be located as much as several thousand base pairs from the start site of transcription. A “constitutive” promoter is a promoter that is active under most environmental and developmental conditions. An “inducible” promoter is a promoter that is active under environmental or developmental regulation. The term “operably linked” refers to a functional linkage between a nucleic acid expression control sequence (such as a promoter, or array of transcription factor binding sites) and a second nucleic acid sequence, wherein the expression control sequence directs transcription of the nucleic acid corresponding to the second sequence.

By “protein,” “polypeptide,” or “polypeptide fragment” is meant any chain of more than two amino acids, regardless of post-translational modification (e.g., glycosylation or phosphorylation), constituting all or part of a naturally occurring polypeptide or peptide, or constituting a non-naturally occurring polypeptide or peptide. A polypeptide (or fragment thereof) may be said to be “isolated” or “substantially pure” when physical, mechanical or chemical methods have been employed to remove the polypeptide from cellular constituents. An “isolated polypeptide,” “substantially pure polypeptide,” or “substantially pure and isolated polypeptide” is typically considered removed from cellular constituents and substantially pure when it is at least 60% by weight, free from the proteins and naturally occurring organic molecules with which it is naturally associated. Preferably, the polypeptide is at least 75%, more preferably at least 90%, and most preferably at least 99% by weight pure. A substantially pure polypeptide may be obtained by standard techniques, for example, by extraction from a natural source (e.g., lung tissue or cell lines), by expression of a recombinant nucleic acid encoding the polypeptide, or by chemically synthesizing the polypeptide. Purity can be measured by any appropriate method, e.g., by column chromatography, polyacrylamide gel electrophoresis, or HPLC analysis. Alternatively, a polypeptide is considered isolated if it has been altered by human intervention, or placed in a location that is not its natural site, or if it is introduced into one or more cells.

By “purified” or “isolated” is meant is at least 60%, by weight, free from proteins and other molecules (e.g., naturally occurring or synthetic) with which it is naturally associated. Preferably, the preparation is at least 75%, more preferably 90%, and most preferably at least 99%, by weight.

By “reduce or inhibit” is meant the ability to cause an overall decrease preferably of 20% or greater, more preferably of 50% or greater, and most preferably of 75%, 80%, 85%, 90%, 95%, or greater. For therapeutic applications, reduce or inhibit can refer to the symptoms of the disorder being treated or the presence or extent of vascular leak. Symptoms of the disorder include impairment in the inability to oxygenate the blood as well as impairment in the ability to ventilate the lungs (as with mechanical ventilation). These impairments generally mandate intensification of ventilation strategies (e.g. introduction of greater positive pressure to inflate stiff lungs). Other clinical endpoints that may be reduced or inhibited include the following: overall survival, days of ICU care required, long-term oxygen requirement, requirement for pulmonary bypass procedures such as ECMO (extracorporeal membrane oxygenation), development of pneumothorax, requirement for immunomodulatory therapies, such as glucocorticoids, development of chronic patterns of injury as a result of severe ARDS such as bronchiolitis obliterans and pulmonary fibrosis. For diagnostic or monitoring applications, reduce or inhibit can refer to a decrease in the level of protein or nucleic acid, detected by the aforementioned assays (see “expression”).

By “reference” is meant any sample, standard, or level that is used for comparison purposes. A “normal reference sample” can be a prior sample taken from the same subject prior to the onset of vascular leak or hypotension (e.g., resulting from sepsis or IL-2 therapy) or during the early stages of vascular leak, hypotension, or sepsis; a sample from a subject not having vascular leak or hypotension; a subject that has been successfully treated for vascular leak or hypotension (e.g., resulting from sepsis or IL-2 therapy); or a sample of a purified reference Ang-2 polypeptide at a known normal concentration. By “reference standard or level” is meant a value or number derived from a reference sample. A normal reference standard or level can be a value or number derived from a normal subject that is matched to the sample subject by at least one of the following criteria: age, weight, disease stage, and overall health. In one example, a normal reference level of Ang-2 is less than 5 ng/ml serum, preferably less than 4 ng/ml, 3 ng/ml, 2 ng/ml, or less than 1 ng/ml serum. A “positive reference” sample, standard or value is a sample or value or number derived from a subject that is known to have a vascular leak or hypotension (e.g., resulting from sepsis, IL-2 therapy, or any of the order disorders described herein) that is matched to the sample subject by at least one of the following criteria: age, weight, disease stage, and overall health. For example, a positive reference value for Ang-2 is greater than 5 ng/ml serum, preferably greater than 10 ng/ml serum or most preferably greater than 20 ng/ml serum.

By “Rho” is meant a member of the Rho family of GTPases. The Rho family of GTPases is a family of proteins that couples extracellular signaling events to changes in cellular function including endocytosis. The Rho family is comprised of at least fifteen members and their isoforms including: Rho subfamily (A, B, C isoforms), Rac subfamily (1, 2, 3 isoforms), Cdc42 (Cdc42Hs and G25K splice variants), Chp, Rnd subfamily (Rnd1, Rnd2, Rnd3 isoforms), RhoD, RhoG, RhoH, and TC10. (See Wherlock et al., J. Cell Sci. 115:239-240 (2002)). For each subfamily, it will be understood that while the specification refers specifically to one family member (e.g., RhoA or Rac1), it will be understood by the skilled artisan that additional members of the subfamily may be used in the invention as well. Rho family members, like all GTPases, cycle between an inactive GDP-bound state and an active GTP-bound state. The activity of Rho GTPases is modulated by several accessory proteins including guanine nucleotide exchange factors (GEFs), GTPase-activating proteins (GAPs), and GDP dissociation inhibitors (GDIs). GEFs, as their name implies, stimulate Rho family members to exchange GDP for GTP; GTPase activation is the result. GAPs (e.g., p190RhoGAP) stimulate the Rho GTPase to hydrolyze its bound GTP, returning the Rho protein to its inactive GDP-bound state. GDIs preferentially bind Rho-GDP and modulate the activation and targeting of Rho-GDP to the membrane. Upon activation, Rho GTPases interact with a plethora of downstream effector molecules that, in turn, modulate cellular function.

By “Rho kinase” is meant a serine threonine kinase that serves as a substrate for Rho family members and mediates cellular functions including focal adhesions, motility, smooth muscle contraction, and cytokinesis. Rho kinase also modulates the phosphorylation of myosin light chain (MLC) of myosin.

By “small RNA” is meant any RNA molecule, either single-stranded or double-stranded” that is at least 15 nucleotides, preferably, 17, 18, 19, 20, 21, 22, 23, 24, 25, 26, 27, 28, 29, 30, 31, 32, 33, 34, or 35, nucleotides in length and even up to 50 or 100 nucleotides in length (inclusive of all integers in between). Preferably, the small RNA is capable of mediating RNAi. As used herein the phrase “mediates RNAi” refers to the ability to distinguish which RNAs are to be degraded by the RNAi machinery or process. Included within the term small RNA are “small interfering RNAs” and “microRNA.” In general, microRNAs (miRNAs) are small (e.g., 17-26 nucleotides), single-stranded noncoding RNAs that are processed from approximately 70 nucleotide hairpin precursor RNAs by Dicer. Small interfering RNAs (siRNAs) are of a similar size and are also non-coding, however, siRNAs are processed from long dsRNAs and are usually double stranded. siRNAs can also include short hairpin RNAs in which both strands of an siRNA duplex are included within a single RNA molecule. Small RNAs can be used to describe both types of RNA. These terms include double-stranded RNA, single-stranded RNA, isolated RNA (partially purified RNA, essentially pure RNA, synthetic RNA, recombinantly produced RNA), as well as altered RNA that differs from naturally occurring RNA by the addition, deletion, substitution and/or alteration of one or more nucleotides. Such alterations can include addition of non-nucleotide material, such as to the end(s) of the small RNA or internally (at one or more nucleotides of the RNA). Nucleotides in the RNA molecules of the present invention can also comprise non-standard nucleotides, including non-naturally occurring nucleotides or deoxyribonucleotides. See “nucleobase oligomers” above for additional modifications to the nucleic acid molecule. In a preferred embodiment, the RNA molecules contain a 3′ hydroxyl group.

By “sepsis” is meant a disorder or state characterized by a source of infection, proven, for example, by a positive blood culture for a source of infection (or inferred on clinical grounds) accompanied by two or more of the following: a heart rate greater than 90 beats per minute; a body temperature less than 36° C. or 96.8° F. or greater than 38° C. or 100.4° F.; hyperventilation (high respiratory rate) greater than 20 breaths per minute or on blood gas a PₐCo₂ less than 32 mm Hg; and a white blood cell count<4000 cells/mm³ or >12000 cells/mm³ (<4×10⁹ or >12×10⁹ cells/L), or greater than 10% band forms (immature white blood cells).

By “specifically binds” is meant a compound or antibody which recognizes and binds a polypeptide of the invention but that does not substantially recognize and bind other molecules in a sample, for example, a biological sample, which naturally includes a polypeptide of the invention. In one example, an antibody that specifically binds Ang-2 does not specifically bind Ang-1.

By “subject” is meant a mammal, including, but not limited to, a human or non-human mammal, such as a bovine, equine, canine, ovine, or feline.

By “substantially identical” is meant a nucleic acid or amino acid sequence that, when optimally aligned, for example using the methods described below, share at least 60%, 65%, 70%, 75%, 80%, 85%, 90%, 95%, 96%, 97%, 98%, 99%, or 100% sequence identity with a second nucleic acid or amino acid sequence, e.g., an Ang-2 sequence. “Substantial identity” may be used to refer to various types and lengths of sequence, such as full-length sequence, epitopes or immunogenic peptides, functional domains, coding and/or regulatory sequences, exons, introns, promoters, and genomic sequences. Percent identity between two polypeptides or nucleic acid sequences is determined in various ways that are within the skill in the art, for instance, using publicly available computer software such as Smith Waterman Alignment (Smith and Waterman J Mol Biol 147:195-7, 1981); “BestFit” (Smith and Waterman, in “Advances in Applied Mathematics,” pp. 482-489, 1981) as incorporated into GeneMatcher Plus™, Schwarz and Dayhof “Atlas of Protein Sequence and Structure,” Dayhof, M. O., Ed pp 353-358, 1979; BLAST program (Basic Local Alignment Search Tool; Altschul et al. J. Mol. Biol. 215:403-10, 1990), BLAST-2, BLAST-P, BLAST-N, BLAST-X, WU-BLAST-2, ALIGN, ALIGN-2, CLUSTAL, or Megalign (DNASTAR) software. In addition, those skilled in the art can determine appropriate parameters for measuring alignment, including any algorithms needed to achieve maximal alignment over the length of the sequences being compared. In general, for proteins, the length of comparison sequences will be at least 10 amino acids, preferably 20, 30, 40, 50, 60, 70, 80, 90, 100, 110, 120, 130, 140, 150, 200, 250, 300, 350, 400, 450, or 500 amino acids or more. For nucleic acids, the length of comparison sequences will generally be at least 25, 50, 100, 125, 150, 200, 250, 300, 350, 400, 450, 500, 550, or at least 600, 700, 800, 900, 1000, 1100, 1200, 1300, 1400, or 1500 nucleotides or more. It is understood that for the purposes of determining sequence identity when comparing a DNA sequence to an RNA sequence, a thymine nucleotide is equivalent to a uracil nucleotide. Conservative substitutions typically include substitutions within the following groups: glycine, alanine; valine, isoleucine, leucine; aspartic acid, glutamic acid, asparagine, glutamine; serine, threonine; lysine, arginine; and phenylalanine, tyrosine.

By “therapeutic amount” is meant an amount that when administered to a subject suffering from any of the disorders of the invention (e.g., vascular leak or hypotension caused, for example, by sepsis or IL-2 therapy) is sufficient to cause a qualitative or quantitative reduction in the symptoms associated with the vascular leak disorder, for example, as described below.

By “treating” is meant administering a compound or a pharmaceutical composition for prophylactic and/or therapeutic purposes or administering treatment to a subject already suffering from a disease to improve the subject's condition or to a subject who is at risk of developing a disease. By “treating a vascular leak disorder” is meant that the disease and the symptoms associated with the disease are alleviated, reduced, cured, or placed in a state of remission. More specifically, when Ang-2 antagonist compounds, or fragments or derivatives thereof, are used to treat a subject with a vascular leak disorder, it is generally provided in a therapeutically effective amount to achieve any one or more of the following: reduce mortality, reduce vascular leakage, restore the integrity of vessel walls, prevent requirement for mechanical ventilation, reduce organ damage, increase in arterial blood pressure, increase in cardiac output, decreased systemic vascular resistance, decrease in the number of vasopressor medications necessary to maintain tissue perfusion, reduction in edema—bedside clinical assessment, increased urine output, decreased weight gain upon administration of intravenous fluids, increase in oxygenation of blood—increased PaO2/FiO2, increased oxygen saturation (SpO2), decreased positive end-expiratory pressure (PEEP) needed to ventilate lungs adequately, fall in respiratory rate, decrease in time to discontinuing mechanical ventilation, decrease in number of ICU days required, and decrease in time to resolution of shock.

By “vascular leak” is meant the movement of blood cells and fluid from the blood vessels into the surrounding tissues, including the lungs. Symptoms of vascular leak include reduced blood pressure, reduced cardiac output, increased systemic vascular resistance, edema, decreased urine output, decreased blood oxygenation, increase in respiratory rate, and shock. By “vascular leak disorder,” “vascular leak syndrome,” or “capillary leak syndrome” is meant any disorder that is characterized by a vascular leak or has associated with it the presence of a vascular leak. Vascular leak disorders include any of the following disorders that have associated vascular leak: sepsis (e.g., mild or severe); pneumonia; ALI; ARDS (e.g., transplant ARDS and ARDS due to burns, pancreatitis and trauma); vascular leak associated with drug therapy (e.g., IL-2, rituximab and others); idiopathic capillary leak syndromes; pre-eclampsia; eclampsia; hypotensive states due to sepsis; heart failure; trauma; infection; pulmonary aspiration of stomach contents; pulmonary aspiration of water; near drowning; burns; inhalation of noxious fumes; fat embolism; blood transfusion (TRALI, transfusion-related acute lung injury); amniotic fluid embolism; air embolism; edema; organ failure; poisoning; radiation; and inflammatory states (e.g., acute and chronic vascular rejection, pancreatitis, trauma, and vasculitis). Less common etiologies of vascular leak include genetic disorders that intermittently produce vascular leak (e.g. C1 esterase inhibitor deficiency or familial fever syndromes such as TRAPS—TNF receptor associated periodic fever syndrome), massive blood transfusion, anaphylaxis or similar hypersensitivity reactions; post-lung or post-heart-lung transplant; and ovarian hyperstimulation syndrome (e.g., as described in Garcia-Velasco et al. Curr Opin Obstet. Gynecol. 15:251-256 (2003)). Vascular leak can also be caused by VEGF or bradykinin overexpression.

By “vector” is meant a DNA molecule, usually derived from a plasmid or bacteriophage, into which fragments of DNA may be inserted or cloned. A recombinant vector will contain one or more unique restriction sites, and may be capable of autonomous replication in a defined host or vehicle organism such that the cloned sequence is reproducible. A vector contains a promoter operably linked to a gene or coding region such that, upon transfection into a recipient cell, an RNA is expressed.

By “VEGF inhibitor compound” is meant any compound that reduces or inhibits the expression or biological activity of VEGF. Non-limiting examples include anti-VEGF antibodies and VEGF tyrosine kinase inhibitors such as Lucentis and Avastin (ranibizumab and bevacizumab, Genentech), PTK787/ZK222584 (Novartis), SU5416, AZD 2171, ZD6474 (Zactima), AZD9935 (AstraZeneca), sorafenib or 43-9006 (Bayer), and sutent SU011248 (Pfizer).

Other features and advantages of the invention will be apparent from the following Detailed Description, the drawings, and the claims.

### BRIEF DESCRIPTION OF THE DRAWINGS

The application file contains drawings executed in color (FIGS. 4A-4U, 5A (panels a-f), 6E, 7C, 8B, 9A-C, 14A-B, 15C, 17B, 17D, 19E-F, and 21A-B). Copies of this patent or patent application with color drawings will be provided by the Office upon request and payment of the necessary fee.

FIG. 1 is a graph showing serum Ang-2 levels at study enrollment.

FIG. 2 is a graph showing the temporal trends of circulating Ang-2 in three illustrative study subjects. Patient CH (-▪-), a 74-year-old woman, was admitted to the medical intensive care unit with severe sepsis. She was treated with broad-spectrum antibiotics, initially required three vasoactive agents to manage shock, and was mechanically ventilated. Patient CH's nadir PaO₂/FiO₂=240 occurred on hospital day 2, correlating with her peak circulating Ang-2. Enterococcus was grown from her urine. She progressively convalesced and was extubated prior to discharge. Patient AP (-▴-), a 92-year-old woman, was admitted to the general medicine service from a nursing home for increased confusion over her baseline dementia. She had no evidence of sepsis, shock, or respiratory compromise—PaO₂/FiO₂>300. She was treated for a foot wound infection with two antibiotics and was discharged in stable condition back to the nursing home. Patient AG (-∘-), a 77-year-old man, was first admitted to the general medicine service with hypotension following excessive fluid removal at hemodialysis—there was no evidence of infection, systemic inflammatory response, or respiratory compromise with PaO₂/FiO₂>300 (hospital days 1-3). However, three months later (graphed as hospital days 6-8 for purposes of illustration), the same patient (-∘-) was re-admitted to the intensive care unit following emergent right leg amputation for gangrene complicated by shock and inability to extubate. Nadir PaO₂/FiO₂=144 occurred on the same day as peak Ang-2 (depicted as hospital day 8), when he died despite full care.

FIG. 3 is a graph showing peak circulating Ang-2 correlates with impaired pulmonary gas exchange. Impaired oxygenation of blood, as assessed by the nadir PaO₂/FiO₂ ratio, correlates with significant differences in circulating Ang-2, *p=0.0195.

FIGS. 4A-4U are a series of photomicrographs showing the effects of serum from human subjects with sepsis on endothelial architecture. Ten percent FBS or 10% serum from one of two patients with sepsis was incubated with endothelial cell (EC) monolayers to assess effects on endothelial architecture. High Ang-2 serum (Patient CE4, Ang-2=89 ng/ml) induced thick actin stress fibers and intercellular gap formation (FIGS. 4D-4F), whereas low Ang-2 serum (CF1, Ang-2=8.9 ng/ml) did not (FIGS. 4G-4I). The gap-promoting effect of Patient CE4's serum was reversed with addition of 100-ng/ml recombinant human Ang-1 (FIGS. 4J-4L) and was indistinguishable from control cells that exhibit thin actin fibers and no intercellular gaps (FIGS. 4A-C). Serum was then taken from one patient (Patient CG), drawn on hospital day 2 (Patient CG2, Ang-2=78 ng/ml) and hospital day 16 (Patient CG12, Ang-2=6.3 ng/ml), and was added at 10% to HMVEC monolayers. Again, high-Ang-2 serum (Patient CG2) induced gap formation and thick actin stress fibers (FIGS. 4M-40), effects not seen in the serum of with the same patient's serum at discharge (Patient CG12) (FIGS. 4P-4R) and effects that were reversed with the addition of 100 ng/ml Ang-1 (FIGS. 4S-4U). Arrows indicate intercellular gaps.

FIG. 5A, panels a-f, are a series of photomicrographs showing the addition of Ang-2 alone to EC monolayers disrupts endothelial architecture at physiologic concentrations. (A) Control (vehicle) or recombinant human Ang-2 (100 ng/ml) was added to HMVEC monolayers. These cells were then fixed and stained for F-actin and VE-cadherin. Shown are healthy control cells (panels a-c) versus Ang-2 treated cells (panels d-f), which exhibit thick actin stress fibers and disrupted junctions, leaving intercellular gaps (arrows).

FIG. 5B is a graph showing the Pₐ for HMVECs treated with vehicle or Ang-2. HMVECs were grown to confluence on Transwell membranes coated with fibronectin. Monolayers were treated with vehicle or Ang2 (400 ng/ml in luminal chamber) plus FITC-albumin. Permeability of albumin (Pₐ) was calculated after 8 hours as described in the Methods section. Pₐ values are expressed as percentage of control cells. *p<0.01.

FIGS. 6A-6D are a series of autoradiograms showing the effect of Ang-2 on Rho kinase and myosin light chain kinase activation. In FIG. 6A, serum was taken from two patients—Patient CE2 (Ang-2=77 ng/ml) and Patient CF5 (Ang-2=7.9 ng/ml)—and added at 20-fold dilution to 24 hour serum-starved HMVECs. High Ang-2-serum (Patient CE2) caused MLC phosphorylation that was diminished by addition of Ang-1 (100 ng/ml), whereas low Ang-2-serum (CF5) did not induce robust MLC phosphorylation. After 24-h serum starvation, Ang-2 (100 ng/ml) was added to HMVECs, and cells were lysed at the indicated times. FIG. 6B is a western blot showing MLC phosphorylation (MLC-p). MLC-p was elevated at 3 hours and 6 hours of stimulation. After 24 hours serum starvation, Ang-2 (100 ng/ml) was added to HMVECs, and cells were lysed at the indicated times. FIG. 6C is a western blot showing GTP-RhoA expression peaked at 30 to 60 minutes of Ang-2 stimulation. After 24 hours serum starvation, HMVECs were stimulated with Ang-2 (100 ng/ml) with or without 10 M Y27632 (Rho-kinase inhibitor) or 10 M ML-7 (MLCK inhibitor) for 5 hours. FIG. 6D is a western blot showing MLC-p phosphorylation. Y27632 had a more potent inhibitory effect on MLC-p than equimolar ML-7.

FIG. 6E (panels a-i) is a series of photomichrographs showing that Ang-2 promotes stress fibers within cells and gap formation between cells. HMVECs were grown to confluence and incubated for 5 hours with Ang-2 (100 ng/ml) (panels a-c). Stress fibers can be seen in panel a and gap formation is shown by the arrows in panel c. HMVECs were also stimulated with Ang-2 (100 ng/ml) in the presence of 10 M Y27632 (panels d-f) or 10 M ML-7 (panels g-i). Co-incubation with Y27632 or ML-7 reversed the effects of Ang-2 on stress fibers and gap formation. Cells were fixed and stained for F-actin and VE-cadherin as described in the Methods section. Shown are representative confocal fluorescence microscopy images (600xx). F-actin-panels a, d, and g; VE-cadherin-panels b, e, and h; merge images-panels c, f, and i.

FIG. 7A shows two autoradiograms showing Tie-2 phosphorylation in HMVECS stimulated with Ang-2 (100 ng/ml) in 2.5% FBS EBM-2 for the indicated times. Phospho-Tie-2 was detected by immunoprecipitation and western blot (upper bar) as described in Methods. Similar amounts of total Tie-2 were present in HMVECs harvested at each time point. Phospho-Tie-2 declined over time whereas total Tie-2 remained relatively constant (lower bar).

FIG. 7B shows three autoradiograms showing the effects of a Tie-2 knock-down on MLC-p. Negative control siRNA (left column) or a Tie-2-specific siRNA (right column) was transfected in HMVECs. Cells were then serum-starved for 24 hours, after which decreased Tie-2 expression (right, upper) and increased MLC phosphorylation (right, middle) were verified with Tie-2 siRNA.

FIG. 7C is a series of photomicrographs showing phase contrast (200xx) and fluorescence images (600xx) of cells stained for F-actin and VE-cadherin after transfection of negative control siRNA (panels a-d) or Tie-2 specific siRNA (panels e-h). Tie-2-siRNA caused thick actin stress fibers and gap formation in HMVECs (panel h, arrows). Phase contrast images, panels a and e; F-actin, panels b and f; VE-cadherin, panels c and g; merge images, panels d and h.

FIG. 8A is a graph showing the leakage of Evans blue out of the vasculature and into the lung and liver of Ang-2 treated mice. After injection of vehicle or Ang-2 (10 g, intraperitoneal), mice were injected in the retro-orbital sinus with Evans blue (2%, 50 l); after sacrifice, intravascular Evans blue was washed out with PBS and vascular leakage was evaluated by quantifying extravasated Evans blue. The amount of Evans blue in organ homogenates was spectrophotometrically quantified. Evans blue content significantly increased in the lung and liver of Ang-2-treated mice, indicating leakage out of the vasculature and impregnation within the tissue, *p<0.01.

FIG. 8B shows two representative photographs of lungs taken after washout of intravascular Evans blue with PBS (phosphate-buffered saline) for 10 minutes. The lung from a control (vehicle intraperitoneal) mouse (left) appears blanched in contrast to the purple-tinted, congested lung from an Ang-2-treated mouse (right).

FIG. 8C is a graph showing the lung wet-to-dry (W/D) weight ratio. Ang-2 treatment for 16 hours increased lung W/D weight ratio, consistent with congestion due to water accumulation, *p<0.01.

FIGS. 9A-9C is a series of photomicrographs showing systemic Ang-2 administration provokes rapid and progressive pulmonary congestion. Ang-2 was administered intraperitoneally (10 g), and lung sections were assessed for histologic changes. Control lung is shown at 100xx in FIG. 9A. Note the thin alveolar septa, particularly in the inset (400xx). FIG. 9B shows the lung 3 hours after Ang-2, where there is noticeable expansion of alveolar septa with increase in cellularity, reduction in air-space, and some leakage of cells into the alveolar space. FIG. 9C shows the advancement of the changes after 2 days of systemic Ang-2 administration (total dose 20 g).

FIG. 10 is a western blot showing Ang-2 stimulated MLC-phosphorylation (MLC-p) is inhibited by an inhibitor of NF-kB (panepoxydone).

FIG. 11 shows two graphs showing the levels of Ang-2 in 4 subjects pre and post-IL-2 infusion (top) and the levels of Ang-2 after serial serum measurement during and after five consecutive days of daily IL-2 infusions.

FIG. 12 is an autoradiogram of two western blots showing serum Ang-2 rises after cecal ligation and perforation (CLP) but not sham operation.

FIGS. 13A-E is a series of autoradiograms showing Ang-1 has opposite effects on Rac1 and RhoA through p190RhoGAP. FIG. 13A shows Ang-1 activates Rac1 and inactivates RhoA. HMVEC-L were incubated with Ang-1 and cells were lysed at the indicated times. GTP-bound active form of Rac1 was collected by PAK pull-down assay and detected by immunoblotting with anti-Rac antibody. The GTP-bound active form of RhoA was collected by rhotekin pull-down assay and detected by immunoblotting with anti-Rho antibody. FIG. 13B shows PI3K inhibition blocks Ang-1 induced Rac1 activation. HMVEC-L were incubated with Ang-1 with or without PI3K inhibitor, LY294002 (10 μM) for 30 min. GTP-bound active Rac1 was detected as described for FIG. 13A. FIG. 13C shows active Rac1 is necessary for Ang-1 to inhibit RhoA. Dominant negative Rac1T17N was delivered using a lentivirus vector. HMVEC-L were incubated with Ang-1, and Rac and Rho activity were measured as described above. FIG. 13D shows Ang-1 induces phosphorylation of p190RhoGAP in a Rac1-dependent fashion. p190RhoGAP phosphorylation was detected in vehicle (Cont) and Ang-1 (100 ng/ml) treated HMVEC-L (left panel). HMVEC-L transfected with Rac1T17N lentivirus were treated with control (Cont) or Ang-1 (100 ng/ml) for 30 minutes (right panel). FIG. 13E, panels a and b show p190RhoGAP is not required for Ang-1 mediated Rac1 activation but is necessary for Ang-1 to suppress RhoA. p190RhoGAP knockdown does not block Ang-1-induced Rac1 activation but does block Ang-1-induced RhoA inactivation. siRNA against p190RhoGAP was transfected as described in Methods. p190RhoGAP expression in HMVEC-L transfected with siRNA is shown in panel a. GAPDH is blotted as a loading control. Panel b shows GTP-bound Rac1 and RhoA levels from HMVEC-L incubated with vehicle (control) or Ang-1 for 30 minutes. Note: n=4 per group.

FIGS. 14A-B are photomicrographs showing Ang-1-induced fortification of cell boundaries requires PI3K activation, Rac1 activation, and RhoA suppression. FIG. 14A panels a-l show Ang-1 causes peripheral MLC-P and cortical actin rearrangement in a PI3K-dependent manner. Confluent monolayers of HMVEC-L were incubated with vehicle (control) or Ang-1 with or without PI3K inhibitor, LY294002 (10 μM), in 0.25% FBS EBM-2 for 30 min. The cells were then fixed and stained for F-actin (red, panels a, e, i), MLC-P (green, panels b, f, j), nucleus (blue) and VE-cadherin (green, panels d, h, 1). Shown are control (a-d), Ang-1 (e-h), Ang-1 plus LY294002 (i-l). White arrows indicate intercellular gap formation. Scale bar, 5 μm. For FIG. 14B panels a-f Rac1T17N (dominant negative Rac1) or RhoAG14V (constitutively active RhoA) was delivered to cells using lentiviral vectors. After reaching confluency, HMVEC-L were incubated with vehicle (Control, panels a-c) or Ang-1 (panels d-f) in 0.25% FBS/EBM-2 for 30 minutes. The cells were then fixed and stained for VE-cadherin. Shown are cells transfected with control virus (panels a, d), Rac1T17N (panels b, e), or RhoAG14V (panels c, f). Scale bar, 5 μm.

FIGS. 15A-C shows Ang-1 reverses endotoxin-induced Rac1 and RhoA signaling and requires active Rac1 and p190RhoGAP to block endotoxin-induced endothelial structural distortion. FIG. 15A is a series of autoradiograms and graphs showing Rac and Rho activity in HMVEC-L cells. HMVEC-L cells were stimulated with endotoxin (LPS 100 ng/ml) for 30 minutes with or without Ang-1. Cells were lysed and Rac and Rho activity were measured. p<0.01, ** p<0.05. Mean±SEM of four experiments. FIG. 15B is a series of autoradiograms showing Rho activity after incubation with p190RhoGAP siRNA. siRNA was added to HMVEC-L and then the cells were stimulated with endotoxin (LPS 100 ng/ml) for 30 minutes with or without Ang-1 and Rho activity was measured. FIG. 15C (panels) a-e is a series of photomicrographs showing HMVEC-L cells treated with control virus (panels a-c), Rac1T17N (panel d), or p190RhoGAP siRNA (panel e). Delivery of Rac1T17N lentivirus or transfection with p190RhoGAP siRNA was performed as described herein. After reaching confluency, HMVEC-L were incubated with vehicle (panel a), endotoxin alone (LPS 100 ng/ml, panel b), or endotoxin and Ang-1 (panels c-e) in 0.25% FBS/EBM-2 for 30 minutes. The cells were then fixed and stained for VE-cadherin. Shown are cells transfected with (Note: control siRNA cells treated with endotoxin and Ang-1 were indistinguishable from panel c). White arrows indicate intercellular gap formation. Scale bar, 5 μm.

FIGS. 16A-C are a series of graphs showing inhibition of PI3K, Rac1, or p190RhoGAP is sufficient to abrogate the protective effect of Ang-1 against endotoxin on endothelial permeability. In FIG. 16A, HMVEC-L were grown to confluence on Transwell membranes coated with 0.5% gelatin. Cells were treated with vehicles, Ang-1 (100 ng/ml), LPS, LPS with Ang-1, or LPS with Ang-1 and the PI3K inhibitor, LY290042 (10 μM). Permeability was evaluated after 4 hours. Pa values are expressed as percentage of control cells (See Methods for calculation), * p<0.01. In FIG. 16B, HMVEC-L were transfected with Rac1T17N lentivirus and subjected to the permeability assay as described for FIG. 16A. *p<0.01. In FIG. 16C, HMVEC-L were transfected with p190RhoGAP siRNA and subjected to the permeability assay as described above. *p<0.01. Mean±SEM of four experiments.

FIGS. 17A-D show Ang-1 blocks LPS-induced pulmonary hyperpermeability in vivo in a p190RhoGAP-dependent fashion. FIG. 17A is a graph showing lung permeability (Measured in absorbance units) in control mice (vehicle ip), endotoxin (LPS 100 μg ip) or endotoxin plus Ang-1 (10 μg ip×2 doses). FIG. 17B is a series of photomicrographs showing H and E stained 40× photomicrographs of lungs taken from animals treated as in (FIG. 17A) above. LPS results in edema and leukocyte infiltration that are reversed by Ang-1. FIG. 17C, panel a, is an autoradiogram showing p190RhoGAP protein levels are reduced in mouse lung after hydrodynamic delivery of specific siRNA but not after delivery of control siRNA. FIG. 17C, panel b, is a graph showing in mice treated with control siRNA, endotoxin-induced permeability was unaffected as was the rescue ability of Ang-1; p190RhoGAP knockdown blocked the anti-permeability effect of Ang-1 in vivo (Note: p190siRNA treatment in the absence of endotoxin caused no change in basal lung permeability compared to control siRNA). FIG. 17D is a series of H and E stained 40× photomicrographs of lungs taken from animals treated as in FIG. 17C above. In the presence of p190RhoGAP knockdown, Ang-1 can no longer inhibit endotoxin-induced edema and inflammation. *p<0.05. Mean±SEM of four experiments.

FIG. 18 is a schematic showing the proposed Ang-1 barrier-protective signaling. The lower right scheme illustrates the counteracting effects of Rac1 and RhoA on actin, myosin light chain (MLC-P), and VE-cadherin at endothelial junctions. Either the Rac1 or RhoA limb is activated with a particular stimulus. p190RhoGAP links Rac1 activation to RhoA inhibition to coordinate these cytoskeletal regulators. We demonstrated that excess Ang-1 (upper left cell) shifts the balance between Rac1 and RhoA towards Rac1, leading to enhanced peripheral MLC-P, cortical actin rearrangement, and augmentation of the junctional VE-cadherin. Endotoxin, on the other hand, activates RhoA and shifts the balance away from Rac1, destabilizing cell architecture with red central actin stress fibers and “unzipped” green VE-cadherin resulting in interendothelial gaps and subsequent permeability. Upon coincubation of Ang-1 and endotoxin, the barrier protective effect of Ang-1 predominates by directly augmenting Rac1 activity as well as by suppressing RhoA activity through p190RhoGAP. We have shown that loss of PI3K, Rac1, or p190RhoGAP is sufficient to abrogate the protective effect of Ang-1 against endotoxin.

FIGS. 19A-F show Ang-1 induces phosphorylation of Tie2, the p85 subunit of PI3K, and Akt, and increases MLC-P in HMVEC-L. FIG. 19A is an autoradiogram showing that Ang-1 induces Tie2 phosphorylation in HMVEC-L. Human lung microvascular endothelial cells (HMVEC-L) were incubated with vehicle (Cont) or Ang-1 (100 ng/ml) for 15 minutes. Phosphorylated Tie-2 was immunoprecipitated with anti-Tie-2 antibody and detected by immunoblot with anti-p-Tyr antibody as described herein. IP, immunoprecipitation; IB, immunoblot. FIG. 19B is a graph showing that Ang-1 induces phosphorylation of the p85 subunit of PI3K. HMVEC-L were incubated with vehicle (Cont) or Ang-1 (100 ng/ml) for 15 minutes and fixed. Total phosphorylation of the p85 subunit of PI3K was measured in triplicate by a commercial enzyme-linked immunosorbent assay as described herein. Data was plotted after correction with total PI3K. *P<0.01. FIG. 19C is an autoradiogram showing that Ang-1 induces Akt phosphorylation. HMVEC-L were incubated with vehicle (Cont) or Ang-1 (100 ng/ml) for 15 minutes. Total Akt and phosphorylated Akt were detected by Western blot. FIG. 19D is an autoradiogram showing that Ang-1 increases MLC-P in HMVEC-L. HMVEC-L were incubated with Ang-1 for the indicated times and MLC-P was detected by immunoblotting. FIG. 19E is a photomicrograph showing HA staining for lentiviral infection. Lentiviral delivery of Rac1T17N or RhoAG14V was performed as described herein. The cells were then fixed and stained for HA, scale bar, 5 μm. FIG. 19F, panels and b, are photomicrographs showing that knockdown of p190RhoGAP does not prevent the ability of Ang-1 to fortify cell boundaries. p190RhoGAP siRNA and VE-cadherin staining (green) were performed as described herein. Confluent monolayers of HMVEC-L were incubated with vehicle (Control, panel a) or Ang-1 (100 ng/ml, panel b) in 0.25% FBS EBM-2 for 30 minutes. Scale bar, 5 μm.

FIG. 20A is a graph showing the levels of Ang 2 in fourteen patients in blood as measured at baseline, their peak value following administration of IL-2, and one day following the last dose of IL-2. In three patients, figures are shown for data obtained two days after cessation of IL-2 therapy.

FIG. 20B is a graph showing Ang 2 levels in blood in patient #8, showing a detailed time course during IL-2 administration.

FIG. 21A is a series of confocal images showing actin, left hand column, VE cadherin, middle column, and merged, right hand column for four conditions when patient blood is added to an endothelial confluent monolayer. The top row shows data obtained with controls indicating relatively sparse actin staining and marked junctional VE cadherin staining. The second row shows data obtained from patient 11 at day 1, with an Ang 2 level of 3.9 ng/mL, which looks similar to controls. However, blood from the same patient on day 5 (third row), when the Ang 2 level was 41.8, clearly shows increased actin fibers and markedly diminished VE cadherin staining, as well as the presence of intercellular gaps. The last row shows the data obtained when Ang-1, at a level of 100 ng/mL, has been added to the blood from patient 11 30 minutes after the start of incubation. It is clear that the monolayer has been restored with a decrease in actin staining, an increase in junctional VE cadherin and the loss of gaps—reminiscent of control serum.

FIG. 21B is a series of photomicrographs showing data from patient 10 when his Ang 2 level was 52.6 (top row) with the results of the Ang 1 rescue experiment shown in the bottom row.

FIG. 22A is a graph showing the serial measurements of VEGF in blood of patients treated with high dose IL-2 in the absence of bevacizumab, a neutralizing antibody to VEGF. These measurements are shown for a total of 8 patients during their hospital stay when IL-2 was administered.

FIG. 22B, top, shows characteristics of the four patients who received high dose IL-2 with bevacizumab. The left hand lower graph shows VEGF levels in these patients during their hospital course. As expected pre-VEGF levels are essentially 0 because these patients have all received bevacizumab. The right hand graph shows Ang 2 levels in these patients during their hospital course, showing that Ang 2 levels rise during the hospital course in a manner similar to what happens in a larger set of patients when no bevacizumab is used.

FIG. 23A shows the amino acid sequence for human Ang-2 (SEQ ID NO: 1).

FIG. 23B shows the nucleic acid sequence for human Ang-2 (SEQ ID NO: 2). FIG. 24A shows the amino acid sequence for human Ang-1 (SEQ ID NO: 3).

FIG. 24B shows the nucleic acid sequence for human Ang-1 (SEQ ID NO: 4).

### DETAILED DESCRIPTION

Ang-1 and Ang-2 are peptide ligands that bind the Tie-2 receptor tyrosine kinase found primarily on endothelial cells (ECs). They were first identified as an agonist/antagonist pair necessary for embryonic vascular development. Ang-1 appears to promote vessel stability by recruiting pericytes to nascent blood vessels and preserving cell-cell contacts. Ang-1, expressed in supraphysiologic concentrations, appears to function as an anti-permeability agent in rodent dermal capillaries. However, there are no previous reports describing an increase in Ang-2 levels or an imbalance in Tie-2 receptor signaling in the development of vascular leak syndrome under physiological conditions. In addition, none of these reports have demonstrated a role for Ang-2 and the Ang-2 signaling pathway in the pathogenesis of vascular leak syndromes. Our discoveries, using human serum from patients suffering from vascular leak syndrome, are the first to demonstrate the pathogenic role for Ang-2 in vascular leak syndrome under physiological conditions. Furthermore, our results are the first to demonstrate an elevation in Ang-2 levels in serum from patients suffering from vascular leak syndrome. No significant difference in Ang-1 levels was detected in these patients.

We have discovered that Ang-2 is both a marker for and a mediator of vascular leak syndromes and hypotension, including sepsis, ARDS, ALI, and IL-2 therapy associated vascular leak. We have shown that Ang-2 levels are elevated in patients with vascular leak syndrome and impairment in gas exchange and that measurement of Ang-2 levels can be used as a tool to diagnose or predict the prognosis of a subject having or at risk for sepsis or any other disorders characterized by vascular leak, hypotension, or a procoagulant state. We have also shown that Ang-2 acts to distort endothelial cell architecture and produce vascular leak and pulmonary injury, at least in part, through binding to Tie-2 and activation of myosin light chain phosphorylation via Rho kinase. This discovery is supported by the findings described herein demonstrating that Ang-1 regulates the endothelial cytoskeleton and protects against vascular leak by shifting the GTPase balance through dual actions that activate Rac1 through PI3K and inhibit RhoA through p190RhoGAP. Antagonists to Ang-2 (e.g., any compound that reduces Ang-2 levels or blocks Ang-2 activity) or any signaling proteins activated downstream of Ang-2 are also useful as therapeutics for the treatment or prevention of disorders that are characterized by vascular leak, hypotension, or a procoagulant state. Examples of such disorders are described below and include sepsis, ARDS, ALI, and vascular leak syndrome associated with high dose interleukin-2 (HD IL-2) therapy.

### Vascular Leak Syndromes

Blood vessels are normally lined with tightly linked cells, called endothelial cells that form an impermeable barrier. Vascular leak occurs when small blood vessels, generally a capillary or venule, become leaky and release fluid. There are many diseases and even some therapeutic regimens that are associated with vascular leak and these are all included as vascular leak syndromes for the purposes of the present invention. Non-limiting examples of vascular leak syndromes are described above and sepsis (e.g., mild or severe); pneumonia; ALI; ARDS (e.g., transplant ARDS and ARDS due to burns, pancreatitis and trauma); vascular leak associated with drug therapy (e.g., IL-2, rituximab and others); idiopathic capillary leak syndromes; pre-eclampsia; eclampsia; hypotensive states due to sepsis; heart failure; trauma; infection; pulmonary aspiration of stomach contents; pulmonary aspiration of water; near drowning; burns; inhalation of noxious fumes; fat embolism; blood transfusion (TRALI, transfusion-related acute lung injury); amniotic fluid embolism; air embolism; edema; organ failure; poisoning; radiation; inflammatory states (e.g., acute and chronic vascular rejection, pancreatitis, trauma, and vasculitis); genetic disorders that intermittently produce vascular leak (e.g. C1 esterase inhibitor deficiency or familial fever syndromes such as TRAPS—TNF receptor associated periodic fever syndrome); massive blood transfusion; anaphylaxis or similar hypersensitivity reactions; post-lung or post-heart-lung transplant; and ovarian hyperstimulation syndrome.

### Therapeutics

We have discovered that Ang-2 in human sepsis serum is responsible for endothelial distortion and this activity dissipates with clinical resolution, and is reversed by Ang-1. We have also discovered that this endothelial distortion effect resulting in endothelial barrier disruption is mediated by MLC phosphorylation in ECs and that Ang-1 regulates the endothelial cytoskeleton and protects against vascular leak by shifting the GTPase balance through dual actions that activate Rac1 through PI3K and inhibit RhoA through p190RhoGAP. We have also discovered that systemic administration of Ang-2 to healthy adult mice provokes rapid and severe pulmonary vascular leak and congestion. In sum, these results demonstrate a role for Ang-2 and downstream Tie-2 signaling proteins in the pathogenesis of vascular leak syndrome.

Accordingly, the invention features the use of therapeutic compounds that function as Ang-2 antagonists. Ang-2 antagonists include any synthetic or natural polypeptide, nucleic acid, or small molecule compound that can decrease the levels of Ang-2 or reduce or block Ang-2 signaling either by affecting Ang-2 directly or by affecting downstream effector molecules of Ang-2 signaling pathways. Non-limiting examples of therapeutic compounds useful in the methods of the invention are described in detail below.

Therapeutics that Decrease the Levels of Ang-2

The present invention also features therapeutic nucleic acids that can be used to decrease the levels of Ang-2 for the treatment or prevention of vascular leak. Such therapeutic nucleic acids include antisense nucleobase oligomers or small RNAs to downregulate expression of Ang-2 mRNA directly.

By binding to the complementary nucleic acid sequence (the sense or coding strand), antisense nucleobase oligomers are able to inhibit protein expression presumably through the enzymatic cleavage of the RNA strand by RNAse H. Preferably the antisense nucleobase oligomer is capable of reducing Ang-2 protein expression in a cell that expresses increased levels of Ang-2. Preferably the decrease in Ang-2 protein expression is at least 10% relative to cells treated with a control nucleobase oligomer, preferably 20% or greater, more preferably 40%, 50%, 60%, 70%, 80%, 90% or greater. Methods for selecting and preparing Ang-2 antisense nucleobase oligomers are well known in the art. For an example of the use of antisense nucleobase oligomers to downregulate VEGF expression see U.S. Pat. No. 6,410,322, incorporated herein by reference. Methods for assaying levels of protein expression are also well known in the art and include western blotting, immunoprecipitation, and ELISA.

One example of an antisense nucleobase oligomer particularly useful in the methods and compositions of the invention is a morpholino oligomer. Morpholinos are used to block access of other molecules to specific sequences within nucleic acid molecules. They can block access of other molecules to small (˜25 base) regions of ribonucleic acid (RNA). Morpholinos are sometimes referred to as PMO, an acronym for phosphorodiamidate morpholino oligo.

Morpholinos are used to knock down gene function by preventing cells from making a targeted protein or by modifying the splicing of pre-mRNA. Morpholinos are synthetic molecules that bind to complementary sequences of RNA by standard nucleic acid base-pairing. While morpholinos have standard nucleic acid bases, those bases are bound to morpholine rings instead of deoxyribose rings and linked through phosphorodiamidate groups instead of phosphates. Replacement of anionic phosphates with the uncharged phosphorodiamidate groups eliminates ionization in the usual physiological pH range, so morpholinos in organisms or cells are uncharged molecules.

Morpholinos act by “steric blocking” or binding to a target sequence within an RNA and blocking molecules which might otherwise interact with the RNA. Because of their completely unnatural backbones, morpholinos are not recognized by cellular proteins. Nucleases do not degrade morpholinos and morpholinos do not activate toll-like receptors and so they do not activate innate immune responses such as the interferon system or the NF-κB mediated inflammation response. Morpholinos are also not known to modify methylation of DNA. Therefore, morpholinos directed to any part of Ang-2 and that reduce or inhibit the expression levels or biological activity of Ang-2, by at least 10%, 20%, 30%, 40%, 50%, 60%, 70%, 80%, 90%, or more, are particularly useful in the methods and compositions of the invention that include Ang-2 antagonists.

The present invention also features the use of RNA interference (RNAi) to inhibit expression of Ang-2. RNAi is a form of post-transcriptional gene silencing initiated by the introduction of double-stranded RNA (dsRNA). Short 15 to 32 nucleotide double-stranded RNAs, known generally as “siRNAs,” “small RNAs,” or “microRNAs” are effective at down-regulating gene expression in nematodes (Zamore et al., Cell 101: 25-33) and in mammalian tissue culture cell lines (Elbashir et al., Nature 411:494-498, 2001, hereby incorporated by reference). The further therapeutic effectiveness of this approach in mammals was demonstrated in vivo by McCaffrey et al. (Nature 418:38-39. 2002). The small RNAs are at least 15 nucleotides, preferably, 17, 18, 19, 20, 21, 22, 23, 24, 25, 26, 27, 28, 29, 30, 31, 32, 33, 34, 35, nucleotides in length and even up to 50 or 100 nucleotides in length (inclusive of all integers in between). Such small RNAs that are substantially identical to or complementary to any region of Ang-2, are included in the invention.

Therefore, the invention includes any small RNA substantially identical to at least 15 nucleotides, preferably, 17, 18, 19, 20, 21, 22, 23, 24, 25, 26, 27, 28, 29, 30, 31, 32, 33, 34, or 35, nucleotides in length and even up to 50 or 100 nucleotides in length (inclusive of all integers in between) of any region of Ang-2. It should be noted that longer dsRNA fragments can be used that are processed into such small RNAs. Useful small RNAs can be identified by their ability to decrease Ang-2 expression levels or biological activity. Small RNAs can also include short hairpin RNAs in which both strands of an siRNA duplex are included within a single RNA molecule.

The specific requirements and modifications of small RNA are known in the art and are described, for example, in PCT Publication No. WO01/75164, and U.S. Application Publication Numbers 20060134787, 20050153918, 20050058982, 20050037988, and 20040203145, the relevant portions of which are herein incorporated by reference. In particular embodiments, siRNAs can be synthesized or generated by processing longer double-stranded RNAs, for example, in the presence of the enzyme dicer under conditions in which the dsRNA is processed to RNA molecules of about 17 to about 26 nucleotides. siRNAs can also be generated by expression of the corresponding DNA fragment (e.g., a hairpin DNA construct). Generally, the siRNA has a characteristic 2- to 3-nucleotide 3′ overhanging ends, preferably these are (2′-deoxy) thymidine or uracil. The siRNAs typically comprise a 3′ hydroxyl group. In some embodiments, single stranded siRNAs or blunt ended dsRNA are used. In order to further enhance the stability of the RNA, the 3′ overhangs are stabilized against degradation. In one embodiment, the RNA is stabilized by including purine nucleotides, such as adenosine or guanosine. Alternatively, substitution of pyrimidine nucleotides by modified analogs e.g. substitution of uridine 2-nucleotide overhangs by (2′-deoxy)thymide is tolerated and does not affect the efficiency of RNAi. The absence of a 2′ hydroxyl group significantly enhances the nuclease resistance of the overhang in tissue culture medium.

siRNA molecules can be obtained through a variety of protocols including chemical synthesis or recombinant production using a Drosophila in vitro system. They can be commercially obtained from companies such as Dharmacon Research Inc. or Xeragon Inc., or they can be synthesized using commercially available kits such as the Silencer™ siRNA Construction Kit from Ambion (catalog number 1620) or HiScribe™ RNAi Transcription Kit from New England BioLabs (catalog number E2000S).

Alternatively siRNA can be prepared using standard procedures for in vitro transcription of RNA and dsRNA annealing procedures such as those described in Elbashir et al. (Genes & Dev., 15:188-200, 2001), Girard et al., (Nature Jun. 4, 2006, e-publication ahead of print), Aravin et al., (Nature 442:203-207 (2006)), Grivna et al., (Genes Dev. 20:1709-1714 (2006)), and Lau et al., (Science 313:363-367 (2006)). siRNAs are also obtained by incubation of dsRNA that corresponds to a sequence of the target gene in a cell-free Drosophila lysate from syncytial blastoderm Drosophila embryos under conditions in which the dsRNA is processed to generate siRNAs of about 21 to about 23 nucleotides, which are then isolated using techniques known to those of skill in the art. For example, gel electrophoresis can be used to separate the 21-23 nt RNAs and the RNAs can then be eluted from the gel slices. In addition, chromatography (e.g. size exclusion chromatography), glycerol gradient centrifugation, and affinity purification with antibody can be used to isolate the small RNAs.

Short hairpin RNAs (shRNAs), as described in Yu et al. or Paddison et al. (Proc. Natl. Acad. Sci. USA, 99:6047-6052, 2002; Genes & Dev, 16:948-958, 2002; incorporated herein by reference), can also be used in the methods of the invention. shRNAs are designed such that both the sense and antisense strands are included within a single RNA molecule and connected by a loop of nucleotides (3 or more). shRNAs can be synthesized and purified using standard in vitro T7 transcription synthesis as described above and in Yu et al. (supra). shRNAs can also be subcloned into an expression vector that has the mouse U6 promoter sequences which can then be transfected into cells and used for in vivo expression of the shRNA.

A variety of methods are available for transfection, or introduction, of dsRNA into mammalian cells. For example, there are several commercially available transfection reagents useful for lipid-based transfection of siRNAs including but not limited to: TransIT-TKOT™ (Minis, Cat. #MIR 2150), Transmessenger™ (Qiagen, Cat. #301525), Oligofectamine™ and Lipofectamine™ (Invitrogen, Cat. #MIR 12252-011 and Cat. #13778-075), siPORT™ (Ambion, Cat. #1631), DharmaFECT™ (Fisher Scientific, Cat. #T-2001-01). Agents are also commercially available for electroporation-based methods for transfection of siRNA, such as siPORTer™ (Ambion Inc. Cat. #1629). Microinjection techniques can also be used. The small RNA can also be transcribed from an expression construct introduced into the cells, where the expression construct includes a coding sequence for transcribing the small RNA operably linked to one or more transcriptional regulatory sequences. Where desired, plasmids, vectors, or viral vectors can also be used for the delivery of dsRNA or siRNA and such vectors are known in the art. Protocols for each transfection reagent are available from the manufacturer. Additional methods are known in the art and are described, for example in U.S. Patent Application Publication No. 20060058255.

Therapeutics that Prevent or Inhibit Ang-2 Activity

The present invention includes the use of any Ang-2 antagonist compound that prevents or inhibits Ang-2 biological activity (e.g., binding to the Tie-2 receptor, activating RhoA, activating Rho kinase, and upregulating MLC phosphorylation), for the treatment of vascular leak syndromes.

Antibodies

Antibodies that specifically bind to Ang-2, have a high affinity for Ang-2 and/or neutralize or prevent Ang-2 activity and the use of such antibodies in the therapeutic methods are included in the invention. Examples of Ang-2 antibodies include L1-7(N), 2Xcon4, L-10 (N) and AB536 (Oliner et al., Cancer Cell 6:507-516 (2004)), anti-Ang-2 antibodies from Research Diagnostics Inc., (e.g., catalog nos. RDI-ANGIOP2XabR, RDI-ANG218NabG, and RDI-MANGIOP2abrx) and from AbCam Inc. (e.g., catalog nos. Ab18518, Ab8452, and Ab10601). L1-7(N) is an example of an antibody with high affinity for Ang-2. The IC₅₀ for L1-7(N) was 0.071 nM for mouse Ang-2 as compared to >100 nM for Ang-1.

In addition, anti-Ang-1 agonistic antibodies, that function to enhance the activity of Ang-1, for example, by causing Tie-2 phosphorylation or by increasing phosphorylation of the p85 subunit of PI3K, phosphorylation of AKT, activation of Rac1, or activation of p190RhoGAP, are also contemplated by the invention. Antibodies that specifically bind to Tie-2 and selectively inhibit binding of Ang-2 but not Ang-1 to the Tie-2 receptor are also useful in the therapeutic methods of the invention.

Compositions of any of the above antibodies are also included in the invention. Methods for the preparation and use of antibodies for therapeutic purposes are described in several patents including U.S. Pat. Nos. 6,054,297; 5,821,337; 6,365,157; and 6,165,464 and are incorporated herein by reference. Antibodies can be polyclonal or monoclonal; monoclonal antibodies are preferred.

Monoclonal antibodies, particularly those derived from rodents including mice, have been used for the treatment of various diseases; however, there are limitations to their use including the induction of a human anti-mouse immunoglobulin response that causes rapid clearance and a reduction in the efficacy of the treatment. For example, a major limitation in the clinical use of rodent monoclonal antibodies is an anti-globulin response during therapy (Miller et al., Blood, 62:988-995 1983; Schroff et al., Cancer Res., 45:879-885, 1985).

The art has attempted to overcome this problem by constructing “chimeric” antibodies in which an animal antigen-binding variable domain is coupled to a human constant domain (U.S. Pat. No. 4,816,567; Morrison et al., Proc. Natl. Acad. Sci. USA, 81:6851-6855, 1984; Boulianne et al., Nature, 312:643-646, 1984; Neuberger et al., Nature, 314:268-270, 1985). The production and use of such chimeric antibodies are described below.

Anti-Ang-2 antagonistic, anti-Ang-1 agonistic, or anti-Tie-2 antibodies may be produced by methods known in the art. These methods include the immunological method described by Kohler and Milstein (Nature, 256: 495-497, 1975), Kohler and Milstein (Eur. J. Immunol, 6, 511-519, 1976), and Campbell (“Monoclonal Antibody Technology, The Production and Characterization of Rodent and Human Hybridomas” in Burdon et al., Eds., Laboratory Techniques in Biochemistry and Molecular Biology, Volume 13, Elsevier Science Publishers, Amsterdam, 1985), as well as by the recombinant DNA method described by Huse et al. (Science, 246, 1275-1281, 1989), or the cell fusion technique described in Crawford et al., (J. Gen. Virol., 64:697-700, 1983); Kozbor and Roder, (J. Immunol., 4:1275-1280, 1981); and Kozbor et al., (Methods Enzymol., 121:120-140, 1986).

Murine myeloma cell lines useful for the production of monoclonal antibodies can be obtained, for example, from the American Type Culture Collection (ATCC; Manassas, Va.). Human myeloma and mouse-human heteromyeloma cell lines have also been described (Kozbor et al., J. Immunol., 133:3001-3005, 1984; Brodeur et al., Monoclonal Antibody Production Techniques and Applications, Marcel Dekker, Inc., New York, pp. 51-63, 1987).

The antibody may be prepared in any mammal, including mice, rats, rabbits, goats, camels, and humans. The antibody may be a member of one of the following immunoglobulin classes: IgG, IgM, IgA, IgD, or IgE, and the subclasses thereof, and preferably is an IgG antibody. While the preferred animal for producing monoclonal antibodies is mouse, the invention is not so limited; in fact, human antibodies may be used and may prove to be preferable. Such antibodies can be obtained by using human hybridomas (Cole et al., “Monoclonal Antibodies and Cancer Therapy”, Alan R. Liss Inc., p. 77-96, 1985). In the present invention, techniques developed for the production of chimeric antibodies by splicing the genes from a mouse antibody molecule of appropriate antigen specificity together with genes from a human antibody molecule can be used (Morrison et al., Proc. Natl. Acad. Sci. 81, 6851-6855, 1984; Neuberger et al., Nature 312, 604-608, 1984; Takeda et al., Nature 314, 452-454, 1985); such antibodies are within the scope of this invention and are described below.

The invention also includes functional equivalents or derivatives of the antibodies described in this specification. Functional equivalents or derivatives include polypeptides with amino acid sequences substantially identical to the amino acid sequence of the variable or hypervariable regions of the antibodies of the invention. Functional equivalents have binding characteristics comparable to those of the antibodies, and include, for example, chimerized, humanized, fully human, and single chain antibodies or antibody fragments, antibody fragments, and antibodies or antibody fragments fused to a second protein. Methods of producing such functional equivalents are disclosed, for example, in PCT Publication No. WO93/21319; European Patent No. 0 239 400 B1; PCT Publication No. WO89/09622; European Patent Application No. 0338, 745; European Patent Application No. 0332424; and U.S. Pat. No. 4,816,567; Morrison et al., Proc. Natl. Acad. Sci. USA, 81:6851-6855, 1984; Boulianne et al., Nature, 312:643-646, 1984; Neuberger et al., Nature, 314:268-270, 1985, Smith et al., FASEB J. 19:331-341 (2005); and U.S Patent Application Publication Nos. 20050208043 and 20050276802, each of which is herein incorporated by reference.

Chimerized antibodies preferably have constant regions derived substantially or exclusively from human antibody constant regions and variable regions derived substantially or exclusively from the sequence of the variable region from a mammal other than a human. Such humanized antibodies are chimeric immunoglobulin, immunoglobulin chains or fragments thereof (such as Fv, Fab, Fab′, F(ab′)₂ or other antigen-binding subsequences of antibodies) which contain minimal sequence derived from non-human immunoglobulin. Methods for humanizing non-human antibodies are well known in the art (for reviews see Vaswani and Hamilton, Ann. Allergy Asthma Immunol., 81:105-119, 1998 and Carter, Nature Reviews Cancer, 1:118-129, 2001). Generally, a humanized antibody has one or more amino acid residues introduced into it from a source that is non-human. These non-human amino acid residues are often referred to as import residues, which are typically taken from an import variable domain. Humanization can be essentially performed following the methods known in the art (Jones et al., Nature, 321:522-525, 1986; Riechmann et al., Nature, 332:323-329, 1988; and Verhoeyen et al., Science, 239:1534-1536 1988), by substituting rodent CDRs or other CDR sequences for the corresponding sequences of a human antibody. Accordingly, such humanized antibodies are chimeric antibodies wherein substantially less than an intact human variable domain has been substituted by the corresponding sequence from a non-human species (see for example, U.S. Pat. No. 4,816,567). In practice, humanized antibodies are typically human antibodies in which some CDR residues and possibly some FR residues are substituted by residues from analogous sites in rodent antibodies (Presta, Curr. Op. Struct. Biol., 2:593-596, 1992).

Additional methods for the preparation of humanized antibodies can be found in U.S. Pat. Nos. 5,821,337, and 6,054,297, and Carter, (supra) which are all incorporated herein by reference. The humanized antibody is selected from any class of immunoglobulins, including IgM, IgG, IgD, IgA and IgE, and any isotype, including IgG₁, IgG₂, IgG₃, and IgG₄. Where cytotoxic activity is not needed, such as in the present invention, the constant domain is preferably of the IgG₂ class. The humanized antibody may comprise sequences from more than one class or isotype, and selecting particular constant domains to optimize desired effector functions is within the ordinary skill in the art.

Human antibodies can also be produced using various techniques known in the art, including phage display libraries (Marks et al., J. Mol. Biol., 222:581-597, 1991, Winter et al. Annu. Rev. Immunol., 12:433-455, 1994, and Smith et al., supra). The techniques of Cole et al. and Boerner et al. are also useful for the preparation of human monoclonal antibodies (Cole et al., supra; Boerner et al., J. Immunol., 147: 86-95, 1991).

Suitable mammals other than a human include any mammal from which monoclonal antibodies may be made. Examples of mammals other than a human include, for example a rabbit, rat, mouse, horse, goat, or primate; a mouse is preferred.

Functional equivalents of antibodies also include single-chain antibody fragments, also known as single-chain antibodies (scFvs). Single-chain antibody fragments are recombinant polypeptides which typically bind antigens or receptors; these fragments contain at least one fragment of an antibody variable heavy-chain amino acid sequence (V_{H}) tethered to at least one fragment of an antibody variable light-chain sequence (V_{L}) with or without one or more interconnecting linkers. Such a linker may be a short, flexible peptide selected to assure that the proper three-dimensional folding of the V_{L} and V_{H} domains occurs once they are linked so as to maintain the target molecule binding-specificity of the whole antibody from which the single-chain antibody fragment is derived. Generally, the carboxyl terminus of the V_{L} or V_{H} sequence is covalently linked by such a peptide linker to the amino acid terminus of a complementary V_{L} and V_{H} sequence. Single-chain antibody fragments can be generated by molecular cloning, antibody phage display library or similar techniques. These proteins can be produced either in eukaryotic cells or prokaryotic cells, including bacteria.

Single-chain antibody fragments contain amino acid sequences having at least one of the variable regions or CDRs of the whole antibodies described in this specification, but are lacking some or all of the constant domains of those antibodies. These constant domains are not necessary for antigen binding, but constitute a major portion of the structure of whole antibodies. Single-chain antibody fragments may therefore overcome some of the problems associated with the use of antibodies containing part or all of a constant domain. For example, single-chain antibody fragments tend to be free of undesired interactions between biological molecules and the heavy-chain constant region, or other unwanted biological activity. Additionally, single-chain antibody fragments are considerably smaller than whole antibodies and may therefore have greater capillary permeability than whole antibodies, allowing single-chain antibody fragments to localize and bind to target antigen-binding sites more efficiently. Also, antibody fragments can be produced on a relatively large scale in prokaryotic cells, thus facilitating their production. Furthermore, the relatively small size of single-chain antibody fragments makes them less likely than whole antibodies to provoke an immune response in a recipient.

Functional equivalents further include fragments of antibodies that have the same or comparable binding characteristics to those of the whole antibody. Such fragments may contain one or both Fab fragments or the F(ab′)₂ fragment. Preferably the antibody fragments contain all six CDRs of the whole antibody, although fragments containing fewer than all of such regions, such as three, four or five CDRs, are also functional.

Further, the functional equivalents may be or may combine members of any one of the following immunoglobulin classes: IgG, IgM, IgA, IgD, or IgE, and the subclasses thereof.

Equivalents of antibodies are prepared by methods known in the art. For example, fragments of antibodies may be prepared enzymatically from whole antibodies. Preferably, equivalents of antibodies are prepared from DNA encoding such equivalents. DNA encoding fragments of antibodies may be prepared by deleting all but the desired portion of the DNA that encodes the full-length antibody.

DNA encoding chimerized antibodies may be prepared by recombining DNA substantially or exclusively encoding human constant regions and DNA encoding variable regions derived substantially or exclusively from the sequence of the variable region of a mammal other than a human. DNA encoding humanized antibodies may be prepared by recombining DNA encoding constant regions and variable regions other than the CDRs derived substantially or exclusively from the corresponding human antibody regions and DNA encoding CDRs derived substantially or exclusively from a mammal other than a human.

Suitable sources of DNA molecules that encode fragments of antibodies include cells, such as hybridomas, that express the full-length antibody. The fragments may be used by themselves as antibody equivalents, or may be recombined into equivalents, as described above.

The DNA deletions and recombinations described in this section may be carried out by known methods, such as those described in the published patent applications listed above.

Antibodies are isolated and purified using standard art-known methods. For example, antibodies can be screened using standard art-known methods such as ELISA against the Ang-2 peptide antigen or western blot analysis. Non-limiting examples of such techniques are described in Examples II and III of U.S. Pat. No. 6,365,157, herein incorporated by reference.

Purified Proteins

Purified or isolated Ang-1 polypeptides, or fragments thereof, or nucleic acids encoding Ang-1 polypeptides, or fragments thereof, can be used as a therapeutic compound in the methods of the invention. In the setting of tumors, Ang-1 binds to and activates Tie-2 activation of the PI3K/Akt pathway to promote the survival of ECs (Papapetropoulos et al., Lab Invest. 79: 213-223 (1999), Kim et al., Circ. Res. 86: 24-29 (2000)). Ang-1 can also act to upregulate proteins, such as VE-cadherin that stabilize tight inter-endothelial adherens junctions and can activate Rac1 through PI3K and inhibit RhoA through p190RhoGAP. Any fragment of Ang-1 that can bind to Tie-2 or activate Tie-2 signaling (e.g., by receptor phosphorylation, Rac1 activation, p190RhoGAP activation and RhoA inhibition), or both, is included as a preferred fragment of Ang-1 for the therapeutic methods of the invention.

Purified Ang-2 binding proteins that bind to Ang-2 and prevent binding to the Tie-2 receptor can also be used in the methods of the invention. Examples of such Ang-2 binding proteins include soluble fragments of Tie-2 that include the extracellular domain of Tie-2 required to bind to Ang-2 or dominant negative forms of Ang-2.

For any of the purified proteins, or fragment thereof, the proteins are prepared using standard methods known in the art. Analogs or homologs which can bind to or block the biological activity of Ang-2 are also included and can be constructed, for example, by making various substitutions of residues or sequences, deleting terminal or internal residues or sequences not needed for biological activity, or adding terminal or internal residues which may enhance biological activity. Amino acid substitutions, deletions, additions, or mutations can be made to improve expression, stability, or solubility of the protein in the various expression systems. Generally, substitutions are made conservatively and take into consideration the effect on biological activity. Mutations, deletions, or additions in nucleotide sequences constructed for expression of analog proteins or fragments thereof must, of course, preserve the reading frame of the coding sequences and preferably will not create complementary regions that could hybridize to produce secondary mRNA structures such as loops or hairpins which would adversely affect translation of the mRNA.

Therapeutics that Target the Ang-1/Tie-2 Signaling Pathway

The Tie-2 receptor is primarily expressed on endothelial cells, though Tie-2 positive bone marrow derived cells have been described. In the setting of tumors, Ang-1 promotes survival of ECs through Tie-2 activation. Ang-1 activation of Tie-2 leads to receptor phosphorylation and subsequence signal transduction that promotes endothelial cell survival and vessel assembly. As described herein, we have shown that Ang-1 activates Rac1 through PI3K and inhibits RhoA through p190RhoGAP. Ang-2 can bind to Tie-2 but is thought to act as an antagonist to the receptor by blocking receptor phosphorylation. However, the action of Ang-2 on the Tie-2 receptor is context, dose, and duration dependent. We have discovered that Ang-2 can block Tie-2 function under physiologic conditions, resulting in a shift in the balance away from Rac1 activation and towards Rho kinase activity which leads to MLC phosphorylation via either activation of EC MLC kinase or inhibition of myosin phosphatase activity, endothelial cell contraction, and disruption of barrier integrity. Given our identification of the importance of the Tie-2 signaling pathway on EC architecture, any compounds that activate Tie-2 signaling or that block the Ang-2 mediated inactivation of Tie-2 signaling are included as therapeutic compounds of the invention. Such compounds include, for example, compounds that induce Tie-2 biological activity either by increasing levels of Tie-2, binding to and activating Tie-2, or increasing levels or biological activity of downstream effectors of Tie-2 and include, for example, compounds that inhibit or reduce MLC phosphorylation (e.g., compounds that inhibit RhoA GTPase or Rho kinase activity, such as Y27632, compounds that inhibit EC MLC kinase such as ML-7, or compounds that activate myosin phosphatase activity), compounds that activate p190RhoGAP, compounds that activate Rac1, and Tie-2 mutants that are constitutively active.

Modifications of any Ang-2 Antagonist Compounds

The Ang-2 antagonist compounds of the invention (e.g., polypeptide, antibodies, small molecule compounds) can also include any modified forms. Examples of post-translational modifications include but are not limited to phosphorylation, glycosylation, hydroxylation, sulfation, acetylation, isoprenylation, proline isomerization, subunit dimerization or multimerization, and cross-linking or attachment to any other proteins, or fragments thereof, or membrane components, or fragments thereof (e.g., cleavage of the protein from the membrane with a membrane lipid component attached). Modifications that provide additional advantages such as increased affinity, decreased off-rate, solubility, stability and in vivo or in vitro circulating time of the polypeptide, or decreased immunogenicity and include, for example, acetylation, acylation, ADP-ribosylation, amidation, covalent attachment of flavin, covalent attachment of a heme moiety, covalent attachment of a nucleotide or nucleotide derivative, covalent attachment of a lipid or lipid derivative, covalent attachment of phosphotidylinositol, cross-linking, cyclization, disulfide bond formation, demethylation, formation of covalent cross-links, formation of cysteine, formation of pyroglutamate, formylation, gamma-carboxylation, glycosylation, GPI anchor formation, hydroxylation, iodination, methylation, myristoylation, oxidation, pegylation, proteolytic processing, phosphorylation, prenylation, racemization, selenoylation, sulfation, transfer-RNA mediated addition of amino acids to proteins such as arginylation, and ubiquitination. (See, for instance, Creighton, “Proteins: Structures and Molecular Properties,” 2d Ed., W. H. Freeman and Co., N.Y., 1992; “Postranslational Covalent Modification of Proteins,” Johnson, ed., Academic Press, New York, 1983; Seifter et al., Meth. Enzymol., 182:626-646, 1990; Rattan et al., Ann. NY Acad. Sci., 663:48-62, 1992) are also included. The Ang-2 antagonist compound can also include sequence variants of any of the compounds such as variants that include 1, 2, 3, 4, 5, greater than 5, or greater than 10 amino acid alterations such as substitutions, deletions, or insertions with respect to wild type sequence. Additionally, the Ang-2 antagonist compound may contain one or more non-classical amino acids. Non-classical amino acids include, but are not limited to, to the D-isomers of the common amino acids, 2,4-diaminobutyric acid, α-amino isobutyric acid, 4-aminobutyric acid, Abu, 2-amino butyric acid, g-Abu, e-Ahx, 6-amino hexanoic acid, Aib, 2-amino isobutyric acid, 3-amino propionic acid, ornithine, norleucine, norvaline, hydroxyproline, sarcosine, citrulline, homocitrulline, cysteic acid, t-butylglycine, t-butylalanine, phenylglycine, cyclohexylalanine, β-alanine, fluoro-amino acids, designer amino acids such as β-methyl amino acids, Ca-methyl amino acids, Na-methyl amino acids, and amino acid analogs in general. Furthermore, the amino acid can be D (dextrorotary) or L (levorotary).

Additional post-translational modifications encompassed by the invention include, for example, e.g., N-linked or O-linked carbohydrate chains, processing of N-terminal or C-terminal ends), attachment of chemical moieties to the amino acid backbone, chemical modifications of N-linked or O-linked carbohydrate chains, and addition or deletion of an N-terminal methionine residue.

In addition, chemically modified derivatives of the Ang-2 antagonist compounds, which may provide additional advantages such as increased solubility, stability and circulating time of the polypeptide, or decreased immunogenicity (see U.S. Pat. No. 4,179,337) are also included. The chemical moieties for derivitization may be selected from water soluble polymers such as, for example, polyethylene glycol, ethylene glycol/propylene glycol copolymers, carboxymethylcellulose, dextran, polyvinyl alcohol and the like. The Ang-2 antagonist compound may be modified at random positions within the molecule, or at predetermined positions within the molecule and may include one, two, three or more attached chemical moieties.

The polymer may be of any molecular weight, and may be branched or unbranched. For polyethylene glycol, the preferred molecular weight is between about 1 kDa and about 100 kDa (the term “about” indicating that in preparations of polyethylene glycol, some molecules will weigh more, some less, than the stated molecular weight) for ease in handling and manufacturing. Other sizes may be used, depending on the desired therapeutic profile (e.g., the duration of sustained release desired, the effects, if any on biological activity, the ease in handling, the degree or lack of antigenicity and other known effects of the polyethylene glycol to a therapeutic protein or analog). As noted above, the polyethylene glycol may have a branched structure. Branched polyethylene glycols are described, for example, in U.S. Pat. No. 5,643,575; Morpurgo et al., Appl. Biochem. Biotechnol. 56:59-72, (1996); Vorobjev et al., Nucleosides Nucleotides 18:2745-2750, (1999); and Caliceti et al., Bioconjug. Chem. 10:638-646, (1999), the disclosures of each of which are incorporated by reference.

Any of the Ang-2 antagonist compounds of the present invention (e.g., polypeptide, antibodies, or small molecule compounds) may also be modified in a way to form a chimeric molecule comprising Ang-2 antagonist fused to another, heterologous polypeptide or amino acid sequence, such as an Fc sequence, a detectable label, or an additional therapeutic molecule. In one example, an Ang-2 antagonist antibody can be a peptide fused to an Fc fusion protein.

For any of the polypeptides, including antibodies, that are used in the methods of the invention, the nucleic acids encoding the polypeptides or antibodies, or fragments thereof, are also useful in the methods of the invention using standard techniques for gene therapy known in the art and described herein. The invention also includes Ang-2 antagonist compounds, such as mimetics, based on modeling the 3-dimensional structure of a polypeptide or peptide fragment and using rational drug design to provide potential inhibitor compounds with particular molecular shape, size and charge characteristics. Following identification of an Ang-2 antagonist compound, suitable modeling techniques known in the art can be used to study the functional interactions and design mimetic compounds which contain functional groups arranged in such a manner that they could reproduced those interactions. The designing of mimetics to a known pharmaceutically active compound is a known approach to the development of pharmaceuticals based on a lead compound. This might be desirable where the active compound is difficult or expensive to synthesize or where it is unsuitable for a particular method of administration, e.g. peptides are not well suited as active agents for oral compositions as they tend to be quickly degraded by proteases in the alimentary canal. Mimetic design, synthesis and testing may be used to avoid randomly screening large number of molecules for a target property. The mimetic or mimetics can then be screened to see whether they reduce or inhibit Ang-2 biological activity and further optimization or modification can then be carried out to arrive at one or more final mimetics for in vivo or clinical testing.

### IL-2 and Rituximab Therapy Applications

High dose interleuin-2 (HD IL-2) is the only FDA-approved therapy for metastatic renal cell cancer and is also used as salvage therapy in patients with metastatic melanoma. HD IL-2 is believed to activate a patient's own T lymphocytes and NK cells to attack existing tumor. Though the response rate is approximately 10%, those who do improve have durable response measurable in years.

Up to 65% of subjects receiving HD IL-2 develop a dose-limiting vascular leak syndrome characterized by marked hypermutability leading to diffuse extravasation of fluid, particularly in the lung, where it can provoke respiratory distress. We have shown that serum Ang-2 is elevated following HD IL-2 therapy. In particular, we have also shown that serial measurements of Ang-2 in a patient undergoing HD IL-2 therapy show a rise in Ang-2 levels for each day of infusion followed by a rapid decline over 24 hours. These results demonstrate the role of Ang-2 in vascular leak syndrome that occurs in patients undergoing HD IL-2 therapy. We have also shown that culturing human pulmonary microvascular endothelial cells (HMVEC-L) in serum from a patient with high Ang-2 causes actin stress fiber formation and endothelial gap formation. Accordingly, the invention includes the use of Ang-2 antagonists to treat, prevent, or reduce vascular leak syndrome, or the risk of developing vascular leak syndrome in patients undergoing HD IL-2 therapy. Ang-2 antagonists can be administered at anytime during the course of HD IL-2 therapy or prior to HD IL-2 therapy to prevent vascular leak syndrome from occurring. In one example, an Ang-2 antibody, such as L1-7(N), or functional derivatives of fragments thereof, is administered to a patient undergoing HD IL-2 therapy. Desirably, the patient's Ang-2 levels are monitored during therapy and the anti-Ang-2 antibody is administered to reduce Ang-2 levels or to maintain Ang-2 levels to a level that is considered within the normal range (e.g., a normal reference level of Ang-2 is less than 5 ng/ml serum, preferably less than 4 ng/ml, 3 ng/ml, 2 ng/ml, or less than 1 ng/ml serum). The anti-Ang-2 antibody can also be administered after HD IL-2 therapy is complete, to prevent against vascular leak syndrome development post-IL-2 therapy.

Rituximab (Rituxan™) is a chimeric monoclonal antibody directed against CD20 that has been used for the treatment of hematological cancers including non-Hodgkin's lymphoma, lymphoid leukemia, and highly aggressive lymphomas. Vascular leak is a toxic side effect that is sometimes associated with rituximab therapy. The invention also includes the use of Ang-2 antagonists to treat, prevent, or reduce vascular leak syndrome, or the risk of developing vascular leak syndrome in patients undergoing rituximab therapy. Ang-2 antagonists can be administered at anytime during the course of rituximab therapy or prior to rituximab therapy to prevent vascular leak syndrome from occurring. In one example, an Ang-2 antibody, such as L1-7(N), or functional derivatives or fragments thereof, is administered to a patient undergoing rituximab therapy. Desirably, the patient's Ang-2 levels are monitored during therapy and the anti-Ang-2 antibody is administered to reduce Ang-2 levels or to maintain Ang-2 levels to a level that is considered within the normal range. The anti-Ang-2 antibody can also be administered after rituximab therapy is complete, to prevent against vascular leak syndrome development post-IL-2 therapy.

### Combination Therapies for Vascular Leak Disorders

In various embodiments Ang-2 antagonists can be provided in conjunction (e.g., before, during, or after) with additional vascular leak therapies to prevent or reduce a vascular leak disorder, including sepsis, ARDS, and ALI. Treatment therapies that can be used in combination with the methods of the invention include but are not limited to antibiotics, surgical drainage of infected fluid collections, fluid replacement, and appropriate support for organ dysfunction, including, for example, hemodialysis in kidney failure, mechanical ventilation in pulmonary dysfunction, transfusion of blood plasma, platelets, and coagulation factors to stabilize blood coagulation, and drug and fluid therapy for circulatory failure. Additional therapies can include activated protein C therapy (drotrecogin) and corticosteroid treatment, vasopressin, inhibitors of MLC kinase, inhibitors of VEGF (e.g., avastin), inhibitors of PlGF, inhibitors of NFkB (e.g., panepoxydone), inhibitors of TNF-α, inhibitors of IL-1, IL-6, and inhibitors of TGF-β. Desirably, Ang-2 antagonist compounds can be formulated alone or in combination with any additional vascular leak therapies, either described herein or known in the art. A combination of any two or more of the Ang-2 antagonist compounds described herein can also be used for the treatment of vascular leak. In one example, an Ang-2 antagonist compound that specifically blocks Ang-2 activity (e.g., an Ang-2 antibody) is combined with a compound that is an antagonist of Ang-2 or Tie-2 (e.g., an isolated Ang-1 fragment that binds Tie-2 and prevents Ang-2 from binding to Tie-2 or that shifts the cellular balance towards p190RhoGAP activation and away from RhoA activation.

### Therapeutic Formulations

The dosage and the timing of administering the Ang-2 antagonist compound of the invention depends on various clinical factors including the overall health of the subject and the severity of the symptoms of the vascular leak. The invention includes the use of Ang-2 antagonists to treat, prevent or reduce vascular leak disorders, or the risk of developing vascular leak disorders in a subject. The Ang-2 antagonist can be administered at anytime, for example, after diagnosis or detection of a vascular leak or a condition associated with vascular leak (e.g., using the diagnostic methods known in the art or described herein), or for prevention of a vascular leak disorder in subjects that have not yet been diagnosed with a vascular leak disorder but are at risk of developing such a disorder (e.g., subjects suffering from or being treated for sepsis), after a risk of developing a vascular leak disorder is determined.

The Ang-2 antagonist compounds of the present invention can be formulated and administered in a variety of ways, e.g., those routes known for specific indications, including, but not limited to, topically, orally, subcutaneously, bronchial injection, intravenously, intracerebrally, intranasally, transdermally, intraperitoneally, intramuscularly, intrapulmonary, vaginally, rectally, intraarterially, intralesionally, parenterally, intraventricularly in the brain, or intraocularly. For example, the Ang-2 antagonist compound can be in the form of a pill, tablet, capsule, liquid, or sustained release tablet for oral administration; or a liquid for intravenous, subcutaneous or administration; a polymer or other sustained release vehicle for local administration; an ointment, cream, gel, liquid, or patch for topical administration.

For example, continuous systemic infusion or periodic injection of the Ang-2 antagonist compound can be used to treat or prevent the disorder. Treatment can be continued for a period of time ranging from 1 day through the lifetime of the subject, more preferably 1 to 100 days, and most preferably 1 to 20 days and most preferably, until the symptoms of vascular leak are reduced or removed. Dosages vary depending on the compound and the severity of the condition. The Ang-2 antagonist compounds can be administered continuously by infusion, using a constant- or programmable-flow implantable pump, or by periodic injections. Sustained release systems can also be used. Semipermeable, implantable membrane devices are also useful as a means for delivering Ang-2 antagonists in certain circumstances. In another embodiment, the Ang-2 antagonist compound is administered locally, e.g., by inhalation, and can be repeated periodically.

Therapeutic formulations are prepared using standard methods known in the art by mixing the active ingredient having the desired degree of purity with optional physiologically acceptable carriers, excipients or stabilizers (Remington's Pharmaceutical Sciences (20^{th} edition), ed. A. Gennaro, 2000, Lippincott, Williams & Wilkins, Philadelphia, Pa.), in the form of lyophilized formulations or aqueous solutions. Acceptable carriers, include saline, or buffers such as phosphate, citrate and other organic acids; antioxidants including ascorbic acid; low molecular weight (less than about 10 residues) polypeptides; proteins, such as serum albumin, gelatin or immunoglobulins; hydrophilic polymers such as polyvinylpyrrolidone, amino acids such as glycine, glutamine, asparagines, arginine or lysine; monosaccharides, disaccharides, and other carbohydrates including glucose, mannose, or dextrins; chelating agents such as EDTA; sugar alcohols such as mannitol or sorbitol; salt-forming counterions such as sodium; and/or nonionic surfactants such as TWEEN™, PLURONICS™, or PEG.

Optionally, but preferably, the formulation contains a pharmaceutically acceptable salt, preferably sodium chloride, and preferably at about physiological concentrations. Optionally, the formulations of the invention can contain a pharmaceutically acceptable preservative. In some embodiments the preservative concentration ranges from 0.1 to 2.0%, typically v/v. Suitable preservatives include those known in the pharmaceutical arts. Benzyl alcohol, phenol, m-cresol, methylparaben, and propylparaben are preferred preservatives. Optionally, the formulations of the invention can include a pharmaceutically acceptable surfactant. Preferred surfactants are non-ionic detergents. Preferred surfactants include Tween 20 and pluronic acid (F68). Suitable surfactant concentrations are 0.005 to 0.02%.

The dosage of the Ang-2 antagonistic compound will depend on other clinical factors such as weight and condition of the subject and the route of administration of the compound. For treating subjects, between approximately 0.1 mg/kg to 500 mg/kg body weight of the Ang-2 antagonistic compound can be administered. A more preferable range is 1 mg/kg to 50 mg/kg body weight with the most preferable range being from 1 mg/kg to 25 mg/kg body weight. Depending upon the half-life of the Ang-2 antagonistic compound in the particular subject, the Ang-2 antagonistic compound can be administered between several times per day to once a week. The methods of the present invention provide for single as well as multiple administrations, given either simultaneously or over an extended period of time.

If antibodies are used in vivo for the treatment or prevention of vascular leak, the antibodies of the subject invention are administered to the subject in therapeutically effective amounts. Preferably, the antibodies are administered parenterally or intravenously by continuous infusion. The dose and dosage regimen depends upon the severity of the disease, and the overall health of the subject. The amount of antibody administered is typically in the range of about 0.001 to about 10 mg/kg of subject weight, preferably 0.01 to about 5 mg/kg of subject weight.

For parenteral administration, the antibodies are formulated in a unit dosage injectable form (solution, suspension, emulsion) in association with a pharmaceutically acceptable parenteral vehicle. Such vehicles are inherently nontoxic, and non-therapeutic. Examples of such vehicles are water, saline, Ringer's solution, dextrose solution, and 5% human serum albumin. Nonaqueous vehicles such as fixed oils and ethyl oleate may also be used. Liposomes may be used as carriers. The vehicle may contain minor amounts of additives such as substances that enhance isotonicity and chemical stability, e.g., buffers and preservatives. The antibodies typically are formulated in such vehicles at concentrations of about 1 mg/ml to 10 mg/ml.

The dosage required depends on the choice of the route of administration; the nature of the formulation; the nature of the subject's illness; the subject's size, weight, surface area, age, and sex; other drugs being administered; and the judgment of the attending physician. Wide variations in the needed dosage are to be expected in view of the variety of polypeptides and fragments available and the differing efficiencies of various routes of administration. For example, oral administration would be expected to require higher dosages than administration by intravenous injection. Variations in these dosage levels can be adjusted using standard empirical routines for optimization as is well understood in the art. Administrations can be single or multiple (e.g., 2-, 3-, 6-, 8-, 10-, 20-, 50-, 100-, 150-, or more). Encapsulation of the polypeptide in a suitable delivery vehicle (e.g., polymeric microparticles or implantable devices) may increase the efficiency of delivery, particularly for oral delivery.

Alternatively, a polynucleotide containing a nucleic acid sequence encoding an Ang-2 antagonist can be delivered to the appropriate cells in the subject. Expression of the coding sequence can be directed to any cell in the body of the subject. In certain embodiments, expression of the coding sequence can be directed to the lung. This can be achieved by, for example, the use of polymeric, biodegradable microparticle or microcapsule delivery devices known in the art.

The nucleic acid can be introduced into the cells by any means appropriate for the vector employed. Many such methods are well known in the art (Sambrook et al., supra, and Watson et al., Recombinant DNA, Chapter 12, 2d edition, Scientific American Books, 1992). Examples of methods of gene delivery include liposome mediated transfection, electroporation, calcium phosphate/DEAE dextran methods, gene gun, and microinjection.

In gene therapy applications, genes are introduced into cells in order to achieve in vivo synthesis of a therapeutically effective genetic product. “Gene therapy” includes both conventional gene therapy where a lasting effect is achieved by a single treatment, and the administration of gene therapeutic agents, which involves the one time or repeated administration of a therapeutically effective DNA or mRNA. Standard gene therapy methods typically allow for transient protein expression at the target site ranging from several hours to several weeks. Re-application of the nucleic acid can be utilized as needed to provide additional periods of expression of Ang-2 antagonist compounds.

Another way to achieve uptake of the nucleic acid is using liposomes, prepared by standard methods. The vectors can be incorporated alone into these delivery vehicles or co-incorporated with tissue-specific antibodies. Alternatively, one can prepare a molecular conjugate composed of a plasmid or other vector attached to poly-L-lysine by electrostatic or covalent forces. Poly-L-lysine binds to a ligand that can bind to a receptor on target cells (Cristiano et al. J. Mol. Med. 73:479, 1995). Alternatively, tissue specific targeting can be achieved by the use of tissue-specific transcriptional regulatory elements which are known in the art. Delivery of “naked DNA” (i.e., without a delivery vehicle) to an intramuscular, intradermal, or subcutaneous site is another means to achieve in vivo expression.

Gene delivery using viral vectors such as adenoviral, retroviral, lentiviral, or adeno-associated viral vectors can also be used. Numerous vectors useful for this purpose are generally known and have been described (Miller, Human Gene Therapy 15:14, 1990; Friedman, Science 244:1275-1281, 1989; Eglitis and Anderson, BioTechniques 6:608-614, 1988; Tolstoshev and Anderson, Current Opinion in Biotechnology 1:55-61, 1990; Sharp, The Lancet 337:1277-1278, 1991; Cornetta et al., Nucleic Acid Research and Molecular Biology 36:311-322, 1987; Anderson, Science 226:401-409, 1984; Moen, Blood Cells 17:407-416, 1991; Miller and Rosman, Biotechniques 7:980-990, 1989; Rosenberg et al., N. Engl. J. Med 323:370, 1990, Groves et al., Nature, 362:453-457, 1993; Horrelou et al., Neuron, 5:393-402, 1990; Jiao et al., Nature 362:450-453, 1993; Davidson et al., Nature Genetics 3:2219-2223, 1993; Rubinson et al., Nature Genetics 33, 401-406, 2003; U.S. Pat. Nos. 6,180,613; 6,410,010; 5,399,346 all hereby incorporated by reference). These vectors include adenoviral vectors and adeno-associated virus-derived vectors, retroviral vectors (e.g., Moloney Murine Leukemia virus based vectors, Spleen Necrosis Virus based vectors, Friend Murine Leukemia based vectors, lentivirus based vectors (Lois C. et al., Science, 295:868-872, 2002; Rubinson et al., supra), papova virus based vectors (e.g., SV40 viral vectors), Herpes-Virus based vectors, viral vectors that contain or display the Vesicular Stomatitis Virus G-glycoprotein Spike, Semliki-Forest virus based vectors, Hepadnavirus based vectors, and Baculovirus based vectors.

In the relevant polynucleotides (e.g., expression vectors), the nucleic acid sequence encoding the Ang-2 antagonistic polypeptide (including an initiator methionine and optionally a targeting sequence) is operatively linked to a promoter or enhancer-promoter combination. Short amino acid sequences can act as signals to direct proteins to specific intracellular compartments. Such signal sequences are described in detail in U.S. Pat. No. 5,827,516, incorporated herein by reference in its entirety.

An ex vivo strategy can also be used for therapeutic applications. Ex vivo strategies involve transfecting or transducing cells obtained from the subject with a polynucleotide encoding an Ang-2 antagonistic polypeptide. The transfected or transduced cells are then returned to the subject. Such cells act as a source of the Ang-2 antagonistic polypeptide for as long as they survive in the subject.

The ex vivo methods include the steps of harvesting cells from a subject, culturing the cells, transducing them with an expression vector, and maintaining the cells under conditions suitable for expression of the Ang-2 antagonistic polypeptide or functional fragment. These methods are known in the art of molecular biology. The transduction step is accomplished by any standard means used for ex vivo gene therapy including calcium phosphate, lipofection, electroporation, viral infection, and biolistic gene transfer. Alternatively, liposomes or polymeric microparticles can be used. Cells that have been successfully transduced can then be selected, for example, for expression of the coding sequence or of a drug resistance gene. The cells may then be lethally irradiated (if desired) and injected or implanted into the patient. For example, Ang-2 antagonist therapy for the treatment or prevention of vascular leak associated with HD IL-2 therapy can be implemented by bringing in a future recipient of HD IL-2 weeks before HD IL-2 administration to harvest cells that can be treated ex vivo, then reintroduced around the time HD IL-2 is given.

Where sustained release administration of Ang-2 antagonist is desired in a formulation with release characteristics suitable for the treatment of any disease or disorder requiring administration of the Ang-2 antagonist, microencapsulation of the Ang-2 antagonist is contemplated. Micro encapsulation of recombinant proteins for sustained release has been successfully performed with human growth hormone (rhGH), interferon-(rhIFN-), interleukin-2, and MN rgp120. Johnson et al., Nat. Med., 2:795-799, 1996; Yasuda, Biomed. Ther., 27:1221-1223, 1993; Hora et al., Bio/Technology, 8:755-758 1990; Cleland, “Design and Production of Single Immunization Vaccines Using Polylactide Polyglycolide Microsphere Systems,” in “Vaccine Design: The Subunit and Adjuvant Approach,” Powell and Newman, eds., Plenum Press: New York, pp. 439-462, 1995; WO 97/03692; WO 96/40072; WO 96/07399; and U.S. Pat. No. 5,654,010.

The sustained-release formulations may include those developed using ply-lactic-coglycolic acid (PLGA) polymer. The degradation products of PLGA, lactic and glycolic acids, can be cleared quickly within the human body. Moreover, the degradability of this polymer can be adjusted from months to years depending on its molecular weight and composition. See Lewis, “Controlled release of bioactive agents from lactide/glycolide polymer,” in M. Chasin and Dr. Langer (Eds.), Biodegradable Polymers as Drug Delivery Systems (Marcel Dekker: New York, pp. 1-41, 1990.

The Ang-2 antagonist for use in the present invention may also be modified in a way to form a chimeric molecule comprising Ang-2 antagonist fused to another, heterologous polypeptide or amino acid sequence, such as an Fc sequence or an additional therapeutic molecule (e.g., a chemotherapeutic or cytotoxic agent).

The Ang-2 antagonist compound can be packaged alone or in combination with other therapeutic compounds as a kit. Non-limiting examples include kits that contain, e.g., two pills, a pill, and a powder, a suppository and a liquid in a vial, two topical creams, etc.

The kit can include optional components that aid in the administration of the unit dose to patients, such as vials for reconstituting powder forms, syringes for injection, customized IV delivery systems, inhalers, etc. Additionally, the unit dose kit can contain instructions for preparation and administration of the compositions. The kit may be manufactured as a single use unit dose for one patient, multiple uses for a particular patient (at a constant dose or in which the individual compounds may vary in potency as therapy progresses); or the kit may contain multiple doses suitable for administration to multiple patients (“bulk packaging”). The kit components may be assembled in cartons, blister packs, bottles, tubes, and the like.

### Ang-2 Agonists for the Induction of Vascular Leak

For certain applications, a temporary state of vascular leak is desired. Such applications include the need to break down the blood-brain barrier to treat diseases such as brain diseases or brain tumors, in which CNS penetration is needed. Other therapeutic applications of vascular leak include localized breakdown of the capillary permeability barrier to promote fluid and phagocyte extravasation (to clear infection from poorly perfused areas such as synovial cavities), and to promote loss of proteins and other molecules into urine by increasing renal capillary permeability. For such applications, any compound that shifts the GTPase balance away in favor of RhoA activity over Rac1 can be used. For example, an Ang-2 agonist can be used to induce the state of vascular leak. Examples of Ang-2 agonist compounds that can be used include a purified Ang-2 protein, an isolated nucleic acid molecule encoding an Ang-2 polypeptide; an agonistic anti-Ang-2 antibody; a compound that binds to Tie-2 and blocks Ang-1 binding but not Ang-2 binding; a compound that induces MLC phosphorylation; a compound that activates Rho kinase activity; a compound that inhibits Rac1 or p190RhoGAP biological activity or expression (e.g., siRNA, antisense nucleobase oligomers, or antibodies that specifically bind Rac1 or p190RhoGAP); a compound that inhibits Ang-1 biological activity or expression (e.g., siRNA, antisense nucleobase oligomers, or antibodies that specifically bind Ang-1); and a compound that induces RhoA biological activity or expression levels (e.g., a purified RhoA protein).

Any of the Ang-2 agonistic compounds can be prepared and administered using any of the methods described for the Ang-2 antagonist compounds.

### Diagnostics

We have shown that Ang-2 levels are elevated in patients with vascular leak syndrome and impairment in gas exchange and that measurement of Ang-2 levels can be used as a tool to diagnose or predict the prognosis of a subject having or at risk for sepsis or any other disorders characterized by vascular leak, hypotension, or a procoagulant state. We have also shown that Ang-2 levels are elevated following HD IL-2 therapy. In particular, we have also shown that serial measurements of Ang-2 in a patient undergoing HD IL-2 therapy showing a rise in Ang-2 levels for each day of infusion followed by a rapid decline over 24 hours.

The present invention features methods and compositions to predict, diagnose, and stratify patients at risk for developing vascular leak or hypotension using Ang-2 nucleic acid molecules and polypeptides. The methods and compositions can include the measurement of Ang-2 polypeptides, either free or bound to another molecule, or any fragments or derivatives thereof. The methods can include measurement of absolute levels of Ang-2 or relative levels as compared to a normal reference. For example, a serum level of Ang-2 that is less than 5 ng/ml, 4 ng/ml, 3 ng/ml, 2 ng/ml, or less than 1 ng/ml serum is considered to be predictive of a low risk of vascular leak or of a good outcome in a patient diagnosed with a vascular leak syndrome. A serum level of Ang-2 that is greater than 5 ng/ml, 10 ng/ml serum or most preferably greater than 20 ng/ml is considered diagnostic of vascular leak or of a poor outcome in a subject already diagnosed with a vascular leak syndrome.

For diagnoses based on relative levels of Ang-2, a subject having a vascular leak disorder or hypotension, or a propensity to develop such a condition will show an alteration (e.g., an increase of 10%, 20%, 30%, 40%, 50%, 60%, 70%, 80%, 90%, or more), in the expression of an Ang-2 polypeptide as compared to a normal reference sample or level. A normal reference sample can be, for example, a prior sample taken from the same subject prior to the development of the vascular leak syndrome or of symptoms suggestive of vascular leak syndrome, a sample from a subject not having any vascular leak syndrome or hypotension or a sample of a purified reference polypeptide at a known normal concentration (i.e., not indicative of a vascular leak syndrome or hypotension). By “reference standard or level” is meant a value or number derived from a reference sample. A normal reference standard or level can be a value or number derived from a normal subject.

For diagnostic assays that include measuring Ang-2 polypeptide, the Ang-2 polypeptide can include full-length Ang-2 polypeptide, degradation products, alternatively spliced isoforms of Ang-2 polypeptide, enzymatic cleavage products of Ang-2 polypeptide, and the like. In one example, an antibody that specifically binds Ang-2 polypeptide is used for the diagnosis of a vascular leak or hypotension, or to identify a subject at risk of developing such conditions, or to provide a prognosis for a subject already suffering from such a condition.

Standard methods may be used to measure levels of Ang-2 polypeptide in any bodily fluid, including, but not limited to, urine, blood, serum, plasma, saliva, amniotic fluid, or cerebrospinal fluid. Such methods include immunoassay, ELISA, Western blotting using antibodies that specifically bind to Ang-2 polypeptide, and quantitative enzyme immunoassay techniques. ELISA assays are the preferred method for measuring levels of Ang-2 polypeptide. Increases in the levels of Ang-2 polypeptide, as compared to normal controls, are considered a positive indicator of a vascular leak syndrome, or the propensity to develop such a syndrome, or a poor prognosis in a subject already suffering from such a condition.

Ang-2 nucleic acid molecules, or substantially identical fragments thereof, or fragments or oligonucleotides of Ang-2 that hybridize to Ang-2 at high stringency may be used as a probe to monitor expression of Ang-2 nucleic acid molecules in the diagnostic methods of the invention. Increases in the levels of Ang-2 nucleic acid molecules, as compared to normal controls, are considered a positive indicator of a vascular leak syndrome, hypotension, or the propensity to develop such a syndrome, or a poor prognosis in a subject already suffering from such a condition. Any of the Ang-2 nucleic acid molecules above can also be used to identify subjects having a genetic variation, mutation, or polymorphism in a Ang-2 nucleic acid molecule that are indicative of a predisposition to develop the conditions. These polymorphisms may affect Ang-2 nucleic acid or polypeptide expression levels or biological activity. Detection of genetic variation, mutation, or polymorphism relative to a normal, reference sample can be used as a diagnostic indicator of a vascular leak, vascular leak syndrome, hypotension, or the propensity to develop such a condition.

Such genetic alterations may be present in the promoter sequence, an open reading frame, intronic sequence, or untranslated 3′ region of a Ang-2 gene. Information related to genetic alterations can be used to diagnose a subject as having a vascular leak syndrome, hypotension, or the propensity to develop such a condition. As noted throughout, specific alterations in the levels of biological activity of Ang-2 can be correlated with the likelihood of a vascular leak syndrome, hypotension, or the propensity to develop such a condition. As a result, one skilled in the art, having detected a given mutation, can then assay one or more of the biological activities of the protein to determine if the mutation causes or increases the likelihood of a vascular leak syndrome, hypotension, or the propensity to develop such a condition.

In one embodiment, a subject having a vascular leak disorder, hypotension, or the propensity to develop such a disorder, will show an increase in the expression of a nucleic acid encoding Ang-2 or an alteration in Ang-2 polypeptide levels. Methods for detecting such alterations are standard in the art and are described in Ausubel et al., supra. In one example Northern blotting or PCR (e.g., RT-PCR or real-time) is used to detect Ang-2 mRNA levels.

In another embodiment, hybridization at high stringency with PCR probes that are capable of detecting an Ang-2 nucleic acid molecule, including genomic sequences, or closely related molecules, may be used to hybridize to a nucleic acid sequence derived from a subject having vascular leak disorder, hypotension, or the propensity to develop such a disorder. The specificity of the probe, whether it is made from a highly specific region, e.g., the 5′ regulatory region, or from a less specific region, e.g., a conserved motif, and the stringency of the hybridization or amplification (maximal, high, intermediate, or low), determine whether the probe hybridizes to a naturally occurring sequence, allelic variants, or other related sequences. Hybridization techniques may be used to identify mutations indicative of vascular leak syndrome, hypotension, or the propensity to develop such a condition in an Ang-2 nucleic acid molecule, or may be used to monitor expression levels of a gene encoding an Ang-2 polypeptide (for example, by Northern analysis, Ausubel et al., supra).

In one embodiment, the level of Ang-2 polypeptide or nucleic acid, or any combination thereof, is measured at least two different times and an alteration in the levels (e.g., by 10%, 20%, 30%, 40%, 50%, 60%, 70%, 80%, 90%, or more) over time is used as an indicator of a vascular leak disorder, hypotension, or the propensity to develop such a disorder. For example, once a subject is diagnosed with sepsis, serum samples can be taken at regular intervals (e.g., every 2 hours, 4 hours, 6 hours, 8 hours, 12 hours, 24 hours, every two days, or less frequently) to determine the level of Ang-2 polypeptide or nucleic acid. If the level of Ang-2 increases over the serial measurements, this is considered a diagnostic indicator of vascular leak disorder, hypotension, or the propensity to develop such a disorder, or, if the subject is already determined to have such a condition, this is considered an indicator of a poor prognosis. In one example, serial samples can be taken from a subject being treated with HD IL-2 therapy or having sepsis and if the level of Ang-2 increases over time, the subject is diagnosed with and/or treated for (either prophylactically or therapeutically) vascular leak using the therapeutic methods of the invention or those known in the art.

The diagnostic methods described herein can be used individually or in combination with any other diagnostic method described herein for a more accurate diagnosis of the presence of, severity of, or estimated time of vascular leak disorder hypotension, or the propensity to develop such a disorder. In additional preferred embodiments, other known diagnostic methods for vascular leak disorder, hypotension, or the propensity to develop such a disorder, can be used in combination with the methods described herein. Examples include the use of markers such as TNF-α, IL-1, IL-6, VEGF, and PlGF for the diagnosis of vascular leak disorder, hypotension, or the propensity to develop such a disorder. It should be noted that such markers are known to be elevated early in the course of vascular leak disorders such as sepsis but may be associated with temporal variation in the levels during the course of the disorder. For each of these markers, the level can be compared to a level or sample from a known normal reference.

### Diagnostic Kits

The invention also provides for a diagnostic test kit. For example, a diagnostic test kit can include antibodies that specifically bind to Ang-2 polypeptide, and components for detecting, and more preferably evaluating binding between the antibodies and the Ang-2 polypeptide. For detection, either the antibody or the Ang-2 polypeptide is labeled, and either the antibody or the Ang-2 polypeptide is substrate-bound, such that the Ang-2 polypeptide-antibody interaction can be established by determining the amount of label attached to the substrate following binding between the antibody and the Ang-2 polypeptide. An ELISA is a common, art-known method for detecting antibody-substrate interaction and can be provided with the kit of the invention. Ang-2 polypeptides can be detected in virtually any bodily fluid, such as urine, plasma, blood serum, semen, or cerebrospinal fluid. A kit that determines an alteration in the level of Ang-2 polypeptide relative to a reference, such as the level present in a normal control, is useful as a diagnostic kit in the methods of the invention. The kit can also contain a standard curve indicating levels of Ang-2 that fall within the normal range and levels that would be considered diagnostic of vascular leak disorder, hypotension, or the propensity to develop such a disorder. Desirably, the kit will contain instructions for the use of the kit. In one example, the kit contains instructions for the use of the kit for the diagnosis of a vascular leak disorder, hypotension, or the propensity to develop such a disorder. In yet another example, the kit contains instructions for the use of the kit to monitor therapeutic treatment or dosage regimens.

### Subject Monitoring

The diagnostic methods described herein can also be used to monitor vascular leak syndromes during therapy or to determine the dosages of therapeutic compounds. In one embodiment, the levels of Ang-2 polypeptide are measured repeatedly as a method of not only diagnosing vascular leak disorders but also monitoring the treatment, prevention, or management of the disease. In order to monitor the progression of a vascular leak disorder in a subject, subject samples can be obtained at several points and compared. For example, the diagnostic methods can be used to monitor subjects during HD IL-2 therapy. In this example, serum samples can be obtained before treatment with HD IL-2, again during treatment with HD IL-2, and again after treatment with HD IL-2. In this example, the patient's Ang-2 levels are closely monitored and if they begin to increase during therapy, the patient can be treated for vascular leak or HD IL-2 therapy can be modified, reduced, or stopped completely, as determined by the clinician. In another example, serum samples can be obtained from a subject undergoing therapy for severe sepsis and Ang-2 levels can be monitored as an indicator of the efficacy of the therapy. The therapeutic regimen can then be modified to maintain or reduce the levels of Ang-2 to within the normal range. The monitoring methods of the invention can also be used, for example, in assessing the efficacy of a particular drug in a subject, determining dosages, or in assessing vascular leak progression or status.

### Screening Assays

As discussed above, we have discovered that Ang-2 can provoke pathologic structural changes in endothelium that lead to changes in barrier function and, ultimately, in increased vascular permeability. Based on these discoveries, compositions of the invention are useful for the high-throughput low-cost screening of candidate compounds to identify those that modulate, preferably decrease (e.g., by at least 10%, 20%, 30%, 40%, 50%, 60%, 70%, 80%, 90%, 95%, or more), the expression or biological activity of Ang-2 for the treatment of vascular leak disorders or a propensity to develop a vascular leak disorder.

Any number of methods are available for carrying out screening assays to identify new candidate compounds that modulate, preferably increase, the expression of an Ang-2 nucleic acid molecule. In one working example, candidate compounds are added at varying concentrations to the culture medium of cultured cells expressing an Ang-2 nucleic acid sequence. Gene expression is then measured, for example, by microarray analysis, Northern blot analysis (Ausubel et al., Current Protocols in Molecular Biology, Wiley Interscience, New York, 2001), or RT-PCR, using any appropriate fragment prepared from the nucleic acid molecule as a hybridization probe. The level of gene expression in the presence of the candidate compound is compared to the level measured in a control culture medium lacking the candidate compound. A compound that promotes an alteration such as a decrease in the expression of an Ang-2 gene, nucleic acid molecule, or polypeptide, or a functional equivalent thereof, is considered useful in the invention; such a molecule may be used, for example, as a therapeutic to delay, ameliorate, or treat a vascular leak disorder or hypotension in a subject.

In another working example, an Ang-2 nucleic acid is expressed as a transcriptional or translational fusion with a detectable reporter, and expressed in an isolated cell (e.g., mammalian or insect cell) under the control of a heterologous promoter, such as an inducible promoter. The cell expressing the fusion protein is then contacted with a candidate compound, and the expression of the detectable reporter in that cell is compared to the expression of the detectable reporter in an untreated control cell. A candidate compound that decreases the expression of an Ang-2 detectable reporter fusion is a compound that is useful as a therapeutic to delay, ameliorate, or treat a vascular leak disorder or hypotension in a subject. In preferred embodiments, the candidate compound alters the expression of a reporter gene fused to a nucleic acid or nucleic acid.

In another working example, the effect of candidate compounds may be measured at the level of polypeptide expression using the same general approach and standard immunological techniques, such as Western blotting or immunoprecipitation with an antibody specific for an Ang-2 polypeptide. For example, immunoassays may be used to detect or monitor the expression of at least one of the polypeptides of the invention in an organism. Polyclonal or monoclonal antibodies that are capable of binding to such a polypeptide may be used in any standard immunoassay format (e.g., ELISA, Western blot, or RIA assay) to measure the level of the polypeptide. In some embodiments, a compound that promotes an alteration, such as a decrease, in the expression or biological activity of an Ang-2 polypeptide is considered particularly useful. Again, such a molecule may be used, for example, as a therapeutic to delay, ameliorate, or treat a vascular leak disorder or hypotension in a subject.

In yet another working example, candidate compounds may be screened for those that specifically bind to an Ang-2 polypeptide or an Ang-2 receptor such as Tie-2. The efficacy of such a candidate compound is dependent upon its ability to interact with such a polypeptide or a functional equivalent thereof. Such an interaction can be readily assayed using any number of standard binding techniques and functional assays (e.g., those described in Ausubel et al., supra). In one embodiment, a candidate compound may be tested in vitro for its ability to specifically bind to an Ang-2 polypeptide or bind to and antagonize the Tie-2 receptor.

In yet another working example, candidate compounds may be screened for those that specifically modulate Ang-1 function. Preferred candidate compounds will increase (e.g., by at least 10%, 20%, 30%, 40%, 50%, 60%, 70%, 80%, 90%, or more) Ang-1 biological activity. Ang-1 biological activity can be readily assayed using any of the assays known in the art or described herein, including but not limited to assays for PI3K activity or p85 subunit phosphorylation, assays for Rac1 activation, assays for p190RhoGAP activation, and assays for RhoA inhibition.

In one particular working example, a candidate compound that binds to an Ang-2 polypeptide may be identified using a chromatography-based technique. For example, a recombinant Ang-2 may be purified by standard techniques from cells engineered to express Ang-2 and may be immobilized on a column. A solution of candidate compounds is then passed through the column, and a compound specific for the Ang-2 polypeptide is identified on the basis of its ability to bind to the polypeptide and be immobilized on the column. To isolate the compound, the column is washed to remove non-specifically bound molecules, and the compound of interest is then released from the column and collected. Similar methods may be used to isolate a compound bound to a polypeptide microarray. Compounds isolated by this method (or any other appropriate method) may, if desired, be further purified (e.g., by high performance liquid chromatography). In addition, these candidate compounds may be tested for their ability to decrease the biological activity of an Ang-2 polypeptide. Compounds isolated by this approach may also be used, for example, as therapeutics to treat or prevent a vascular leak disorder in a human subject. Compounds that are identified as binding to Ang-2 or Tie-2 with an affinity constant less than or equal to 10 mM are considered particularly useful in the invention. Alternatively, any in vivo protein interaction detection system, for example, any two-hybrid assay may be utilized to identify compounds or proteins that bind to a polypeptide of the invention.

### Identification of New Compounds or Extracts

In general, compounds capable of decreasing the activity of Ang-2 are identified from large libraries of both natural product or synthetic (or semi-synthetic) extracts or chemical libraries or from polypeptide or nucleic acid libraries, according to methods known in the art. Those skilled in the field of drug discovery and development will understand that the precise source of test extracts or compounds is not critical to the screening procedure(s) of the invention. Compounds used in screens may include known compounds (for example, known therapeutics used for other diseases or disorders). Alternatively, virtually any number of unknown chemical extracts or compounds can be screened using the methods described herein. Examples of such extracts or compounds include, but are not limited to, plant-, fungal-, prokaryotic- or animal-based extracts, fermentation broths, and synthetic compounds, as well as modification of existing compounds. Numerous methods are also available for generating random or directed synthesis (e.g., semi-synthesis or total synthesis) of any number of chemical compounds, including, but not limited to, saccharide-, lipid-, peptide-, and nucleic acid-based compounds. Synthetic compound libraries are commercially available from Brandon Associates (Merrimack, N.H.) and Aldrich Chemical (Milwaukee, Wis.). Alternatively, libraries of natural compounds in the form of bacterial, fungal, plant, and animal extracts are commercially available from a number of sources, including Biotics (Sussex, UK), Xenova (Slough, UK), Harbor Branch Oceangraphics Institute (Ft. Pierce, Fla.), and PharmaMar, U.S.A. (Cambridge, Mass.). In addition, natural and synthetically produced libraries are produced, if desired, according to methods known in the art, e.g., by standard extraction and fractionation methods. Furthermore, if desired, any library or compound is readily modified using standard chemical, physical, or biochemical methods.

In addition, those skilled in the art of drug discovery and development readily understand that methods for dereplication (e.g., taxonomic dereplication, biological dereplication, and chemical dereplication, or any combination thereof) or the elimination of replicates or repeats of materials already known for their molt-disrupting activity should be employed whenever possible.

When a crude extract is found to increase the biological activity of an Ang-2 polypeptide, or to bind to an Ang-2 polypeptide, further fractionation of the positive lead extract is necessary to isolate chemical constituents responsible for the observed effect. Thus, the goal of the extraction, fractionation, and purification process is the careful characterization and identification of a chemical entity within the crude extract that decreases the biological activity of an Ang-2 polypeptide. Methods of fractionation and purification of such heterogeneous extracts are known in the art. If desired, compounds shown to be useful as therapeutics for the treatment or prevention of a vascular leak disorder or hypotensive disorder are chemically modified according to methods known in the art.

### EXAMPLES

The examples below describe complementary human, murine, and in vitro experiments that demonstrate that Ang-2 is a mediator of vascular leak. Specifically, the experiments show that Ang-2 is significantly elevated in humans with sepsis who have impaired oxygenation and that serum from these patients disrupts endothelial architecture. This effect correlates with the measured increases in Ang-2 levels and abates with clinical improvement. The experiments also show that Ang-2 alone can provoke endothelial cell barrier disruption and also pulmonary leak and congestion in otherwise healthy adult mice. The experiments also show that the effects of Ang-2 on ECs are mediated by Rho kinase activation and myosin light chain phosphorylation. Taken together, the results identify Ang-2 as a both a biomarker of and a mediator of vascular leak syndromes.

### Example 1

### Circulating Ang-2 is Significantly Elevated Among Patients with Severe Sepsis

The baseline characteristics of a cohort of 22 patients identified prospectively by applying the standard definition of sepsis (see Methods) to screen weekday admissions to the ICU at Beth Israel Deaconess Medical Center over a two-month period in 2004 are shown in Table 1, below.

TABLE 1
Baseline characteristics of sepsis cohort.

| CHARACTERISTIC | VALUE |
|---|---|
| Number of patients | 22 |
| Age (yrs) | 69.1 ± 15.3 |
| Female sex | 11 (50%) |
| Survival to Discharge | 16 (73%) |
| APACHE II score | 21.2 ± 5.3 |
| Portal of Entry | |
| Lung | 6 (27%) |
| GU | 5 (23%) |
| Catheter | 3 (14%) |
| Abdominal | 4 (18%) |
| Other | 2 (9%) foot; 1(5%) endocarditis; 2 |
| | (9%) unknown |
| Maximum number of vasoactive | |
| agents used at any one time | |
| during hospitalization | |
| 0 | 6 (27%) |
| 1 | 7 (32%) |
| 2 | 6 (27%) |
| 3 | 3 (14%) |
| Prior/co-morbid conditions | |
| Coronary artery disease | 8 (36%) |
| Diabetes mellitus | 9 (41%) |
| Liver disease | 2 (9%) |
| COPD/asthma | 6 (27%) |
| Cancer | 7 (32%) |
| ESRD | 6 (27%) |

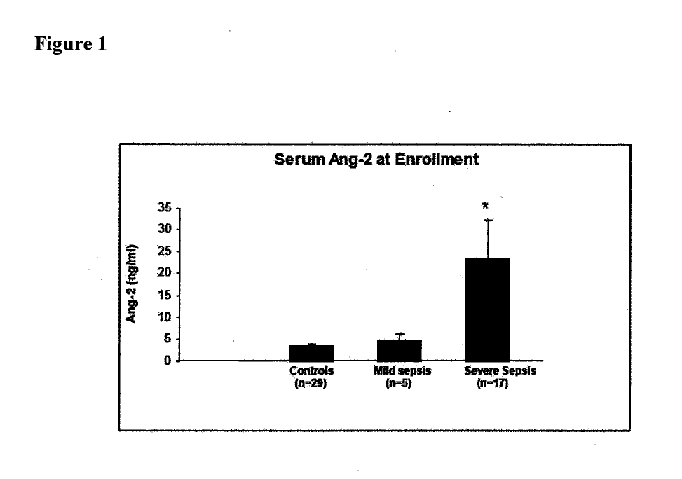

Control patients were selected from control hospitalized patients (n=29), with a variety of illnesses ranging from infectious (e.g., pyelonephritis, aseptic meningitis, pneumonia) to cardiovascular (e.g., angina, syncope) and neurologic (e.g., stroke) diseases. These patients were equally divided among male and female, were older (mean age 69.1±15.3 yrs) and had a high occurrence of co-morbid medical conditions. Portals of entry were varied as was the requirement for vasopressors. Discarded blood specimens were collected from the clinical laboratory in accordance with an IRB-approved protocol, and serum Ang-2 was measured with a commercially available ELISA. Serum and clinical data were also collected on a random selection of patients admitted to the general medical service without evidence of sepsis to serve as controls (FIG. 1, Controls, n=29). Ang-2 was significantly elevated at the time of study enrollment among those individuals with severe sepsis defined by the presence of shock or multi-organ dysfunction (23.2±9.1 ng/ml, n=17, p=0.0071) whereas those with mild sepsis (4.78±1.45 ng/ml, n=5) and control patients without sepsis (3.5±0.55 ng/ml, n=29) had statistically indistinguishable enrollment serum Ang-2 values (FIG. 1). These two less sick groups maintained stable serum Ang-2 values<10 ng/ml, whereas the severe sepsis group had a trend toward an even higher peak Ang-2 of 32.4±8.7 ng/ml during the course of their admissions. This strong correlation of Ang-2 elevation with severe sepsis was observed despite the small size of the sepsis cohort and its relative wellness—as reflected in the 73% survival rate to discharge and the mean entrance APACHE II score<22. Of note, serum Ang-1 was ˜1 ng/ml without significant differences between groups, and taking an Ang-2/Ang-1 ratio did not improve sensitivity.

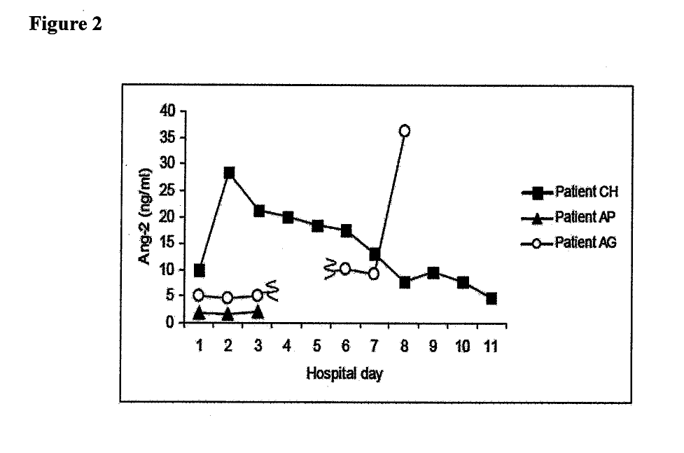

FIG. 2 shows the temporal trends of circulating Ang-2 in three illustrative hospitalized patients. Patient CH (FIG. 2, -▪-, severe sepsis and recovers), a 74 year-old woman, was admitted to the medical intensive care unit with severe enterococcal urosepsis, was treated with broad-spectrum antibiotics, initially required 3 vasoactive agents to manage shock, and was mechanically ventilated. Her nadir PaO₂/FiO₂=240 occurred on hospital day 2, correlating with peak circulating Ang-2. She progressively convalesced and was extubated prior to discharge. Patient AP (FIG. 2, -▴-, infection, no SIRS), a 92 year-old woman, was admitted to the general medicine service from a nursing home for increased confusion over her baseline dementia. She had no evidence of sepsis, shock, or respiratory compromise and PaO₂/FiO₂>300. She was treated for a foot wound infection with two antibiotics and was discharged in stable condition back to the nursing home. Patient AG (FIG. 2, -∘-, hypotension, no sepsis), a 77 year-old man, was first admitted to the general medicine service with hypotension following excessive fluid removal at hemodialysis—there was no evidence of infection, systemic inflammatory response or respiratory compromise with PaO₂/FiO₂>300 (hospital days 1-3). However, three months later (graphed as hospital days 6-8 for purposes of illustration), the same patient (FIG. 2, -∘-, severe sepsis) was re-admitted to the intensive care unit following emergent right leg amputation for gangrene complicated by shock and inability to extubate. Nadir PaO₂/FiO₂=144 occurred on the same day as peak Ang-2 (depicted as hospital day 8 for purposes of illustration), when he died despite full care.

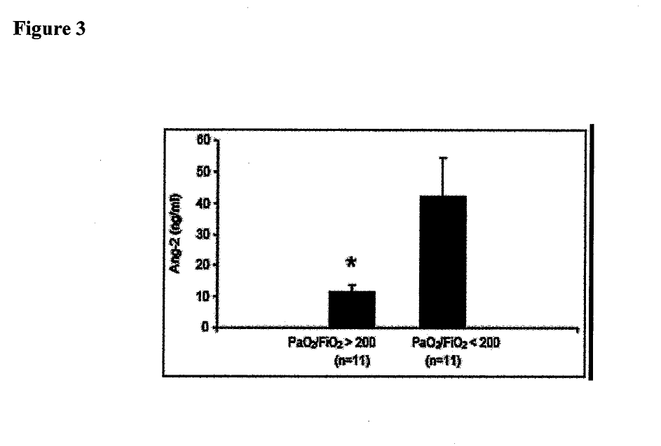

Our hypothesis was that an Ang-2 imbalance could occur in sepsis and that, should it occur, the lung, where Tie-2 expression is highest, would be preferentially affected. We compared peak circulating Ang-2 value during hospitalization between individuals with very poor oxygenation versus those with less impaired oxygenation. PaO₂/FiO₂, the ratio of arterial blood's oxygen partial pressure PaO₂, to the fraction of inspired air consisting of oxygen (FiO₂) was used as a metric to assess the defect in oxygen absorption from the lung into the bloodstream. After determining that the first part of our hypothesis (angiopoietin imbalance) was in agreement with human sepsis data, we next sought to assess whether the second part of our hypothesis (preferential effect on lung) would also be supported by human data. Therefore, we employed PaO₂/FiO₂ ratio to segregate sepsis subjects into more severe and less severe degrees of lung injury; a cutoff value of 200 was based on the consensus definition of ARDS (N. Engl. J. Med. 342: 1301-1308 (2000)). Again, despite the small sepsis cohort and its overall wellness, serum Ang-2 did correlate strongly with nadir impairment in gas exchange—41.95±12.44 ng/ml among those with PaO₂/FiO₂<200 versus 11.22±2.44 ng/ml in the better-oxygenated group (PaO₂/FiO₂>200); p=0.02 (FIG. 3). Among the 11 patients with PaO₂/FiO₂<200, only one met all criteria for ARDS; incidentally, he had the highest measured Ang-2 in our cohort at 139 ng/ml.

Ang-2 values were higher among those with APACHE II score≧25 (41.6±24.7 ng/ml, n=5) than those with APACHE II<25 (22.2±5.8 ng/ml, n=17), but this did not meet statistical significance. Ang-2 values did not differentiate survivors from non-survivors, and also did not correlate with liver dysfunction, a history of heart failure, or a history of renal insufficiency, at least in this small cohort.

### Example 2

### Serum from Human Subjects with Sepsis Disrupts Endothelial Architecture, an Effect that Abates with Clinical Convalescence and is Reversed by Ang-1

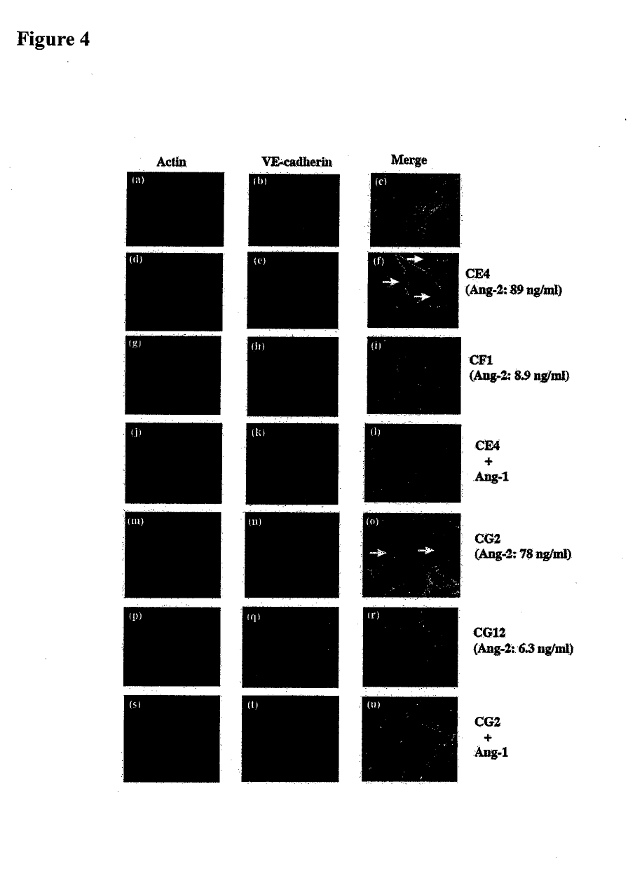

Separation of adjacent endothelial cells from one another leads to paracellular gap formation—a process driven by actin-myosin-based cell contraction (McDonald et al., Am J. Physiol. 266: L61-83 (1994), van Hinsbergh et al., J. Anat. 200: 549-560 (2002)). Such gaps permit para-endothelial movement of macromolecules and, thus, represent a structural change that correlates with hyperpermeability. To test what effect human serum had on cultured endothelial cells, we added serum from two patients, CE4 (high Ang-2) and CF1 (low Ang-2) to HMVECs and stained for F-actin and VE-cadherin, a structural protein that helps maintain intercellular junctions. Incubation of HMVECs with control medium (FBS/culture medium) resulted in a compact, confluent cell layer with thin actin filaments and localization of VE-cadherin to cell-cell junctions (FIGS. 4A-4C). However, addition of high Ang-2 serum (CE4, Ang-2=89 ng/ml) induced thick actin stress fibers and intercellular gap formation (arrows, FIGS. 4D-4F) whereas low Ang-2 serum (CF1, Ang-2=8.9 ng/ml) did not (FIGS. 4G-4I). The gap formation provoked by CE4's serum was reversed with addition of recombinant human Ang-1 (FIGS. 4J-4L).

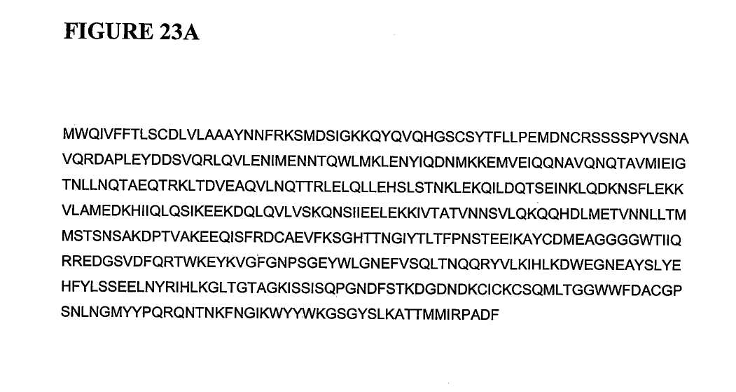

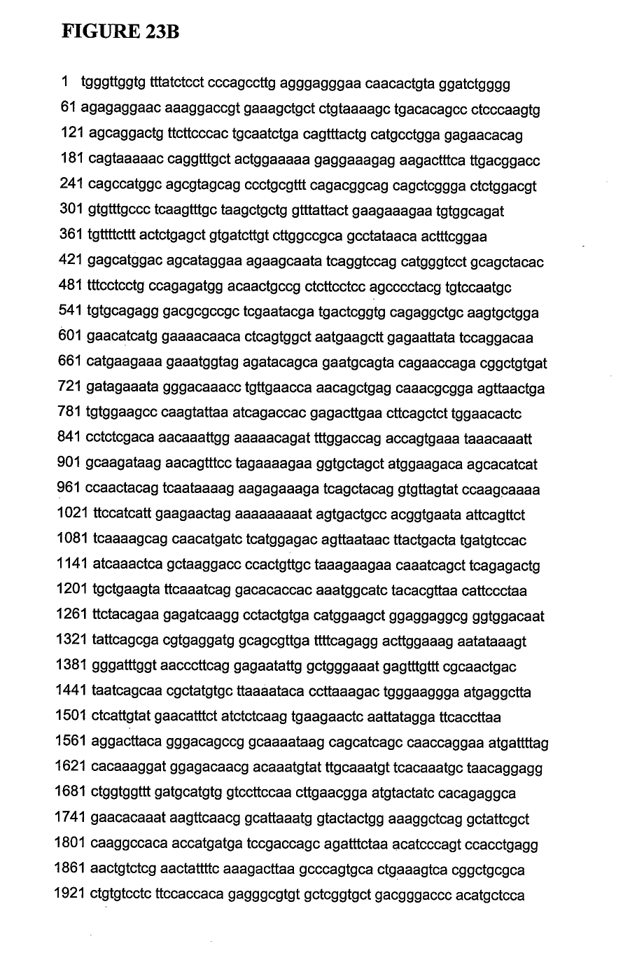

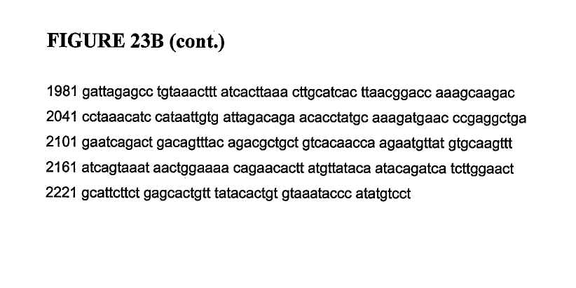

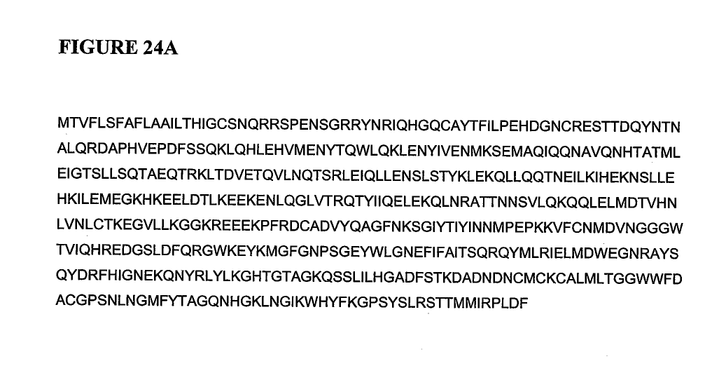

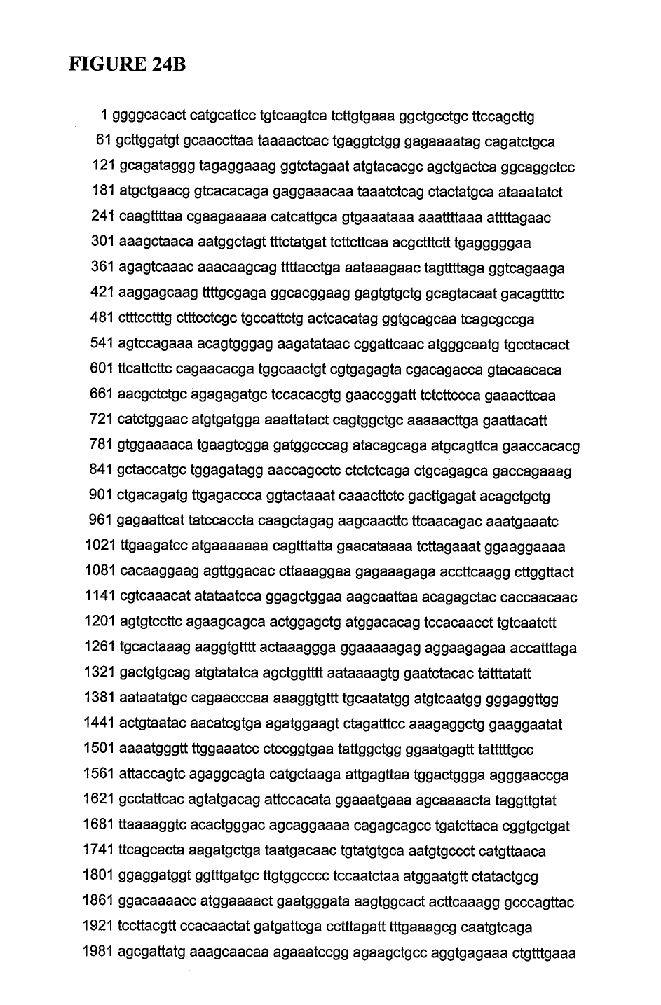

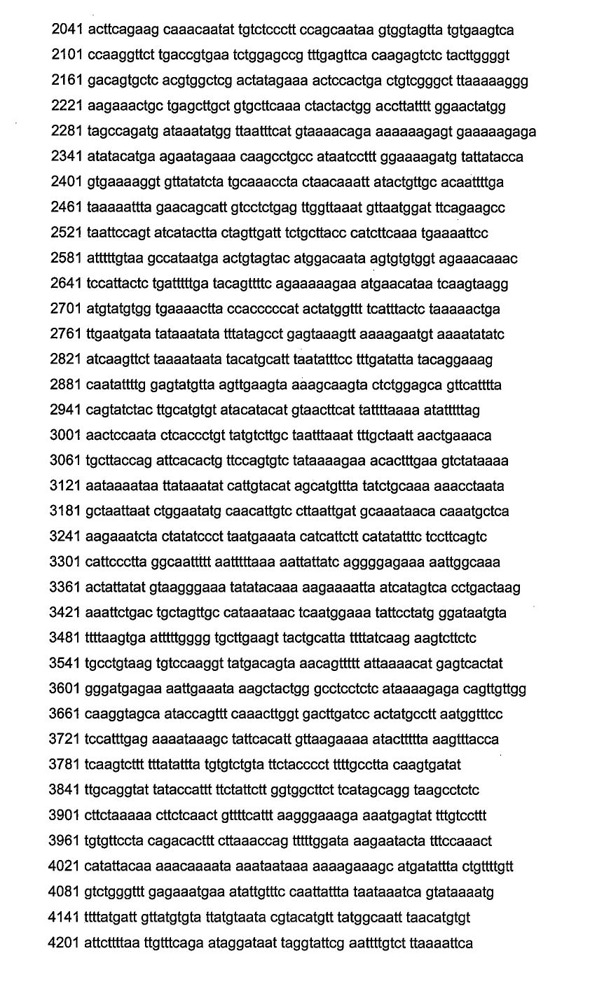

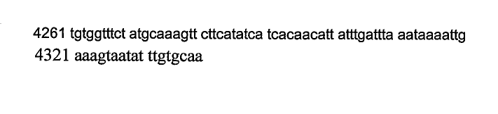

To address the potential biasing effect of unmeasured confounders between sera from two different patients, we repeated this experiment with serum from one patient taken at two time points during his hospitalization. CG2 was collected on hospital day 2 (Ang-2=78 ng/ml) and CG12 was collected on hospital day 16 (Ang-2=6.3 ng/ml). On hospital day 2, patient CG had PaO₂/FiO₂=56, was in septic shock, and had ARDS; by hospital day 16, patient CG was extubated, convalescing uneventfully and preparing for discharge. Serum from CG's 2^{nd} hospital day (CG2) induced gap formation and thick actin stress fibers (FIGS. 4M-40), effects not seen with his serum at discharge (CG12, FIGS. 4P-4R); moreover, effects of high Ang-2 serum from hospital day 2 (CG2) were reversed with addition of Ang-1 (FIGS. 4S-4U).

These results illustrate (a) the presence of a serum activity during severe sepsis that induces endothelial barrier disruption; (b) that clinical resolution correlates with decreased barrier-disrupting activity; and (c) that this activity can be reversed with Ang-1, suggesting that Ang-2 in human serum is at least partially responsible for altering endothelial architecture in sepsis.

### Example 3

### Ang-2 Alone Recapitulates the Effects of Sepsis Serum on Endothelial Architecture and Promotes Hyperpermeability

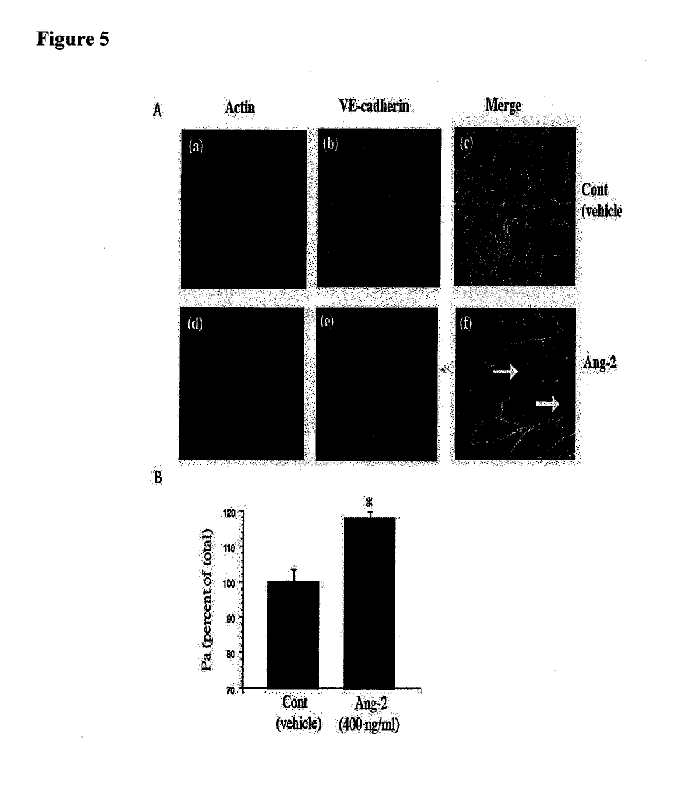

Having observed the effect of human serum on cultured endothelial cells described in Example 2, we next tested whether Ang-2 alone could reproduce disruption of endothelial architecture. Recombinant human Ang-2 (100 ng/ml) was added to HMVECs that were subsequently stained for F-actin and VE-cadherin. As suspected, Ang-2 induced the formation of thick actin stress fibers and intercellular gaps (FIG. 5A, panels d-f, arrows), effects not seen with vehicle incubation (FIG. 5, panels A-C). This experiment confirmed the hypothesis raised by the results in FIGS. 4A-4U—namely that Ang-2 alone could provoke potentially pathologic structural changes in endothelium.

We next confirmed that Ang-2 could modulate barrier function by measuring the clearance of FITC-labeled-albumin across a HMVEC monolayer with and without Ang-2 stimulation. FIG. 5G shows that Ang-2 stimulation for 8 hours increased permeability by approximately 20% compared with control (p<0.01), values comparable to the effect of endotoxin alone.

### Example 4

### The Gap-Formation Effect of Ang-2 on ECs is Mediated by Activation of Rho Kinase Leading to Myosin Light Chain Phosphorylation

Since Ang-2 appeared to be a likely mediator of endothelial barrier disruption in human sepsis serum, we next pursued the intracellular mechanism through which Ang-2 could distort endothelial shape and cell-cell contacts. Endothelial barrier function is known to be tightly regulated by myosin-driven cellular contraction (Wainwright et al., Proc. Natl. Acad. Sci. 100: 6233-6238 (2003), Garcia et al., J. Cell Physiol. 163: 510-522 (1995), Wysolmerski et al., Proc. Natl. Acad. Sci. 87:16-20 (1990), Sheldon et al., Am. J. Physiol. 265: L606-612 (1993)). For contraction to occur, myosin light chain (MLC) must be phosphorylated at Ser-19 by endothelial cell MLC kinase (EC MLCK), and phosphorylation of Ser-19 by EC MLCK is needed to activate actomyosin ATPase function (Ikebe et al., J. Biol. Chem. 260:10027-10031 (1985)), (Kamisoyama et al., Biochemistry 33:840-847 (1994)). We therefore hypothesized that Ang-2 upregulated MLC phosphorylation. Serum was taken from the same patients used for immunohistochemistry in FIGS. 4D-4I—CE2 (Ang-2=77 ng/ml) and CF5 (Ang-2=7.9 ng/ml)—and added to 24 hour serum-starved HMVECs.

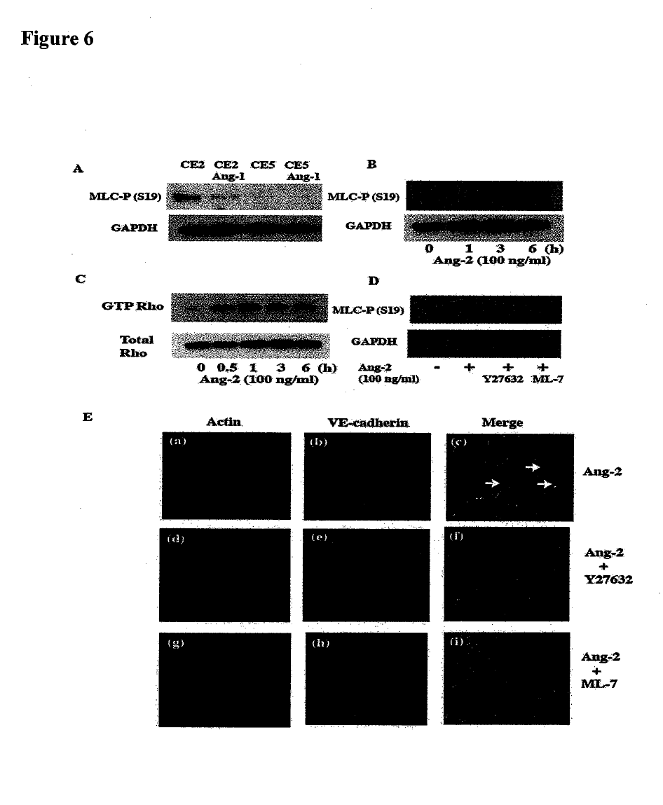

The high Ang-2 serum (CE2) caused MLC phosphorylation that was inhibited by addition of Ang-1 whereas the low Ang-2 serum led to markedly less MLC phosphorylation (FIG. 6A). MLC phosphorylation was elevated at 3 hours and 6 hours of stimulation with Ang-2 alone (FIG. 6B) and persisted for 24 hours.

Rho-GTPases play a pivotal role in the control of cellular actin rearrangement and cell shape (Hall et al., Science 279: 509-514 (1998)). Rho-kinase, a downstream target of RhoA, stimulates stress fiber formation by upregulating myosin light chain (MLC) phosphorylation through two mechanisms: (1) activation of EC MLCK and inhibition of myosin phosphatase activity (Amano et al., Science 271: 648-650 (1996), Kimura et al., Science 273: 245-248 (1996)).

Given this two-fold effect of RhoA on MLC, one would predict that a RhoA inhibitor would be even more potent than an MLCK inhibitor at blocking MLC phosphorylation. The following experiments addressed the question of whether Ang-2-induced MLC phosphorylation required activated RhoA.

Ang-2 (100 ng/ml) increased the active form of RhoA (Rho-GTP), peaking between 30 minutes and 1 hour (FIG. 6C). Pre-treatment of Ang-2-stimulated HMVECs with a specific inhibitor of Rho-kinase (Y27632, 10 μM) completely abolished Ang-2-induced phosphorylation of MLC (FIG. 6D, third lane), while an EC MLCK inhibitor (ML-7, 10 μM) partially inhibited MLC phosphorylation (FIG. 6D fourth lane). These results demonstrate that Ang-2 mediates MLC-phosphorylation in a RhoA-dependent fashion in human microvascular endothelial cells.

Y27632 (10 μM) completely reversed the formation of thick actin stress fibers and paracellular gaps induced by Ang-2 (FIG. 6E, panels a-f). ML-7 (10 μM) partially reversed the Ang-2 induced structural changes on actin and adherens junctions (FIG. 6E, panels g-i), consistent with the less potent effect of ML-7 versus Y27632 on MLC phosphorylation (FIG. 6D). These results demonstrate that the deleterious structural effects of Ang-2 on endothelial cells are mediated through Rho-kinase and MLCK. The effect of Ang-1, an Ang-2 antagonist, on inhibition of RhoA activity and the mechanism for this inhibitory effect is described in detail in Example 9.

### Example 5

### Ang-2 Reverses Tie-2 Activation and a Different Method of Blocking Tie-2 Action has the Same Effect on the Contractile State of ECs

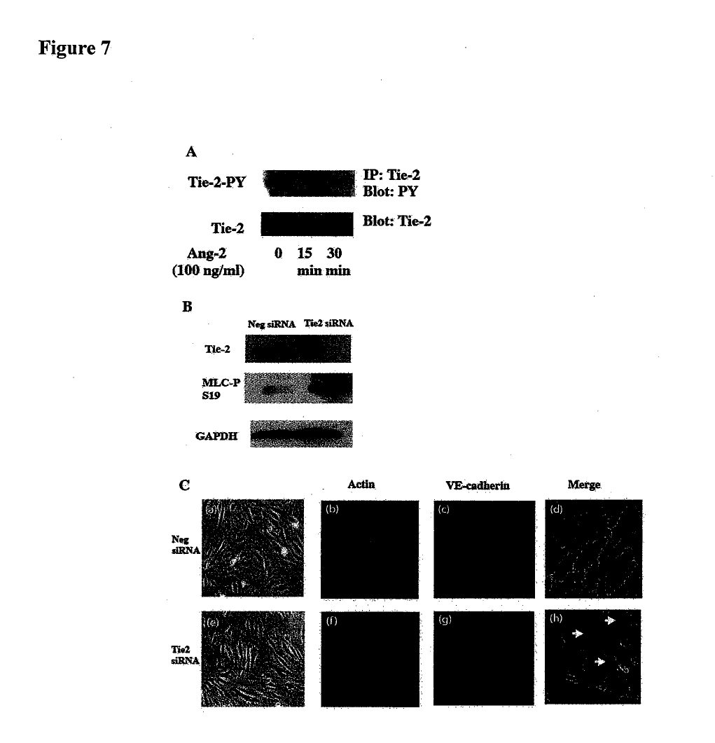

Multiple lines of evidence suggest that Ang-1 and Ang-2 are an agonist/antagonist pair at the Tie-2 receptor (Hanahan et al., Science 277: 48-50 (1997)). Ang-1 activates Tie-2, leading to receptor phosphorylation and subsequent signal transduction that promotes endothelial-cell survival and vessel assembly. Ang-2, on the other hand, is believed to act as a Tie-2 ligand that competitively binds the receptor and interferes with agonistic Ang-1/Tie-2 functions—i.e., Ang-2 binding of Tie-2 blocks its phosphorylation. Since this action of Ang-2 may be context, dose-, and duration-specific (Maisonpierre et al., Science 277: 55-60 (1997), Teichert-Kuliszewska et al., Cardiovasc. Res. 49: 659-670 (2001), Saharinen et al., J. Cell Biol. 169: 239-243 (2005)), we confirmed an inhibitory effect in HMVECs stimulated with Ang-2 (100 ng/ml) (FIG. 7A).

We then assessed the effect of Tie-2 signaling on MLC phosphorylation by using siRNA against Tie-2 receptor (Tie-2-siRNA). Tie-2-siRNA induced robust MLC phosphorylation (FIG. 7B middle), recalling the effect seen with Ang-2 treatment (FIG. 6B). Tie-2-siRNA caused a spindle phenotype (FIG. 7C, panel e), thick actin stress fibers and paracellular gap formation (FIG. 7C, panels f-h, arrows), effects not observed with negative control siRNA transfection (FIG. 7C, panels a-d). These morphologic changes are on the same spectrum, but even more severe, compared to those seen with addition of Ang-2 (FIGS. 5 D-F). These results suggest that Tie-2 signaling is constitutively active in this system. Addition of Ang-2 blocks Tie-2 signaling, leading, in turn, to Rho-kinase activation and MLC-phosphorylation with the end-result being endothelial cell contraction, gap formation and disruption of barrier integrity.

### Example 6

### Ang-2 Administration to Healthy Adult Mice Promotes Vascular Leak and Pulmonary Injury

We hypothesized that systemic administration of Ang-2 would provoke pulmonary vascular hyperpermeability and congestion. Evans blue avidly binds to serum albumin and can therefore be used as a tracer for trans-capillary flux of macromolecules. The extravasation of Evans blue has frequently been employed to quantify in vivo vascular permeability (Rinkema et al., J. Pharmacol. Exp. Ther. 230: 550-557 (1984), Green et al., J. Lab. Clin. Med. 111: 173-183 (1988)). Given the several limitations of in vitro permeability assays—e.g., lack of flow and variable hydrostatic pressure, used of cultured cells, absence of microenvironment, and absence of interacting cell types such PMNs (Weis et al., Nature 437: 497-504 (2005))—we felt that it was important to confirm leak across intact blood vessels in an in vivo setting. Adult mice were pre-treated (16 hour prior to Evans blue administration) with either vehicle or Ang-2 (10 μg) injected intraperitoneally prior to Evans blue dye injection and sacrifice.

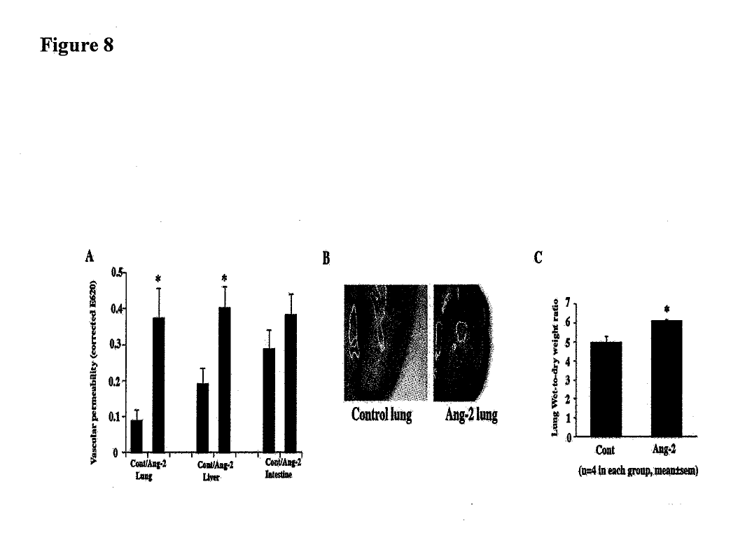

Spectrophotometric quantification of extravasated dye showed enhanced leakage, with a threefold increase in lungs and a twofold increase in livers of Ang-2-treated mice compared to vehicle-treated mice (FIG. 8A) (p<0.01). Intestines showed a trend toward increased permeability (FIG. 8A).

After washout of intravascular Evans blue by perfusing PBS through the right ventricle and venting from the vena cava, lungs of vehicle-treated mice were blanched-appearing (FIG. 8B, left); however, lungs of Ang-2-treated mice appeared more congested and purple-tinted (FIG. 8B, right), suggesting retention of dye in the extravascular space. In addition, the lung wet/dry weight (W/D) ratio increased from 5.01±0.26 to 6.13±0.03 with Ang-2 treatment (FIG. 8C) (P<0.01), suggesting increased lung water accumulation following Ang-2 administration.

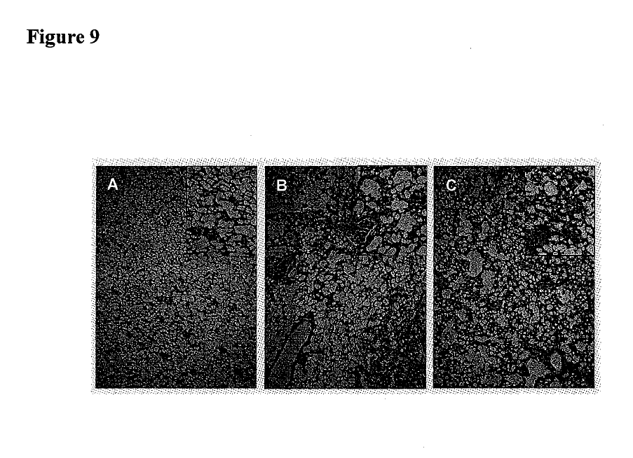

FIG. 9A shows lung from a control mouse injected with vehicle—alveolar septa form a fine, thin network (inset). 3 hour after systemic Ang-2 administration (10 μm), there is an increase in congestion and early extravasation into the interstitium (FIG. 9B). These changes are even more pronounced at 48 hours (FIG. 9C) following a total Ang-2 dose of 20 μg. These results show that excess Ang-2 is sufficient to promote pulmonary vascular leak and further substantiates the in vitro permeability effects observed earlier with Ang-2 stimulation (FIG. 5A, panels a-f). Moreover, the in vivo experiments suggest rapid and progressive lung injury with increasing amount and duration of systemic Ang-2 exposure.

### Example 7

### Ang-2 Mediated Contraction May Require Active NF-κB

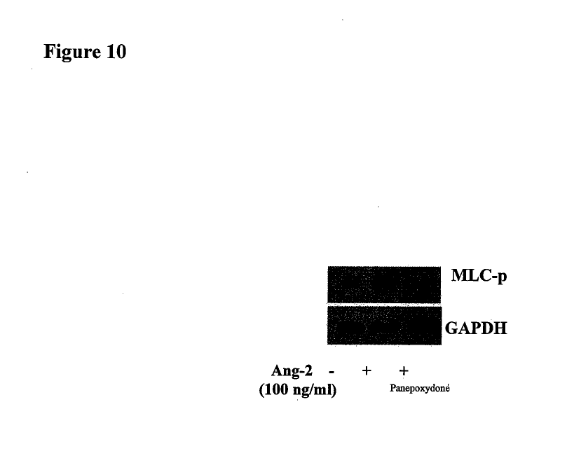

Acute phase reactants (e.g., TNFα, IL-1, and IL-6) and bacterial products, including DNA and cell wall components, are known to bind cell-surface receptors that trigger NF-κB activation. NF-κB transcriptional targets include an array of proteins involved in cell proliferation, adhesion, and immune activation. This pathway is widely accepted as central in the host response to infection (LiQ et al., Nat. Rev. Immunol. 2: 725-734 (2002)), particularly within phagocytes, but also in ECs (Pober et al., Ciba Found Symp. 131: 170-184 (1987), Iademarco et al., J. Biol. Chem. 267: 16323-16329 (1992)). We therefore hypothesized that NF-κB activation may be necessary for the permeability response of ECs to Ang-2. Panepoxydone is a fungally derived compound that prevents nuclear translocation of NF-κB, thereby blocking its function. When added to EC monolayers, we observed marked inhibition of Ang-2 mediated MLC phosphorylation (FIG. 10, lane 3) despite overloading of that lane. This result suggests a novel intersection linking Tie-2 signaling, NF-κB activation, and MLC phosphorylation.

### Example 8

### IL-2 Mediated Vascular Leak Correlates with Circulating Ang-2

High-dose interleukin-2 (HD IL-2) is the only FDA-approved therapy for metastatic renal cell cancer and is also used as salvage therapy in patients with metastatic melanoma. HD IL-2 is believed to activate a patient's own T lymphocytes and NK cells to attack existing tumor. Though the response rate is ˜10%, those who do improve have durable response measurable in years.

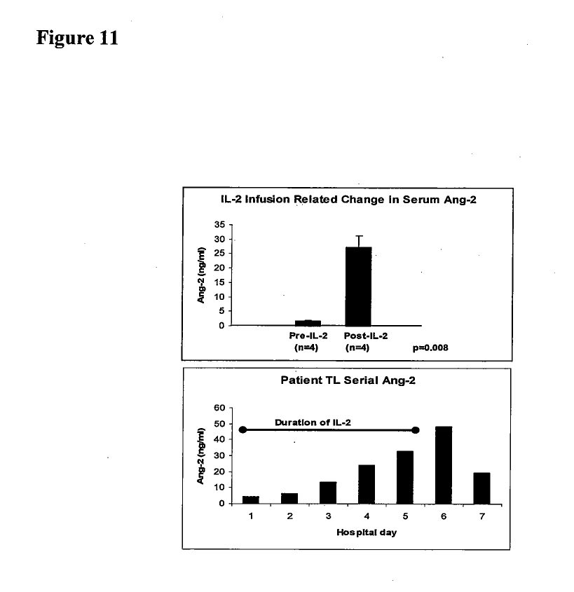

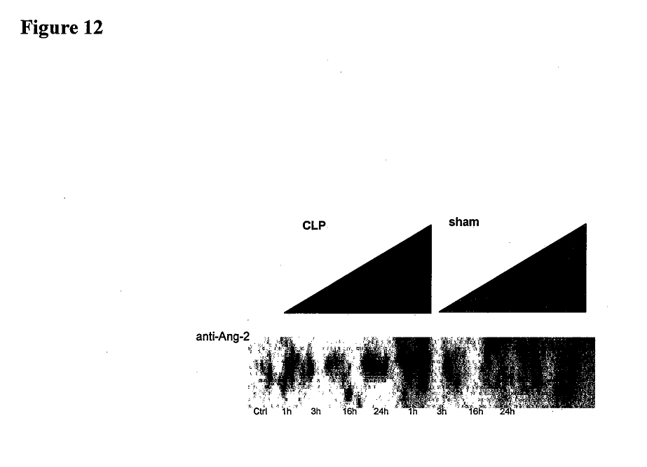

Up to 65% of subjects receiving HD IL-2 develop a dose-limiting vascular leak syndrome characterized by marked hyperpermeability leading to diffuse extravasation of fluid, particularly in the lung, where it can provoke respiratory distress (Lee et al., J. Clin. Oncol. 7: 7-20 (1989)). We hypothesized that serum Ang-2 would be elevated following HD IL-2, and indeed, we saw marked elevation following therapy (FIG. 11, top, 1.72±0.22 ng/ml vs. 27.14±4.06 ng/ml, n=4, p=0.008). Similarly, serial Ang-2 was measured in one of these patients during and after a 5-day infusion of IL-2. Each day of infusion was associated with progressively rising serum Ang-2, followed by rapid decline over 24 hours from a peak of 48.59 ng/ml on day 6 to 19.21 ng/ml by day 7 (FIG. 11, bottom). The role of Ang-2 in vascular leak disorders associated with HD IL-2 therapy is further described in Example 14, below.

### Example 9

### Rodent Models of Sepsis

Cecal ligation and perforation (CLP) is a model of intraabdominal sepsis that faithfully mimics the temporal profile of cytokines and progressive physiologic changes seen in human sepsis; as such, despite the surgical nature of the insult and the inter-investigator variability in strain use and technique, it has become one preferred model for studying sepsis in vivo (Marshall et al., Shock 1: 1-6 (2005)). Using this CLP animal model, we found a time-dependent rise in circulating Ang-2 following CLP but none following sham operation. Similar results were obtained 24 hour after intraperitoneal injection of endotoxin, which is another useful animal model for studying sepsis in vivo. The CLP model in male C57b16 mice can be used as an animal model to study any of the Ang-2 antagonists described herein, for example to determine efficacy or therapeutic dosages of potential Ang-2 antagonist compounds.

### Materials and Methods

The following materials and methods were used in the experiments described above.

### Human Subjects:

For the pilot sepsis study, weekday admissions to the BIDMC intensive care unit were screened daily for a two-month period and enrolled if they met criteria for sepsis (SIRS+evidence of infection). SIRS was defined by the presence of 2 of the following 4: (1) temperature<36° C. or >38° C.; (2) heart rate>90 beats/minute; (3) respiratory rate>20 breaths/minute or PaCO₂<32 mmHg; (4) white blood cell count>12,000 cells/mm', <4000 cells/mm³, or >10% immature forms. Severe sepsis was defined by the presence of shock or multi-organ dysfunction. Serum was aliquoted in a sterile fashion into cryo-vial tubes and stored at −80° C. prior to ELISA. Stable Ang-2 signal was confirmed through several freeze-thaw cycles. All data were encrypted to protect patient privacy. For the pilot HD IL-2 study, patients receiving HD IL-2 provided informed consent permitting access to serum and clinical data which were processed as outlined above. Serum and clinical data were collected in accordance with BIDMC IRB approved protocols.

### ELISA:

Ang-2 was measured in patient serum by sandwich ELISA using the reagents and protocol supplied with the human Ang-2 ELISA kit (R&D systems, Minneapolis, Minn.).

### Chemicals:

Human recombinant angiopoietin-1 (Ang-1) and angiopoietin-2 (Ang-2) were purchased from R&D systems (Minneapolis, Minn.). The RhoA-associated protein kinase inhibitor Y-27632 and myosin light chain kinase (EC MLCK) inhibitor ML-7 were purchased from EMD Biosciences Inc. (San Diego, Calif.). Other reagents were obtained from Sigma (St Louis, Mo.).

### Cell Culture:

Human microvascular endothelial cells from human lung or neonatal dermis (HMVEC-p or HMVEC-d) (Cambrex Bio Science Walkersville, Inc., Walkersville, Md.) were cultured in EBM-2 (Cambrex Bio Science Walkersville, Inc.) supplemented with 5% fetal bovine serum (FBS) and growth factors according to the manufacturer's instructions. Serum starvation was performed by incubation in 0.25% FBS/EBM-2 for 24 hours.

### Animals:

Male C57b16 (Jackson Lab, Bar Harbor, Me.) weighing 18-25 g were used, animals were allowed to acclimate for one week prior to experiments, and all animal experiments have been approved by the BIDMC IACUC.

### In Vivo Sepsis Models:

(a) CLP was performed on male C57b16 mice in collaboration with Per Olof Hasselgren. Animals were anesthetized with Avertin (2,2,2-tribromoethanol). After midline laparotomy, the cecum was identified and ligated with 3.0 silk suture distal to the ileocecal valve to prevent intestinal obstruction. A 20 g needle was used to puncture the cecal tip once and 1 mm stool was expressed from the perforation. The bowels were then returned to the abdominal cavity, a two-layered closure was performed, and animals were injected with 200 μl saline for resuscitation. In other models, (b) endotoxin (Sigma, strain O111:B4) 10 mg/kg was injected intraperitoneally after sterile preparation of the abdominal wall.

### Evans Blue Permeability Assay:

Mice were injected with 10 μg of Ang-2 or vehicle intraperitoneal (i.p.) and, after 16 hours, were anesthetized with Avertin (2,2,2-tribromoethanol). 2% Evans blue (50 μl) was then injected into the retro-orbital sinus. (In preliminary experiments with control mice, n=6, we confirmed that the retro-orbital sinus provided a route of intravascular injection that allowed near-100% delivery of Evans blue in a reproducible fashion.) 10 minutes after Evans blue injection, mice were sacrificed and perfused with PBS for 10 minutes through a cannula placed in the right ventricle. Blood and PBS were vented through an incision in the vena cava. After 10 minutes of perfusing the right ventricle with PBS, the outflow from the vena cava was observed to be clear, confirming that blood (and intravascular Evans blue) had been flushed out of the circulation. Washout of intravascular contents was also confirmed histologically after 10 minutes PBS perfusion. After homogenization in 1.5 ml formamide, Evans blue was extracted by incubating the samples at 70° C. for 24 hours, and the concentration of Evans blue was estimated by dual-wavelength spectroscopy to correct for heme E_{620 nm} (corrected)=E_{620 nm}−(1.426*E_{740 nm}+0.030).

### Lung Wet-to-Dry Weight Ratio:

Lung wet weight (W) was determined immediately after removal of the right lung. Lung dry weight (D) was determined after the lung had been dried in an oven at 50° C. for 24 hours. The W/D ratio was calculated by dividing the wet weight by the dry weight.

### Histology:

Lungs were harvested, fixed in 10% formalin, embedded in paraffin, sectioned, and stained with hematoxylin and eosin.

### Western Blot Analysis:

Cells were washed with ice-cold PBS three times and lysed with ice-cold RIPA buffer (50 mM Tris-HCl pH7.4, 150 mM NaCl, 1% NP-40, 0.5% Sodium Deoxycholate, 0.1% SDS and 1 mM EDTA) supplemented with protease inhibitors (Roche Diagnostics, Indianapolis, Ind.) and 10 mM NaF. Protein concentrations were determined by Bradford protein assay with bovine serum albumin as a standard (Bio-Rad, Hercules, Calif.). Primary antibodies were obtained from these suppliers: anti-Ang-2 polyclonal antibody form Santa Cruz biotechnologies, anti-Tie2 antibody (clone Ab33) was from Upstate Cell Signaling Solutions (Lake Placid, N.Y.); anti-GAPDH monoclonal antibody was from Chemicon International (Temecula, Calif.).

### Immunoprecipitation:

200 μg of total protein were incubated with anti-Tie2 antibody for 3 hours, followed by incubation with protein A sepharose (Zymed, San Francisco, Calif.) for 2 hours at 4° C. After washing the beads, proteins were eluted by heating in SDS-sample buffer and detected by Western blot analysis with Anti-phospho-tyrosine (clone 4G10, Upstate Cell Signaling Solutions) as described before.

### MLC Phosphorylation Assay:

After signal starvation with 0.25% FBS EBM-2 for 24 h, cells were treated with 100 ng/ml Ang-2 or vehicle for 0, 1, 3, and 6 hours. Phosphorylated myosin light chain (MLC-p) (phospho-serine 19) and GAPDH were detected by Western blot analysis. For human subject serum effects on HMVECs, serum was diluted to 5% with EBM-2 and filtered with low protein binding PVDF membrane (0.22 μm, Millipore Corp, Bedford, Mass.). Anti-MLC-phospho-serine-19 Ab was obtained from Abcam Inc.

### Rho Activity Pull-Down Assay:

RhoA activity assay was performed and quantified using the RhoA activation assay kit according to the manufacturer's instruction (Cytoskeleton, Denver, Colo.). Lysates from control and Ang-2-treated cells containing equivalent protein concentrations were rotated for 60 minutes with 40 μl slurry of a GST-fusion protein composed of the Rho-binding domain of the specific RhoA effector rhotekin coupled to agarose beads. Beads were collected by centrifugation and washed three times with lysis buffer. Whole cell lysates from both control and Ang-2-treated cells were also run to determine baseline levels of total RhoA protein. Separated proteins were transferred to PVDF and immunoblotted with a monoclonal antibody to RhoA (Santa Cruz Biotechnology, Santa Cruz, Calif.).

### Immunofluorescence:

HMVEC were grown to confluence on glass coverslips coated with 1% gelatin. The cells were fixed for 10 minutes in 4% paraformaldehyde in PBS, incubated for 5 min in 0.5% Triton X-100 in PBS. After blocking, the monolayers were processed for staining with anti-VE-cadherin monoclonal antibody (BD Pharmingen, San Diego, Calif.) and Alexa Fluoro 488 goat anti-mouse IgG, rhodamine phalloidin (Molecular Probe, Eugene, Oreg.) for F-actin staining and TOPRO-3-iodine (Molecular Probe) for nuclear staining. Fluorescence images were obtained using a Bio Rad MRC confocal fluorescence microscope. For human subject serum experiments, serum was diluted to 10% with EBM-2 and filtered with low protein binding PVDF membrane (0.22 μm, Millipore Corp) prior to application on endothelial cell monolayers.

### FITC-Albumin Permeability Assay:

HMVEC monolayer permeability was determined with the use of FITC-labeled bovine serum albumin (Sigma) as described elsewhere (Tinsley et al., Am. J. Physiol Cell Physiol. 279: C1285-1289 (2000)). Coster Transwell membranes (Corning Inc. Corning, N.Y.) were coated with fibronectin and cells were grown until confluence. Vehicle or Ang-2 (400 ng/ml) with FITC-albumin (final 1 mg/ml) was added to the luminal chamber for 8 hours, and samples were taken from both the luminal and abluminal chamber for fluorometry analysis. The readings were converted with the use of a standard curve to albumin concentration. These concentrations were then used in the following equation to determine the permeability coefficient of albumin (Pa).

$${P}_{a} = \frac{[ A ]}{t} \times \frac{1}{A} \times \frac{V}{[ L ]} \text{?}$$

$$\text{?} \text{indicates text missing or illegible when filed}$$

where [A] is abluminal concentration; t is time in seconds; A is area of membrane in cm2; V is volume of abluminal chamber; and [L] is luminal concentration. siRNA transfection of endothelial cells: HMVECs were seeded on 10 cm dishes for Western blot (or on 1% gelatin-coated coverslips for immunohistochemistry experiments) 24 hour before experiments. Twenty μmol of validated, annealed small interfering RNA (siRNA) (Ambion, Inc. Austin Tex.) directed to human Tie-2 was transfected using silentFect Lipid reagent (Bio-Rad) according to manufacturer's instructions. Three days after transfection, cells were used for experiments. Down-regulation of Tie-2 receptor was verified by Western blotting with anti-Tie-2 polyclonal antibody (Upstate Cell Signaling Solutions).

### NF-κB Nuclear Translocation:

Ang-2 (100 ng/ml)-treated HMVEC with or without panepoxydone (5 mcg/ml) were scraped into ice-cold PBS and separated into nuclear and cytoplasmic fractions with a commercially available kit per the manufacturer's instructions (Active Motif, Carlsbad, Calif.) and NF-κB activity was measured by TransAM ELISA kit (Active Motif).

### Example 10

### Ang-1 Induces Rac1 Activation and RhoA Inhibition

The examples described above demonstrated that circulating levels of Ang-2 become elevated in human subjects with sepsis and that Ang-2 induces RhoA-mediated endothelial cytoskeletal changes that promote permeability. These experiments suggest that activation of Tie-2 by an Ang-2 antagonist, for example Ang-1, can protect against permeability by remodeling endothelial cytoskeletal forces and architecture.

Tie-2, when stimulated by Ang-1, is known to signal a pro-survival effect on endothelial cells through phosphoinositide 3 kinase (PI3K). PI3K generates phosphatidylinositol (3,4,5)-triphosphate (PtdIns (3,4,5) P3), which targets numerous effectors, including protein kinase B (Akt), phospholipases, and guanine-nucleotide exchange factors (GEFs) that activate Rho GTPases (Welch et al., FEBS Lett 546:93-97 (2003), Wymann et al., Curr. Opin. Cell Biol. 17:141-149 (2005)). Two members of the Rho family, RhoA and Rac1, have opposite effects on cells—the former induces actin stress fibers that increase centripetal tension and cause cell contraction whereas the latter is required to maintain adherens and tight junctions between cells (Burridge et al., Cell 116:167-179 (2004)). The role of p190RhoGAP is less well understood, but may be involved in restoration of endothelial barrier defense (Holinstat et al., J. Biol. Chem. 281:2296-2305 (2006)).

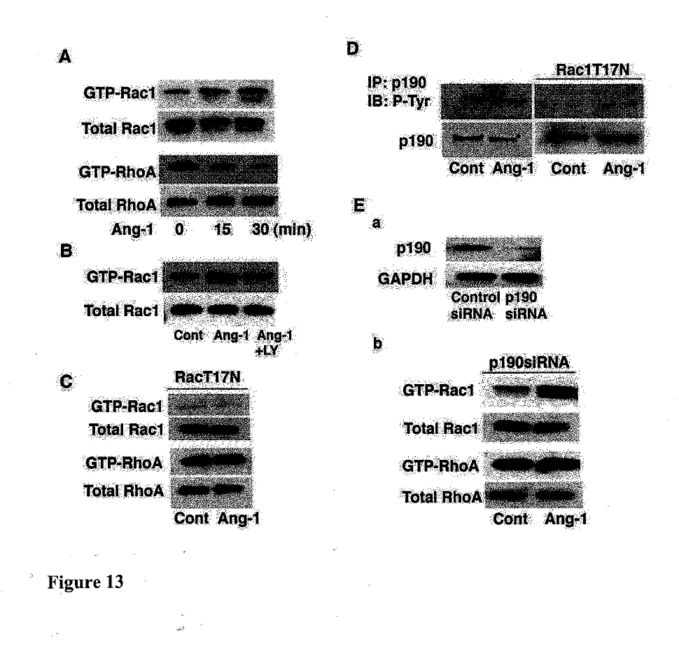

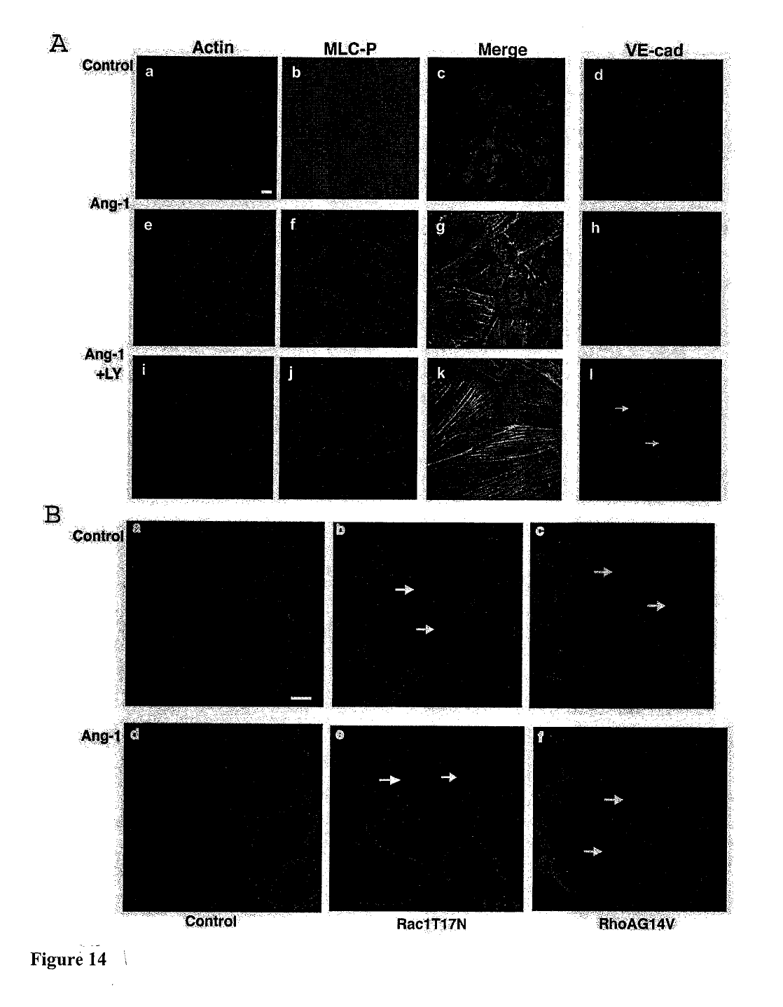

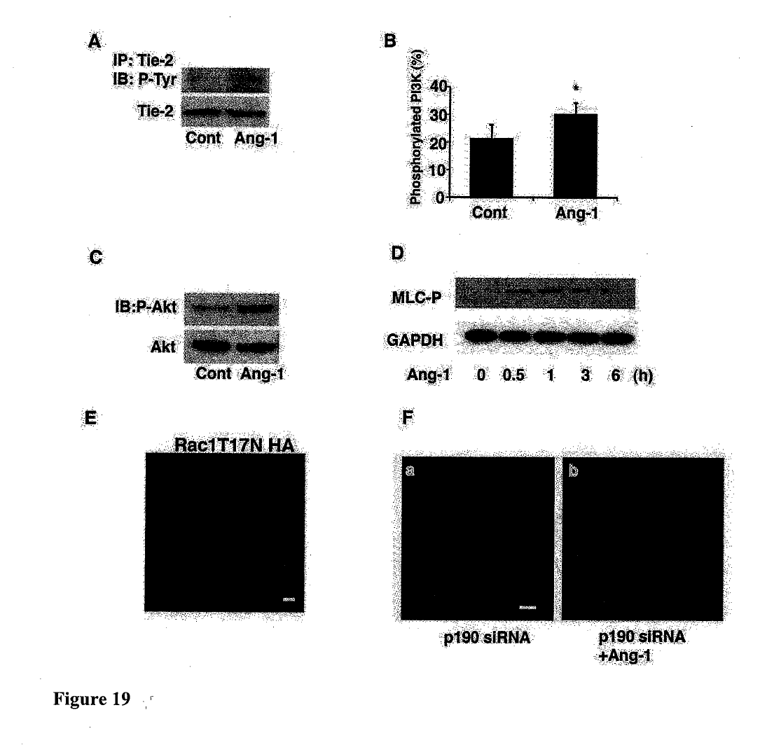

Rac and Rho, when transfected into human umbilical vein endothelial cells, can have antagonistic roles in regulating endothelial permeability responses to thrombin and histamine (Wojciak-Stothard et al., J. Cell Sci. 114:1343-1355 (2001)). Therefore, we examined whether Ang-1 affects the activities of endogenous Rac1 and RhoA in human lung microvascular ECs (HMVEC-L). Using a pull-down assay, we found that Rac1 activity was increased within 15 minutes following treatment with Ang-1 (FIG. 13A, upper panel) while RhoA activity was decreased 30 minutes after Ang-1 addition (FIG. 13A, lower panel). After establishing that Ang-1 activates Tie-2, PI3K, and Akt in HMVEC-L (FIGS. 19A-C), we found that the PI3K inhibitor LY294002 (10 μM) blocked Ang-1-induced activation of Rac1 (FIG. 13B), suggesting that PI3K is required for Ang-1 to positively regulate Rac1.

Since Rac1 is known to downregulate RhoA activity through p190RhoGAP in HeLa cells (Nimnual et al., Nat. Cell Biol. 5:236-241 (2003)), we next explored whether p190RhoGAP mediates similar cross-talk among these Rho family GTPases within ECs when they are stimulated by Ang-1. We found that Ang-1 could no longer suppress RhoA activity when HMVEC-L were transfected with a dominant negative form of Rac1 (Rac1T17N) using a lentiviral vector (FIG. 13C compared to FIG. 13A, lower panel). Transfection with Rac1T17N also diminished Ang-1 induced phosphorylation of p190RhoGAP (FIG. 13D).

To study the role of p190RhoGAP further, we used specific siRNA that blocked its expression by 90% (FIG. 13E, panel a). Suppression of p190RhoGAP did not affect Ang-1-induced Rac1 activation (FIG. 13E, upper panel b), but did abolish Ang-1-induced RhoA inhibition (FIG. 13E, lower panel b), analogous to the effect of Ang-1 on RhoA in the setting of dominant negative Rac1 (FIG. 13C). These results suggest that endogenous Rac1 and p190RhoGAP, activated by Ang-1, inhibit RhoA, and that p190RhoGAP acts downstream of activated Rac1.

### Example 11

### Preservation of Endothelial Junctions by Ang-1 Requires PI3K, Rac1, and p190RhoGAP

Rac1 and RhoA are critical regulators of actin polymerization and cytoskeletal tension. EC permeability can be promoted by centripetal force on the actin cytoskeleton and resisted by cell-cell adhesion mediated by VE-cadherin, a transmembrane protein that maintains endothelial adherens junctions (Dudek et al., J. Appl. Physiol. 91:1487-1500 (2001)). Phosphorylation of myosin light chain (MLC) increases actin-myosin cross-bridge formation and therefore plays a central role in regulation of endothelial permeability (Dudek et al., supra). Western analysis revealed that Ang-1 induced MLC phosphorylation (MLC-P), peaking at 0.5-1.0 h after stimulation (FIG. 19D). This correlated with an increase in cortical actin, peripheral MLC-P, and increased VE-cadherin staining at cell junctions compared to control cells when confluent HMVEC-L were analyzed by immunofluorescence microscopy (FIG. 14A, panels a-h). As expected, LY294002 reversed these effects of Ang-1 as evidenced by disruption of cortical actin, formation of thick central actin stress fiber bundles containing MLC-P, and development of intercellular gaps with attenuated VE-cadherin staining (FIG. 14A, panels i-l). These result show that inhibition of PI3K signaling in ECs can induce the structural changes associated with cellular contraction and loss of cell-cell adhesion.

Having observed that Ang-1 reciprocally regulates Rac1 and RhoA, we next studied the effect of Rac1T17N or a constitutively active RhoA (RhoAG14V) on endothelial architecture. Lentiviral delivery of Rac1T17N or RhoAG14V produced intercellular gaps (FIG. 14B, panels b-c) compared to control-virus-infected cells (FIG. 14B, panel a). In the presence of control virus, Ang-1 retained the ability to augment junctional VE-cadherin staining (FIG. 14B, panel d). However, this effect of Ang-1 was markedly diminished in Rac1T17N- and RhoAG14V-delivered cells, resulting in gap formation (FIG. 14B, panels e-f). These results show that Rac1 and RhoA have counteracting effects on endothelial cytoskeletal architecture and intercellular gap formation. Inhibition of endogenous Rac1 or activation of RhoA is sufficient to prevent Ang-1 mediated junctional fortification.

Since Ang-1 inhibits RhoA activity through Rac1-p190RhoGAP signaling (FIGS. 13C-E), we next tested the impact of p190RhoGAP on endothelial architecture. Its inhibition did not attenuate junctional VE-cadherin staining nor promote intercellular gap formation (FIG. 19F, panel a). Moreover, Ang-1 retained the ability to increase junctional VE-cadherin concentration (FIG. 19F, panel b). These results show that suppression of p190RhoGAP does not impede the major cytoskeletal action of Ang-1, potentially because Rac1 is upstream and thus has a p190RhoGAP-independent effect to strengthen cell junctions. These results also suggest that de-suppression of RhoA activity (by p190RhoGAP inhibition) is, alone, insufficient to disrupt junctional VE-cadherin or to induce gap formation.

### Example 12

### Ang-1 Requires Rac1 and p190RhoGAP to Block Endotoxin-Induced Structural Disruption

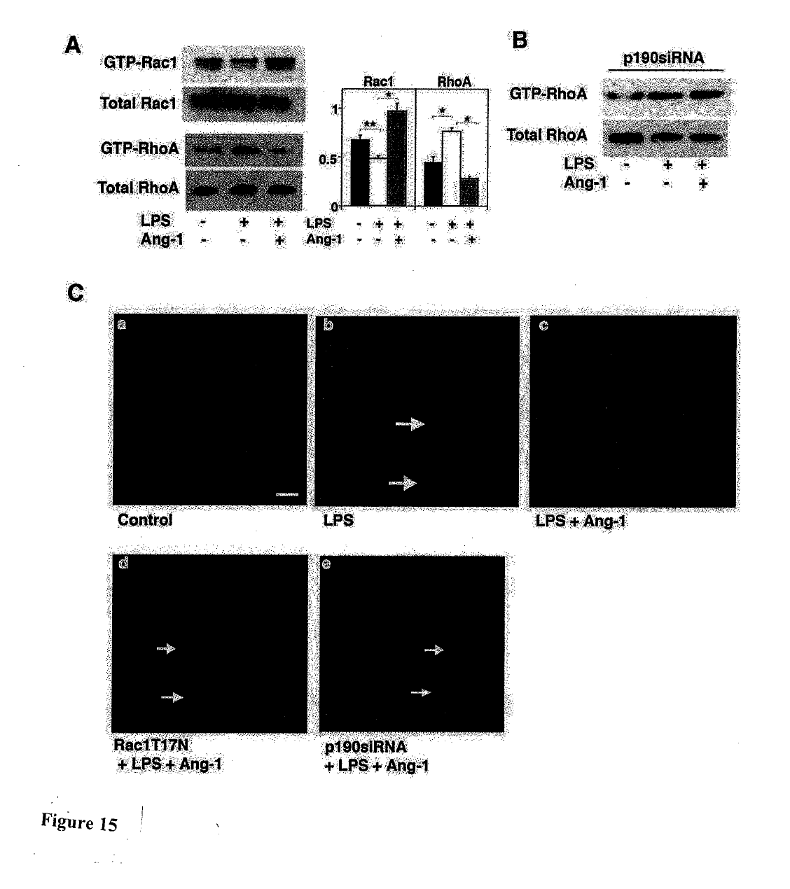

Endotoxin appears to induce disruption of the endothelial cytoskeleton and vascular permeability by activating RhoA (Essler et al., J. Immunol. 164:6543-6549 (2000), Thorlacius et al., J. Leukoc. Biol. 79:923-931 (2006)). We found that endotoxin treatment (100 ng/ml) of HMVEC-L mildly decreased Rac1 activity and strongly induced RhoA activity. Both of these effects were reversed by co-incubation with Ang-1 (FIG. 15A). When p190RhoGAP was inhibited, Ang-1 could no longer suppress endotoxin-mediated RhoA activation (FIG. 15B vs. FIG. 15A). This result identifies p190RhoGAP as a critical protein necessary for Ang-1 to block endotoxin-mediated RhoA activation.

Using immunofluorescence microscopy, we observed that 30 minutes of endotoxin exposure (100 ng/ml) resulted in dispersed junctional VE-cadherin and gap formation compared to vehicle-treated cells (FIG. 15C, panels a-b). Ang-1 reverted the endotoxin-induced derangements to a normal appearance (FIG. 15C, panel c). Delivery of Rac1T17N greatly diminished the ability of Ang-1 to “rescue” endotoxin-treated cells (FIG. 15C, panel d) as did inhibition of p190RhoGAP (FIG. 15C, panel e), resulting in persistent interendothelial gaps. Control virus or control siRNA had no effect on the response to endotoxin or endotoxin plus Ang-1. These results show that Ang-1 requires Rac1 and p190RhoGAP to reverse the structural derangements induced by endotoxin.

### Example 13

### Ang-1 Blocks Endotoxin-Induced Hyperpermeability Through PI3K, Rac1, and p190RhoGAP

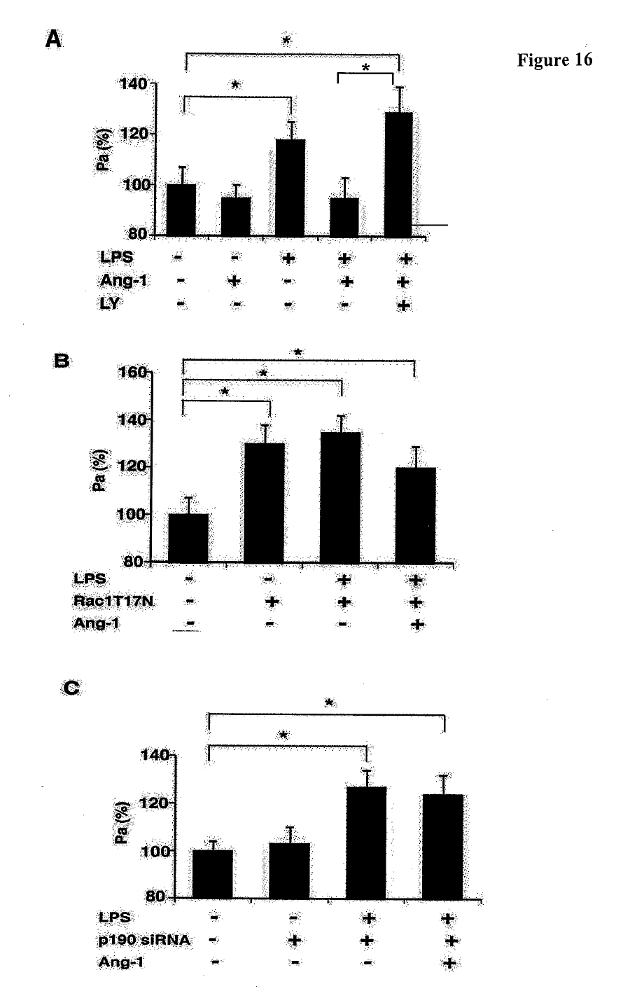

After observing that Ang-1 could reverse the endothelial structural changes induced by endotoxin, we next tested the effects of these ligands on permeability using a standard in vitro assay to quantify the flux of fluorescently-labeled albumin across a HMVEC-L monolayer grown to confluency. Ang-1 somewhat decreased basal monolayer permeability. Endotoxin increased permeability approximately 20%, an effect completely reversed by addition of Ang-1 (FIG. 16A). The protective effect of Ang-1 was lost when LY294002 was added (FIG. 16A). In the presence of Rac1T17N, basal permeability was increased, endotoxin failed to augment trans-monolayer leak further, and Ang-1 failed to reverse the hyperpermeability (FIG. 16B). p190RhoGAP siRNA had little effect on basal permeability, but did prevent the rescue effect of Ang-1 (FIG. 16C).

These results demonstrate that active PI3K, Rac1, and p190RhoGAP are necessary for Ang-1 to block the permeability effect of endotoxin across endothelial cells, much as they are necessary for Ang-1 to block the endothelial structural derangements induced by endotoxin (FIG. 15C). Rac1 and p190RhoGAP differ in that inhibition of the former is sufficient to produce hyperpermeability whereas inhibition of the latter is insufficient to produce leak across a monolayer.

### Example 14

### In Vivo Inhibition of p190RhoGAP Abolishes the Protective Effect of Systemic Ang-1 Against Endotoxemic Vascular Leak

Since p190RhoGAP is necessary for Ang-1 to inhibit several effects of endotoxin—RhoA activation (FIG. 15B), intercellular gap formation (FIG. 15C), and in vitro permeability (FIG. 16C)—we next addressed the importance of this pathway in vivo. Such validation is important because in vitro assays lack crucial elements such as unidirectional laminar flow, variable hydrostatic pressure, and interacting cell types (e.g., neutrophils and vascular smooth muscle cells), basement membrane, and matrix found in an animal model. Moreover, in vitro structural studies can readily demonstrate paracellular gaps whereas permeability in the context of a living organism may arise through a combination of transcellular and paracellular fluid movement (Mehta et al., Physiol. Rev. 86:279-367 (2006).

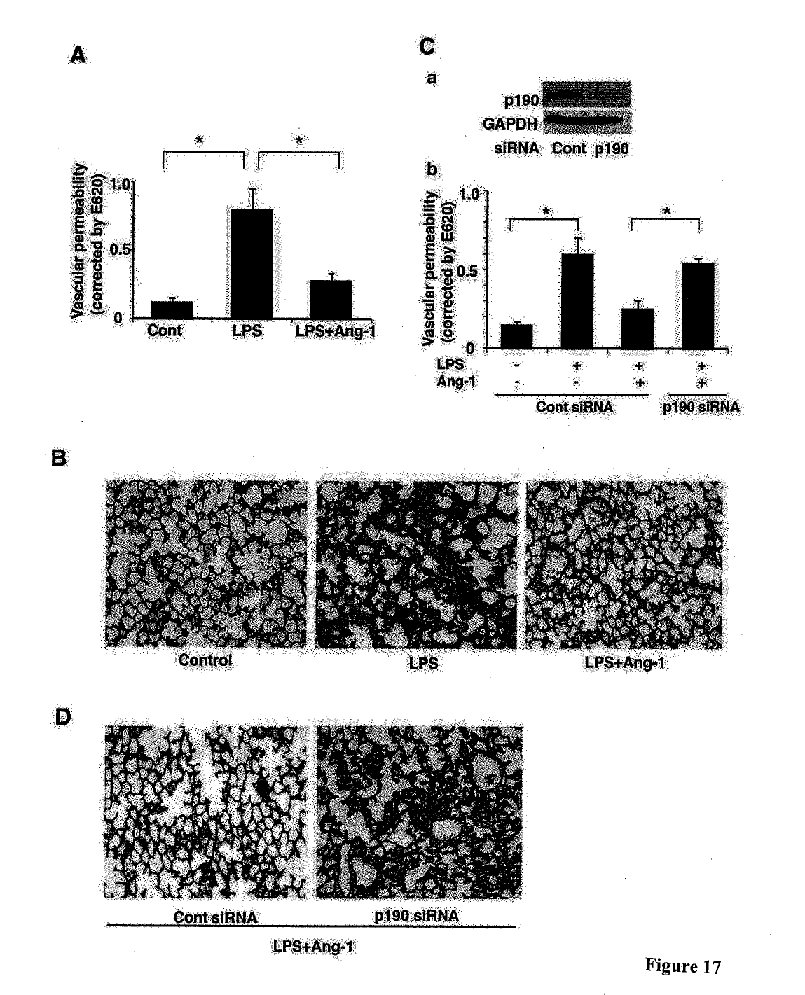

Evans blue dye avidly binds to serum albumin and can therefore be used as a tracer for flux of macromolecules across the microvasculature. In the low-permeability environment of the lung, we measured the effect of systemically administered endotoxin (100 mcg i.p.) and found an eight-fold increase in leakage of intravascular contents compared to controls (FIG. 17A). The increased permeability was blocked by Ang-1 (FIG. 17A). Light photomicrographs of lung sections revealed that systemic endotoxin resulted in interstitial edema and heavy infiltration of airspaces by leukocytes as compared to control lung sections and those taken from animals pre-treated with Ang-1 (FIG. 17B).

Delivery of p190RhoGAP siRNA, but not control siRNA, resulted in diminished p190RHoGAP expression in lung tissue (FIG. 17C, panel a). In mice receiving control siRNA, endotoxin administration increased lung vascular permeability, and this effect was rescued by Ang-1. Of note, p190RhoGAP siRNA was sufficient to block the protective effect of Ang-1 in the lung (FIG. 17C, panel b). Moreover, histological sections confirmed that p190RhoGAP knockdown diminished the ability of Ang-1 to block endotoxin-mediated interstitial edema and leukocyte infiltration (FIG. 17D). These results validate our earlier in vitro findings in the context of an otherwise healthy adult animal and extend them by firmly establishing the importance of p190RhoGAP as a critical intracellular toggle that determines the ability of Ang-1 to interrupt endotoxin-mediated permeability and inflammation.

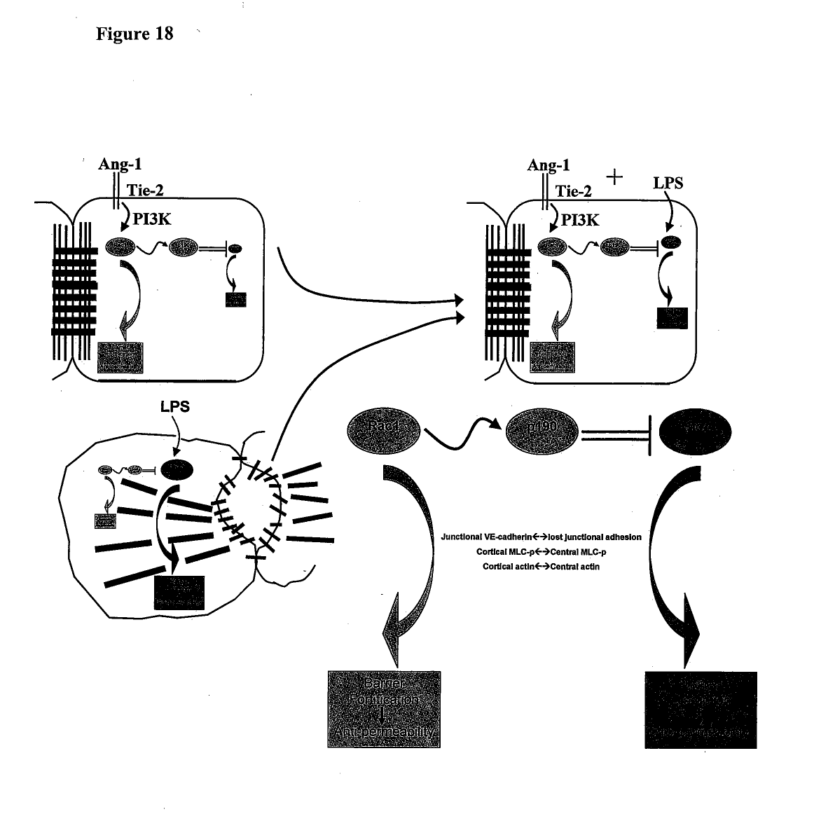

The data presented in Examples 10-14 demonstrate that Ang-1 protects against endotoxin-mediated vascular leakage by remodeling the endothelial cytoskeleton. To achieve this, Ang-1 signals through PI3K to activate Rac1, to activate p190RhoGAP, and to inhibit RhoA. A GTPase balance favoring Rac1 over RhoA promotes cell-cell adhesion and prevents the formation of intercellular gaps. Ang-1 is able to block the structural derangements and hyperpermeability induced by endotoxin, but requires both Rac1 and p190RhoGAP to do so. Rac1 and p190RhoGAP differ in that only inhibition of the former is sufficient to promote gap formation and increased permeability, but p190RhoGAP is necessary for Ang-1 to suppress endotoxin-mediated RhoA activity. As a result, inhibition of p190RhoGAP prevents Ang-1 from reversing the architectural derangements and hyperpermeability induced by endotoxin. To demonstrate its importance more conclusively, expression of p190RhoGAP was inhibited by in vivo siRNA. This manipulation abrogated the protection conferred by Ang-1 against endotoxin-mediated vascular leak and inflammation. A schematic summarizing the dichotomous actions of Ang-1 and endotoxin on Rac 1/RhoA balance is presented in FIG. 18.

Excess vascular leakiness plays a well-recognized and dramatic role in conditions such as sepsis and acute respiratory distress syndrome. Endotoxin has been implicated in the pathogenesis of both of these conditions. Our findings confirm and extend prior work that demonstrated a pro-survival effect of Ang-1 overexpression in mice subjected to endotoxemic shock (Witzenbichler et al., Circulation 111:97-105 (2005)). Examples 1-9, above, showed that the endogenous Tie-2 antagonist, Ang-2, is elevated in the circulation of human subjects with sepsis and pulmonary injury and that systemic Ang-2 administration to otherwise healthy adult mice is sufficient to promote severe vascular leak, most prominently in the lung (Parikh et al., PloS Med. 3:e46 (2006)). When considered together, the experiments described in these examples implicate Tie-2 as a critical transmembrane tyrosine kinase involved in the defense and maintenance of the vascular permeability barrier in common, life-threatening conditions.

A link between Rho-mediated endothelial cytoskeletal regulation and permeability was proposed almost ten years ago in studies using C3 bacterial exotoxin to inhibit RhoA (Hippenstiel et al., Am. J. Physiol Lung Cell Mol. Physiol. 272:L38-43 (1997), Garcia et al., Am. J. Physiol. Lung Cell Mol. Physiol. 276:989-998 (1999)). However, most investigations have focused on the effects of a single member of the Rho protein family on endothelial barrier function. Less well-characterized is the interaction of several Rho members to produce coordinated endothelial cytoskeletal responses that ultimately result in modulation of in vivo permeability. The contribution of Cdc42, the third major Rho family protein, remains to be clarified as one report suggests no effect on endothelial permeability (Wojciak-Stothard et al., J. Cell Sci. 114:1343-1355 (2001)), but a more recent study identifies a role for Cdc42 in junctional protein stabilization (Broman et al., Circ. Res. 98:73-80 (2006)).

Rac1 inhibition or p190RhoGAP inhibition negates the structural and functional protective effects of Ang-1 against endotoxin, but only inhibition of Rac1 activity is sufficient to derange the cytoskeleton and induce permeability. The fact that p190RhoGAP inhibition does not alter basal cytoskeletal structure or barrier function implies that de-suppression of RhoA is insufficient to remodel the cytoskeleton into a permeable phenotype. Rather, actual activation of RhoA, with a constitutively active RhoA or a RhoA stimulator such as endotoxin, is necessary to shift the basal cytoskeletal structure and barrier function of the microvascular endothelium. This is consistent with the regulatory role described for p190RhoGAP in other cell types (Arthur et al., Mol. Biol. Cell 12:2711-2720 (2001)). Therefore, p190RhoGAP appears to be dispensable for the endothelium at baseline, but crucial for the endothelial cell to defend against RhoA activation.

On the other hand, baseline Rac1 activity is necessary to maintain cell-cell adhesion and prevent excessive permeability. Rac1 may induce post-translational modification to stabilize VE-cadherin at cell junctions (Wojciak-Stothard et al., Am. J. Physiol. Lung Cell Mol. Physiol. 288:L749-760 (2001), Seebach et al., Thromb. Haemost. 94:620-629 (2005)). Conversely, junctional proteins may, in fact, activate Rac1 (so-called outside-in signaling) (Lampugnani et al., Mol. Biol. Cell 13:1175-1189 (2002)) to promote p190RhoGAP-mediated RhoA suppression, resulting in less centripetal tension and cell contraction (Holinstat et al., J. Biol. Chem. 281:2296-2305 (2006), Noren et al., J. Biol. Chem. 276:33305-33308 (2001)). The signaling between Rac1 and junctional proteins may help maintain the basal barrier function of the endothelium. Our results suggest that this basal system for maintaining barrier integrity is further augmented by the addition of a second positive regulator of Rac1, Ang-1, and broken by an independent stimulator of RhoA, endotoxin. Several other extracellular signals may also regulate endothelial permeability through Rho and/or Rac, such as thrombin (van Nieuw Amerongen et al., Circ. Res. 87:335-340 (2000)), sphingosine-1-phosphate (Garcia et al., J. Clin. Invest. 108:689-701 (2001)), lysophosphatidic acid (van Nieuw Amerongen et al., Vasc. Biol. 20:E127-133 (2000)), TGF-β (Clements et al., Am. J. Physiol. Lung Cell Mol. Physiol. 288:L294-306 (2005)), Ang-2 (Parikh et al., supra), and ligands of VCAM-1 and ICAM-1 (Laudanna et al., Science 271:981-983 (1996), Wojciak-Stothard et al., J. Cell Biol. 145:1293-1307 (1999)). Ample evidence, therefore, suggests that competing Rho GTPases may provide a downstream, conserved mechanism for regulation of vascular permeability by controlling EC shape and adhesion responses to diverse extracellular stimuli. Ang-1 may, therefore, counteract the destabilizing influence of multiple Rho activators.

Furthermore, additional proteins, including actin, myosin, and VE-cadherin, are necessary to execute the permeability effects signaled through Rho GTPases. For example, alpha-catenin may bridge the signaling pathway connecting Rac1 to VE-cadherin (Broman et al., Circ. Res. 98:73-80 (2006)), but this has not been tested in the setting of Ang-1 stimulation. Rho GTPases may also impact other aspects of EC behavior by controlling the cytoskeleton such as secretion of signaling molecules (Etienne et al., Nature 420:629-635 (2002)) and leukocyte adhesion (Thorlacius et al., J. Leukoc. Biol. 79:923-931 (2006)).

In this last respect, our in vivo results were also notable for the ability of Ang-1 to block endotoxin-induced infiltration of leukocytes into the lung parenchyma. Ang-1 downregulates expression of VCAM-1, ICAM-1, and E-selectin, thereby preventing initial leukocyte adhesion, so-called “leukocyte rolling” (Gamble et al., Circ. Res. 87:603-607 (2000), Kim et al., Circ. Res. 89:477-479 (2001)). Rolling leukocytes induce clustering of these adhesion proteins on the apical endothelial surface that leads to RhoA activation and results in interendothelial gaps through which leukocytes cross the endothelium (Millan et al., Biochem. J. 385:329-337 (2005)). Therefore, the anti-inflammatory nature of Ang-1 may arise both due to decreased adhesion molecule expression in ECs as well as suppression of clustering-induced RhoA activation. This latter effect may further augment the anti-permeability action of Ang-1 in vivo, by preventing a leukocyte-induced secondary increase in permeability.

To our knowledge, the results presented here are the first direct—siRNA rather than chemical inhibitor—demonstration of in vivo vascular permeability regulation by a Rho family protein. Even though systemic delivery of siRNA could reasonably be expected to affect multiple cell types, the nature of the ligands and model used for the rodent experiments enable us to focus on the endothelial effects of p190RhoGAP in vivo. Because Ang-1 acts on Tie-2, a receptor whose expression is limited to the endothelium, we infer that the anti-permeability effect against endotoxin is mediated at the level of the endothelial cell. Therefore, the simplest hypothesis to account for the effect of p190RhoGAP is that its expression within the pulmonary endothelium is the critical transducer of Ang-1 protection against lung vascular leak. Other possibilities remain—such as Ang-1 acting on non-endothelial cell types, or p190RhoGAP knockdown in another cell type indirectly attenuating the protective effect of Ang-1—but our in vitro signaling, structural, and functional data are in agreement with the hypothesis that endothelial p190RhoGAP is critical in vivo.

Our results provide a novel mechanism for the anti-permeability effect of Ang-1 in the vascular system and describe, in detail, competing effects on endothelial cytokeletal structure and cell-cell adhesions from two GTPase signaling pathways that ultimately regulate vascular permeability. Our work suggests that activation of endothelial p190RhoGAP is critical for Ang-1 to block endotoxin-induced vascular leak and inflammation in vivo. Lastly, this report affirms the importance of the endothelium in the defense against endotoxemic injury.

### Materials and Methods

The following materials and methods were used in the experiments described above.

### Chemicals:

Human recombinant Ang-1, CD14 and LPS-binding protein (LPB) were purchased from R&D systems (Minneapolis, Minn.). The PI3K inhibitor, LY294002 is from Cell Signaling Technology (Beverly, Mass.). Other reagents used in the experiments were obtained from Sigma (St Louis, Mo.).

### Cell Culture:

Human microvascular endothelial cells from lung (HMVEC-L) (Cambrex Bio Science Walkersville, Inc., Walkersville, Md.) were cultured in EBM-2 (Cambrex) supplemented with 5% fetal bovine serum (FBS) and growth factors according to the manufacturer's instructions. All stimulation experiments were performed after serum starvation which was performed by incubation in 0.25% FBS/EBM-2 for 24 hours. Our preliminary experiments showed that both soluble CD14 and LBP (LPS binding protein) were required for LPS signaling cascade in endothelial cells under serum starvation and we used a combination of these proteins for the in vitro experiments in the following concentrations: LPS (100 ng/ml), CD14 (100 ng/ml), LBP (10 ng/ml) combination.

### Western Blot Analysis:

HMVEC-L were washed with ice-cold PBS three times and lysed with ice-cold RIPA buffer (50 mM Tris-HCl pH7.4, 150 mM NaCl, 1% NP-40, 0.5% Sodium Deoxycholate, 0.1% SDS and 1 mM EDTA) supplemented with protease inhibitors (Roche Diagnostics, Indianapolis, Ind.), 1 mM NaF and 1 mM Na3VO4. Lysates were sonicated and centrifuged at 10,000 rpm for 10 minutes at 4° C., and supernatants were collected. Protein concentrations were determined by BCA protein assay with bovine serum albumin as standard (Pierce, Rockford, Ill.). Lysates were electrophoresed using NuPAGE system (Invitrogen Life Technologies, Franklin Lakes, N.J.) and transferred to PVDF membrane and immunoblotted with specific primary antibodies. Binding of primary antibodies was detected using horseradish peroxidase-conjugated secondary antibodies (Amersham Bioscience, Piscataway, N.J.) and SuperSignal WestDura (Pierce) as a chemiluminescence substrate. Primary antibodies were obtained from these suppliers: anti-phospho-Akt Ab (Ser 473), anti-Akt Ab and anti-phospho-myosin light chain 2 (Ser19) Ab were from Cell Signaling Technology; anti-GAPDH Ab was from Chemicon International (Temecula, Calif.).

### Immunoprecipitation:

We lysed HMVEC-L with Triton buffer (50 mM Tris-HCl pH 8.0, 150 mM NaCl, 1% Triton X 100, and 5 mM EDTA) supplemented with protease inhibitors (Roche Diagnostics), 1 mM NaF and 1 mM Na₃VO₄, adjusting protein concentration by BCA protein assay (Pierce) and incubated 200 μg of total protein with anti-Tie2-Ab (clone Ab33, Upstate, Lake Placid, N.Y.) or p190RhoGAP Ab (Transduction laboratory, Lexington, Ky.) for 3 hours, followed by incubation with protein A sepharose (Zymed, San Francisco, Calif.) for 2 hours at 4° C. After washing the beads, proteins were eluted by heating in SDS-sample buffer and detected by immunoblotting with anti-phospho-tyrosine (clone 4G10, Upstate), anti-Tie-2 Ab, or anti-p190RhoGAP Ab.

### PI3K Activity Assay:

After signal starvation, HMVEC-L were treated with vehicle or Ang-1 (100 ng/ml) for 15 minutes. Total and phosphorylated p85 subunits of PI3K were quantified using a commercial enzyme-linked immunosorbent assay according to the manufacturer's instruction (Active Motif, Carlsbad, Calif.). Phosphorylated PI3K p85 subunit was corrected by total PI3K.

### Rac1 and Rho Activity Assay:

These were performed and quantified using the commercially available kit according to the manufacturer's instruction (Cytoskeleton, Denver, Colo.). After signal starvation, HMVEC-L were treated with vehicle, Ang-1 (100 ng/ml) with or without inhibitor for the indicated time and harvested with lysis buffer. Following a brief centrifugation to remove cell debris, lysates from the cells containing equivalent protein concentrations were incubated for 60 minutes with 40 μL slurry of a GST-fusion protein composed of Rac1 or RhoA effector proteins coupled to agarose beads. After washing with lysis buffer, samples were subjected to immunoblotting and detected with anti-Rac1 or anti-RhoA antibody (Santa Cruz Biotechnology, Santa Cruz, Calif.). Whole cell lysates were also run to determine the total amount of Rac1 or RhoA protein.

### Immunohistochemistry:

HMVEC-L were grown to confluent on glass coverslips coated with 0.1% gelatin (Attachment factor, Cascade Biologics, Portland, Oreg.) in 5% FBS/EGM2. The cells were treated with reagents in 0.25% FBS EBM-2 for 30 minutes, then fixed for 20 minutes in 4% paraformaldehyde in PBS, incubated for 5 minutes in 0.3% Triton X-100 in PBS. After blocking, cells were stained with anti-phospho-MLC (Ser19) Ab or anti-VE-cadherin monoclonal Ab (BD Pharmingen, San Diego, Calif.) as first antibodies and with Alexa Fluoro® 488-conjugated secondary Abs (Molecular Probe, Eugene, Oreg.). We used rhodamine phalloidin (Molecular Probe) for F-actin staining and TOPRO®-3-iodine (Molecular Probe) for nuclear staining. Fluorescence images were obtained using a Bio Rad MRC confocal fluorescence microscope.

### Measurement of Endothelial Permeability In Vitro:

Coster Transwell membranes (Corning Inc. Corning, N.Y.) were coated with 0.5% Gelatin and cells were grown until confluence. Vehicle or proteins with FITC-albumin (1 mg/ml) was added to the luminal chamber for 4 hours, and samples were taken from both the luminal and abluminal chamber for fluorometry analysis. The readings were converted with the use of a standard curve to albumin concentration. These concentrations were then used in the following equation to determine the permeability coefficient of albumin (Pa).

$${P}_{a} = \frac{[ A ]}{t} \times \frac{1}{A} \times \frac{V}{[ L ]}$$

[A] is abluminal concentration; t is time in seconds; A is area of membrane in cm²; V is volume of abluminal chamber; and [L] is luminal concentration.
siRNA Transfection:
HMVEC-Ls were seeded and control small interfering RNA (siRNA) (Ambion, Ausin, Tex.) or siRNA directed to human p190RhoGAP (5′-GGAUUGUGUGGAAUGUAAG-3′ SEQ ID NO: 5 and 5′-CUUACAUUCCACACAAUCC-3′ SEQ ID NO: 6) was transfected using SilentFect Lipid reagent (Bio-Rad) according to the manufacturer's instructions. The cells were used for each experiment 3 days after transfection. Almost 90-100% cells were transfected with siRNA (checked by fluorescent labeled siRNA). Down-regulation of p190RhoGAP was verified by immunoblotting. We tested two different siRNA for the experiment and obtained similar results.

### Lentivirus Construction and Induction:

The dominant negative form of Rac1 (Rac1T17N) and the constitutively active form of RhoA (RhoAG14V) were constructed by PCR using pcDNA-Rac1T17N or -RhoAG14V (University of Missouri-Rolla cDNA Resource Center) as a template and subcloned into the pHAGE lentiviral backbone vector at the NotI/BamHI sites. Generation of lentiviral vectors was accomplished by a five-plasmid transfection procedure (Mostoslaysky et al., Mol. Ther. 11:932-940 (2005)). Briefly, 293T cells were transfected using TransIT 293 (Minis, Madison, Wis., USA) according to the manufacturer's instructions with the backbone pHAGE vector together with four expression vectors encoding the packaging proteins Gag-pol, Rev, Tat, and the G protein of the vesicular stomatitis virus (VSV). Viral supernatants were collected starting 48 hours after transfection, for four consecutive times every 12 hours, pooled, and filtered through a 0.45 μm filter. Viral supernatants were then concentrated 100-fold by ultracentrifugation in a Beckman centrifuge, for 1.5 hours at 16500 rpm. Using these protocols, titers of 5×10⁸ to 1×10⁹/ml were achieved. HMVEC-L were incubated with viral stocks in the presence of 5 μg/ml polybrene (sigma) and 90-100% infection was achieved 3 days later (checked by HA staining, Supplement E).

### In Vivo Permeability Assay:

Mice (8-12 weeks old, female FVB strain) were pretreated with Ang-1 (10 mcg, ip). 8 hours after the first Ang-1 injection, the second dose of Ang-1 (10 mcg, ip) and LPS (100 μg, ip) were co-injected. Lung permeability was assessed 16 hours after the second injection. Mice were anesthetized with Avertin (2,2,2-Tribromoethanol) and 2% Evans blue (50 μl) was then injected into the retro-orbital sinus. Ten minutes after Evans blue injection, mice were sacrificed and perfused with PBS with 2 mM EDTA for 10 minutes through a cannula placed in the right ventricle. After the perfusion, the outflow from the inferior vena cava was observed to be clear, confirming that blood (and intravascular Evans blue) had been flushed out of the circulation. Organs were then harvested and homogenized in 1.5 nil formamide. Evans blue was extracted by incubating the samples at 70° C. for 24 hours, and the concentration of Evans blue was estimated by dual-wavelength spectrophotometer (620 and 740 nm). The following formula was used to correct optical densities (E) for contamination with heme pigments: E620 (corrected)=E620−(1.426XE740+0.030).

### Histology:

Mice were treated as above and lungs were harvested, fixed in 10% formalin, embedded in paraffin, sectioned, and stained with hematoxilin and eosin.
In Vivo Delivery of siRNA:
Delivery of siRNA into mice was performed by TransIT® Hydrodynamic delivery solution (Mires, Madision, Wis.) per the manufacturer's instruction. Mice were injected with either 10 μg control siRNA or 10 μg p190RhoGAP siRNA in 2 nil delivery solution injected into the tail vein over 7 seconds. Four days later, p190RhoGAP knockdown was confirmed by lysing organs in RIPA buffer and performing Western analysis as outlined above. In vivo permeability and histological examination were performed as described above.

### Statistical Analysis:

Results are reported as mean±SEM. Comparisons between continuous variables were performed using unpaired two-sided t-test.

### Example 15

### Ang-2 is a Potential Mediator of High Dose (HD) Interleukin 2 (IL-2) Induced Vascular Leak

HD IL-2 is an FDA approved treatment for patients with metastatic renal cell carcinoma and metastatic melanoma. The mechanism of action of this cytokine based therapy is poorly understood and thought to depend on T cells and NK cell anti-tumor activity. Although only 10%-15% of those treated will show tumor response, the duration of effect in responders can reach ten years. HD IL-2 is the only available therapy that can offer such results.

Unfortunately, as many as 65% of patients receiving HD IL-2 will have interruption of therapy or discontinuation of treatment due to vascular leak syndrome (VLS) (Bascon, Immunopharmacology 39:255-257 (1998), Baluna et al., Immunopharmacology 37:117-132 (1997)). VLS is characterized by marked vasopermeability with hypotension universally requiring intravenous fluids, and frequently, pharmacologic vasopressor support. Other manifestations of IL2 induced VLS include prerenal azotemia, metabolic acidosis, hyperbilirubinemia, and transaminits. VLS is associated with leakage of protein rich fluid into the interstitium leading to potential end organ compromise, of which pulmonary edema with respiratory distress is the most clinically concerning (Berthiaume et al., Am. J. Respir. Crit. Care Med. 152:329-335 (1995), Lee et al., J. Clin. Oncol. 7:7-20 (1989)). There are no approved therapies to prevent or treat VLS other than holding IL-2 doses and providing supportive care.

It is well known that IL-2 causes endothelial cell activation with loss of proper barrier function (Cotran et al., J. Immunol. 140:1883-1888 (1988)), Yi et al., Am. J. Pathol. 140:659-663 (1992)). This may require interaction of endothelial cells and specific circulating leukocyte populations, but detailed signaling has not been determined (Li et al., Inflammation 20:361-372 (1996), Ohkubo et al., Cancer Res. 51:1561-1563 (1991), Kotasek et al., Cancer Res. 48:5528-5532 (1988) Assier et al., J. Immunol. 172:7661-7668 (2004)). With little mechanistic data on the pathways of the endothelial dysfunction, further work on the biology of VLS is needed to identify key molecules and targets for novel therapies.

As described above, we have discovered that in human subjects with sepsis and adult respiratory distress syndrome (ARDS), circulating Ang-2 levels were elevated to twenty times that of normals (Parikh et al., supra). Like IL-2 induced VLS, sepsis is a syndrome characterized by profound hypotension and end organ injury due in part to endothelial barrier derangement and unchecked vascular leak.

VLS from HD IL-2, because of this clinical similarity to sepsis, is a compelling model of endothelial barrier dysfunction given the marked temporal relationship of vascular leak to IL-2 administration. The ability to study biomarkers before, during, and after infusion of IL-2 could reveal more about the pathogenesis of sepsis as well as the mechanism of IL-2 induced toxicity.

We hypothesized that VLS from HDIL2 may be associated with elevations in circulating Ang-2. In a pilot study, we collected serum prior to infusion of IL-2 and one day after completion of IL-2 therapy from three subjects to measure Ang-2. We found an average pretreatment Ang-2 level of 4 ng/ml and post treatment level of 25 ng/ml (p<0.0008). Based on these positive results, we then collected daily serum samples on 14 additional subjects receiving HDIL-2 (Table 2)/

TABLE 2
Baseline Characteristics

| Patient | Age | Gender | Diagnosis |
|---|---|---|---|
| 1 | 62 | F | Melanoma |
| 2 | 49 | M | Melanoma |
| 3 | 54 | F | Melanoma |
| 4 | 27 | F | Melanoma |
| 5 | 48 | M | Melanoma |
| 6 | 41 | M | Melanoma |
| 7 | 51 | M | RCC |
| 8 | 59 | M | RCC |
| 9 | 60 | F | RCC |
| 10 | 24 | M | Melanoma |
| 11 | 56 | M | Melanoma |
| 12 | 70 | M | RCC |
| 13 | 66 | M | Melanoma |
| 14 | 49 | F | Melanoma |

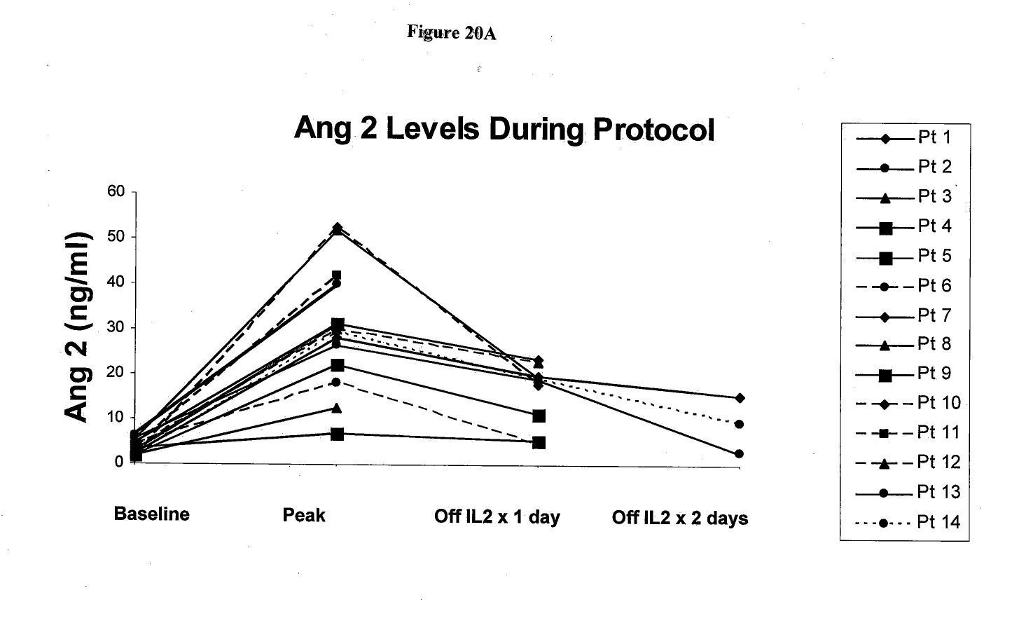

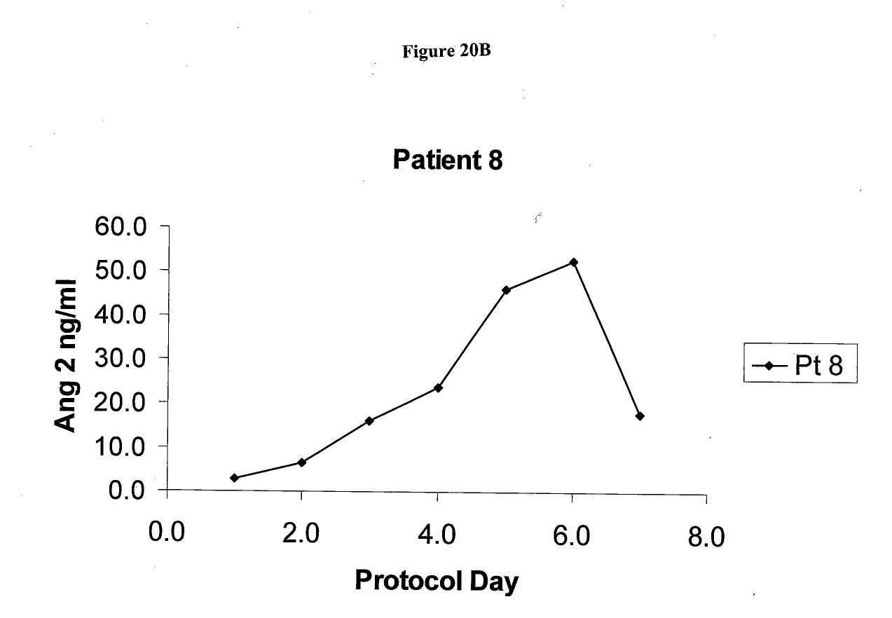

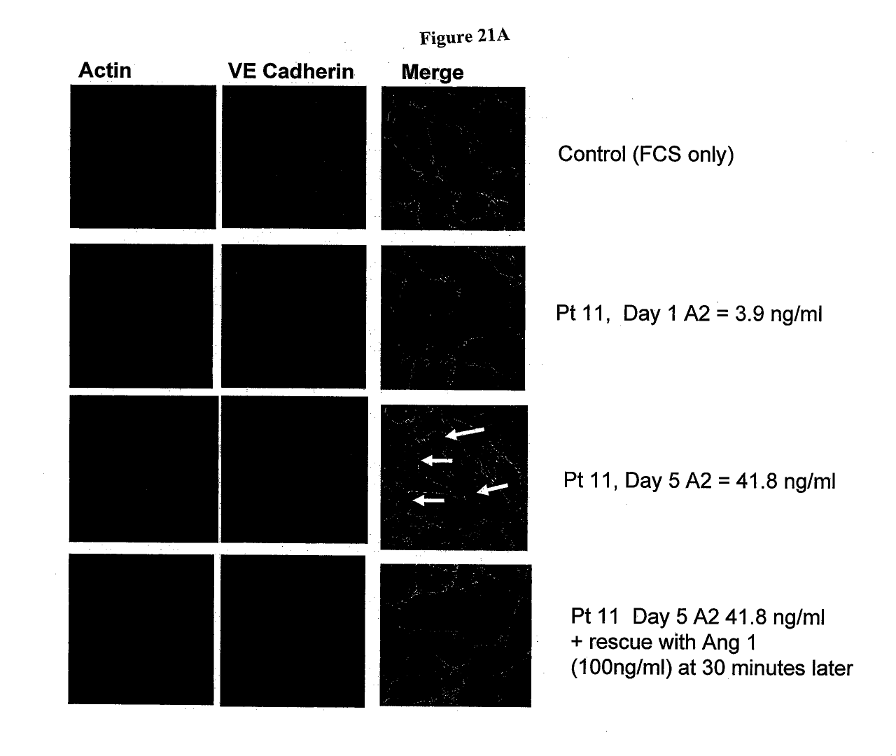

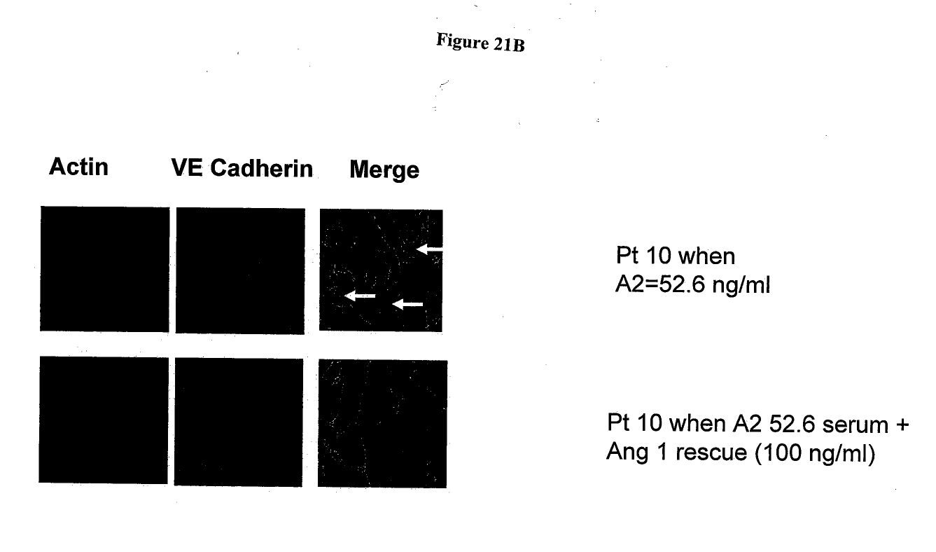

ELISA was used (as outlined in the Methods, below) to measure serum Ang-2. As shown in FIG. 20A-B, patients receiving HD IL-2 showed a steady rise in circulating Ang-2 throughout their infusion protocol and a decline after their final dose of HD IL-2. By protocol patients receive thrice daily infusions, but the final number of doses and doses per day are determined by institutional protocol designed to limit manifestations of VLS. Details on each patients HD IL-2 course is shown in Table 3.

TABLE 3
Patient Data

| | | | | % Weight |
|---|---|---|---|---|
| | | Baseline Ang | Peak Ang 2 | Gain from |
| Patient | # Doses IL2 | 2(ng/ml) | (ng/ml) | baseline (kgs) |
| 1 | 12 | 2.3 | 28.1 | 10.5 |
| 2 | 12 | 5.6 | 26.5 | 7.9 |
| 3 | 14 | 1.8 | 12.6 | 7.3 |
| 4 | 8 | 3.3 | 6.9 | 10.7 |
| 5 | 10 | 2.6 | 31.4 | 9.6 |
| 6 | 13 | 4.5 | 18.2 | 12.0 |
| 7 | 14 | 4.7 | 31.4 | 10.0 |
| 8 | 10 | 5.4 | 51.9 | 13.4 |
| 9 | 11 | 1.8 | 22.3 | 7.4 |
| 10 | 12 | 1.3 | 52.6 | 5.5 |
| 11 | 13 | 3.9 | 41.8 | 8.3 |
| 12 | 12 | 3.3 | 30.0 | 10.2 |
| 13 | 9 | 6.3 | 40.0 | 8.3 |
| 14 | 10 | 1.7 | 29.4 | 13.3 |

All patients had a significant rise of Ang-2 from baseline. All patients had hypotension requiring IVF boluses and median weight gain was 9.8% from admission. Five of the fourteen patients required vasopressors in addition to IVF and four required supplemental oxygen. Neither the peak Ang-2 level nor the fold rise in Ang-2 was correlated with a vasopressor or oxygen requirement.

The mechanism for Ang-2 inducing vasopermeability is believed to be through blockade of normal phosphorylation of the endothelial specific Tie 2 receptor. This leads to upregulation of RhoA, a protein in the GTPase family which in turn increases phosphorylation of the myosin light chain with resultant actin stress fiber formation (see examples above). Such cytoskeletal changes lead to cellular contraction with disruption of endothelial junctional proteins, such as vascular endothelial cadherins, and formation of inter-endothelial gaps and increased permeability as described above.

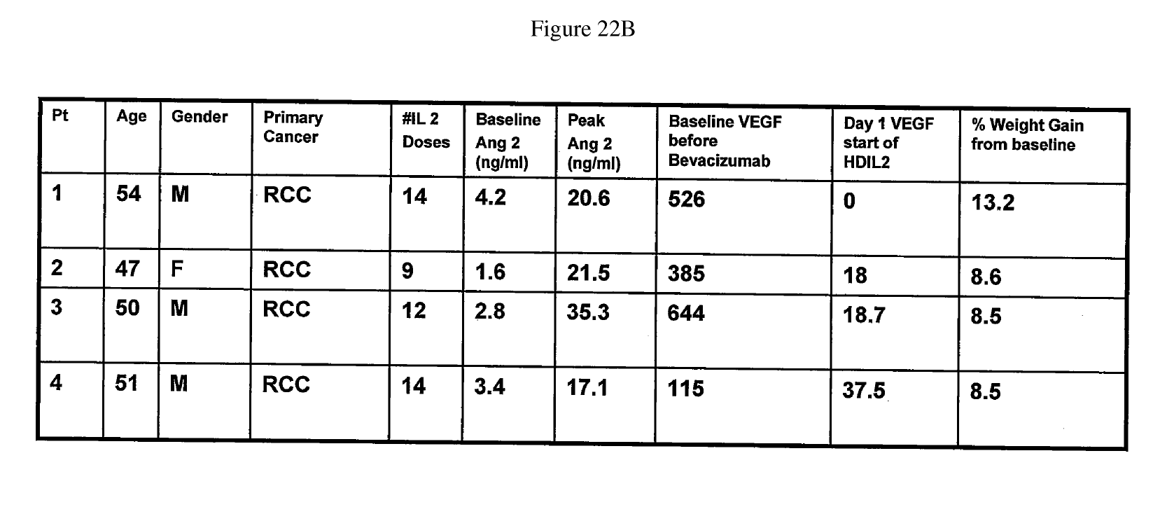

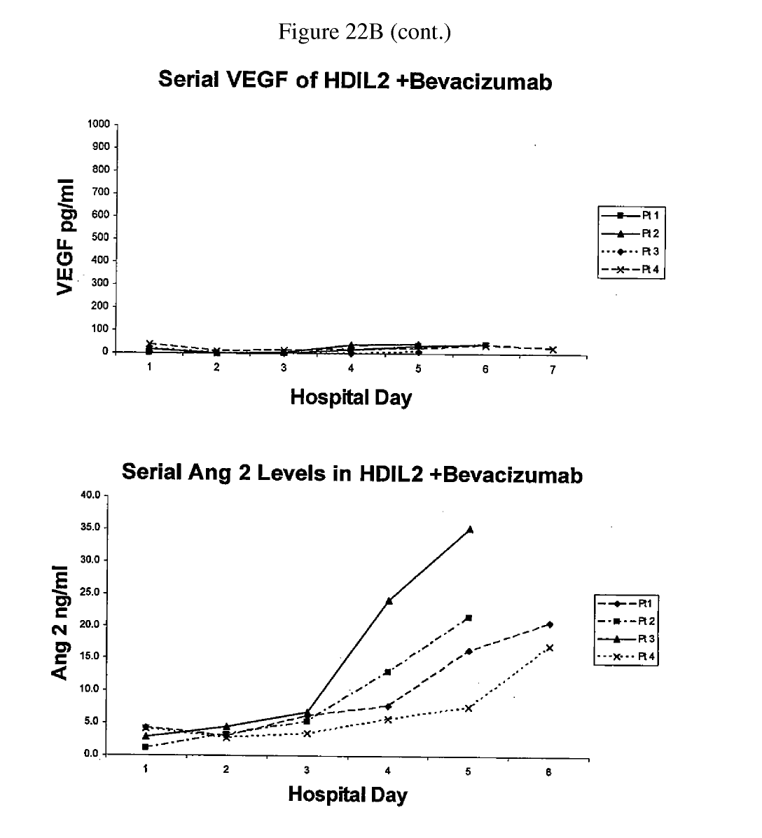

To determine the functional importance of elevated Ang 2 in HD IL-2 recipients developing VLS, we performed immunostaining experiments. Cultured monolayers of human pulmonary microvascular endothelial cells (HMVEC-L) were bathed in a 1:10 dilution of high Ang-2 patient serum and the effect on cell structure was examined. As outlined in the Methods, below, cells were fixed and stained for actin and for VE cadherin. As shown in FIG. 22A, when the low Ang 2 (3.9 ng/ml) patient sera was applied to confluent HMVEC-L monolayers, the phenotype was similar to control with minimal actin stress fiber formation and circumferential VE cadherin cell surface expression, suggesting intact cell-cell contacts. However, when that same patient's serum on HDIL2 infusion Day 5 high Ang-2 (41.8 ng/ml) was incubated with HMVEC-L monolayers, actin stress fiber formation and endothelial gap formation developed. Additionally, endothelial barrier integrity as represented by VE cadherin was disrupted. To determine whether this effect was mediated by Ang-2 specifically rather than other soluble molecules, HMVEC-1 monolayers were bathed with the high Ang-2 serum for 30 minutes and then treated with the endogenous Ang-2 antagonist Ang-1. Under these conditions, there was a significant attenuation in stress fiber formation and reforming of contiguous VE cadherin cell junction expression. FIG. 22B shows that this rescue effect of Ang-1 was confirmed in another patient with peak Ang-2 level of 52.6 ng/ml.

These results demonstrate that rising Ang-2 correlates with development of VLS; falling Ang-2 correlates with cessation of HD IL-2 and recovery from VLS, and Ang-2 in patient sera is sufficient to induce endothelial cell disruption.

We next asked whether VEGF, a canonical vascular leak factor, may be downstream of Ang-2. The possibility of VEGF being the underlying driving force in HDIL2 VLS is further supported by the fact that patients with metastatic renal cell and melanoma have higher circulating VEGF levels compared to normals and that treatment with HDIL2 can cause further VEGF elevation (Negrier et al., J. Clin. Oncol. 22:2371-2378 (2004)). To assess for a potential confounding effect of VEGF in HD IL-2 VLS, we first measured serial free VEGF levels in 8 subjects on HD IL-2 (FIG. 22A). Though baseline levels were elevated compared to normal subjects, there was no trend seen during HDIL2 infusion.

We then studied a unique patient population at our medical center. Four patients were placed on a new protocol in which they pretreated with a single infusion of the anti-VEGF therapy, bevacizumab, two weeks prior to receiving HD IL-2. This would provide a population with near zero levels of free VEGF at time of HD IL-2 therapy and should eliminate the question of whether VEGF is a confounding factor in HD IL-2 VLS. As shown in FIG. 22B, all bevacizumab plus HD IL-2 patient developed VLS with hypotension and weight gain. The free VEGF levels were low throughout protocol, but the Ang-2 rose without a blunting of the rise in slope or peak levels compared to HDIL2 alone treated patients.

### Discussion

High-dose bolus IL2 is a potentially curative treatment for 10-15% of patients with metastatic renal cell carcinoma and melanoma, but with considerable toxicity and cost. Over the past year, two new targeted therapies, sorafenib (Bayer-Onyx) and sunitinib (Pfizer) have been approved for the treatment of RCC. While these agents expand the repertoire of options for patients with RCC, both are given indefinitely, are associated with toxicities, and lead to few complete responses. Moreover, resistance to these agents develops in about 8-12 months, leaving room for more treatment options, either as salvage or first line, prior to these newer therapies. In contrast to RCC, patients with metastatic melanoma have more limited treatment options. Thus, in this patient population, improving the toxicity profile of HD IL-2 would be a valuable therapeutic advance.

VLS is a life-threatening toxicity of HD IL-2 therapy for which there is little mechanistic understanding and no specific therapy, other than supportive care. If VLS could be avoided or decreased more patients could safely receive this treatment option and have a chance at a durable tumor response.

The previous examples report elevated levels of Ang-2 in the serum of patients with septic shock, and because of the striking clinical similarities in sepsis and HDIL2 VLS, we hypothesized that Ang-2 could also mediate vascular leak in subjects on this biologic therapy.

Ang 2 levels rose dramatically in 14/14 patients with HD IL-2 administration and continued to rise with ongoing IL-2 exposure. Cessation of HD IL-2 resulted in resolution of VLS and fall in circulating Ang-2. All patients treated with IL-2 exhibited signs and symptoms of VLS as measured by hypotension and weight gain. A subset showed more severe vascular leak as manifested by an oxygen requirement due to pulmonary edema, and/or hypotension requiring vasopressor support. There was no correlation between the peak Ang-2 levels or the fold increase and severe VLS endpoints of oxygen or vasopressor requirement. All subjects had a clear rise in Ang-2 but, due to the relatively small number of patients, it may be difficult to correlate absolute Ang-2 values with our endpoints. We did not have access to more detailed data such as continuous blood pressure monitoring, oxygen saturations, serum lactates, or even PaO2 levels which could be more sensitive in quantifying degree of VLS. Detailed information of that nature could further elucidate a quantitative temporal relationship between absolute Ang 2 levels and manifestations of VLS.

In seeking a mechanistic explanation for high Ang-2 correlating with VLS, we were able to demonstrate that Ang-2 is the likely endothelial disrupting factor in HD IL-2 patient serum by rescuing its effect on HMVEC-1's with specific Ang-2 antagonism. Because VEGF has been described as a mediator of vascular leak we measured serial VEGF levels in patients treated with HD IL-2. Though baseline levels are elevated above normals, no trends pre, post, or during therapy were noted. To further explore the role of VEGF in IL-2 induced vascular leak, we capitalized on a subset of patients treated with IL-2 and bevacizumab. Interestingly, despite having absent or low free VEGF levels, patients treated with IL-2 and bevacizumab, had elevated Ang-2 levels and still experienced VLS. This finding indicates that Ang-2 induction may be responsible for the VLS in these patients and possibly even in patients treated with HD IL-2 but without bevacizumab.

Together, this data suggests that an Ang-2 inhibitor could be a useful therapeutic for attenuating VLS in HD IL-2 therapy. Furthermore, the use of an Ang-2 inhibitor in a clinical setting as for HD IL-2 therapy would address the major dose limiting side effect of HD IL-2 protocols and potentially allow for increased IL-2 dosing with increased response rate. An additional reason to consider Ang-2 antagonism in this population, is found in work by Oliner et al., supra, who showed that selective blockade of Ang-2 inhibited tumor angiogenesis, and decreased tumor burden in murine models of colon cancer (Oliner et al., Cancer Cell 6:507-516 (2004)). This secondary benefit further underscores the potential clinical value of an Ang-2 antagonist in attenuating HD IL-2 therapy. Antibodies that specifically neutralize the activity of Ang-2 would be good candidates to be tested in patients receiving HD IL-2.

### Materials and Methods

The following materials and methods were used for the experiments described in Example 15.

### Samples:

Over a 6 month period at Beth Israel Deaconess Medical Center we prospectively collected discarded serum and plasma samples from oncology patients receiving high dose IL-2. In our first feasibility protocol on 4 patients, we collected only Day 1—before HD IL-2 and Day 6 (after HD IL-2). After detailing these promising results, we then collected bloods before IL-2 infusion and with each successive days morning labs. Whenever possible, we also obtained discarded blood from all follow up outpatient appointments. All identifying information was removed and data was encoded to protect patient privacy. Clinical data was collected on each patient by chart review. This study was approved by the institutional IRB.

Of note, four patients received an anti-VEGF agent bevacizumab two weeks before starting HD IL-2 and we also collected baseline, bevacizumab treated, and HDIL2 protocol samples on these patients.

### ELISA:

Ang-2 was measured in serum samples from patients by sandwich ELISA using the reagents and protocol supplied with the human Ang-2 ELISA kit (R&D Systems, Minneapolis, Minn., United States). Preliminary experiments confirmed the stability of Ang-2 in serum for 6-12 hours at room temperature as well as its stability through several freeze-thaw cycles.

### Cell Culture:

Human pulmonary arterial microvascular endothelial cells (HMVECs) (Cambrex Bio Science, Walkersville, Md., United States) cells were cultured in EBM-2 (Cambrex Bio Science) supplemented with 5% fetal bovine serum (FBS) and growth factors according to the manufacturer's instructions. Serum starvation was performed by incubation in 0.25% FBS/EBM-2 for 24 hours.

### Immunofluorescence:

HMVECs were grown to confluence on glass coverslips coated with 1% gelatin. The cells were fixed for 10 minutes in 4% paraformaldehyde in PBS, and incubated for 5 min in 0.5% Triton X-100 in PBS. After blocking, the monolayers were processed for staining with anti-VE-cadherin monoclonal antibody (BD Biosciences Pharmingen, San Diego, Calif., United States) and Alexa Fluor® 488 goat anti-mouse IgG, rhodamine phalloidin (Molecular Probes, Eugene, Oreg., California) for F-actin staining and TOPRO-3-iodine (Molecular Probes) for nuclear staining. Fluorescence images were obtained using a Bio Rad MRC confocal fluorescence microscope. For experiments using cells treated with serum from patients, serum Ang-2 concentration was first measured by ELISA. Then, patient serum was diluted to 10% with EBM-2 and filtered with low-protein-binding PVL) F membrane (0.22 lm Millipore) prior to application on EC monolayers.

### Other Embodiments

From the foregoing description, it will be apparent that variations and modifications may be made to the invention described herein to adopt it to various usages and conditions. Such embodiments are also within the scope of the following claims.

All publications, patent applications, and patents mentioned in this specification, including U.S. provisional application Nos. 60/798,639 and 60/716,339, are herein incorporated by reference to the same extent as if each independent publication, patent application, or patent was specifically and individually indicated to be incorporated by reference.

From the foregoing description, one skilled in the art can easily ascertain the essential characteristics of this invention; can make various changes and modifications of the invention to adapt it to various usages and conditions. Thus, other embodiments are also within the claims.

What is claimed is:

1. A method of diagnosing a subject as having, or having a risk of developing, a vascular leak, said method comprising measuring the level of an Ang-2 nucleic acid molecule in a sample from said subject and comparing it to a reference, wherein an alteration in said levels compared to a reference is a diagnostic indicator of a vascular leak, or a risk of developing a vascular leak.

2. The method of claim 1, wherein said subject has a vascular leak disorder selected from the group consisting of suffering from sepsis; pneumonia; ALI; ARDS; idiopathic capillary leak syndrome; vascular leak associated with high dose IL-2 therapy or rituximab therapy; pre-eclampsia; eclampsia; hypotensive states due to sepsis; heart failure; trauma; infection; pulmonary aspiration of stomach contents; pulmonary aspiration of water; near drowning; burns; inhalation of noxious fumes; fat embolism; blood transfusion; amniotic fluid embolism; air embolism; edema; organ failure; poisoning; radiation; acute and chronic vascular rejection; pancreatitis; trauma; vasculitis; C1 esterase inhibitor deficiency; TNF receptor associated periodic fever syndrome; massive blood transfusion; anaphylaxis; post-lung or post-heart-lung transplant; and ovarian hyperstimulation syndrome.

3. The method of claim 2, wherein said subject has a vascular leak disorder associated with high dose IL-2 therapy.

4. The method of claim 1, wherein said alteration in said levels is an increase.

5. The method of claim 1, wherein said measuring of levels is done on two or more occasions and a change in said levels between measurements is a diagnostic indicator of said vascular leak.

6. The method of claim 1, wherein said sample is a bodily fluid, cell, or tissue sample from said subject in which said Ang-2 is normally detectable.

7. The method of claim 6, wherein said bodily fluid is selected from the group consisting of urine, blood, serum, plasma, and cerebrospinal fluid.

8. The method of claim 2, wherein said subject is a human.

9. A method of diagnosing a subject as having, or a risk of developing a vascular leak, said method comprising determining the nucleic acid sequence of an Ang-2 gene in a sample from a subject and comparing it to a reference sequence, wherein an alteration in the subject's Ang-2 nucleic acid sequence that is an alteration that changes the expression level of the gene product in said subject diagnoses the subject with a vascular leak or a risk of developing a vascular leak.

10. The method of claim 9, wherein said sample is a bodily fluid, cell, or tissue sample from said subject in which said Ang-2 is normally detectable.

11. The method of claim 10, wherein said bodily fluid is selected from the group consisting of urine, blood, serum, plasma, and cerebrospinal fluid.

12. The method of claim 9, wherein said subject is a human.


SEQUENCE LISTING (6 sequences)

SEQ ID NO: 1 - protein, 496 aa, Homo sapiens
MWQIVFFTLSCDLVLAAAYNNFRKSMDSIGKKQYQVQHGSCSYTFLLPEMDNCRSSSSPYVSNAVQRDAPLEYDDSVQRLQVLENIMENNTQWLMKLENYIQDNMKKEMVEIQQNAVQNQTAVMIEIGTNLLNQTAEQTRKLTDVEAQVLNQTTRLELQLLEHSLSTNKLEKQILDQTSEINKLQDKNSFLEKKVLAMEDKHIIQLQSIKEEKDQLQVLVSKQNSIIEELEKKIVTATVNNSVLQKQQHDLMETVNNLLTMMSTSNSAKDPTVAKEEQISFRDCAEVFKSGHTTNGIYTLTFPNSTEEIKAYCDMEAGGGGWTIIQRREDGSVDFQRTWKEYKVGFGNPSGEYWLGNEFVSQLTNQQRYVLKIHLKDWEGNEAYSLYEHFYLSSEELNYRIHLKGLTGTAGKISSISQPGNDFSTKDGDNDKCICKCSQMLTGGWWFDACGPSNLNGMYYPQRQNTNKFNGIKWYYWKGSGYSLKATTMMIRPADF

SEQ ID NO: 2 - DNA, 2269 bp, Homo sapiens
tgggttggtgtttatctcctcccagccttgagggagggaacaacactgtaggatctggggagagaggaacaaaggaccgtgaaagctgctctgtaaaagctgacacagccctcccaagtgagcaggactgttcttcccactgcaatctgacagtttactgcatgcctggagagaacacagcagtaaaaaccaggtttgctactggaaaaagaggaaagagaagactttcattgacggacccagccatggcagcgtagcagccctgcgtttcagacggcagcagctcgggactctggacgtgtgtttgccctcaagtttgctaagctgctggtttattactgaagaaagaatgtggcagattgttttctttactctgagctgtgatcttgtcttggccgcagcctataacaactttcggaagagcatggacagcataggaaagaagcaatatcaggtccagcatgggtcctgcagctacactttcctcctgccagagatggacaactgccgctcttcctccagcccctacgtgtccaatgctgtgcagagggacgcgccgctcgaatacgatgactcggtgcagaggctgcaagtgctggagaacatcatggaaaacaacactcagtggctaatgaagcttgagaattatatccaggacaacatgaagaaagaaatggtagagatacagcagaatgcagtacagaaccagacggctgtgatgatagaaatagggacaaacctgttgaaccaaacagctgagcaaacgcggaagttaactgatgtggaagcccaagtattaaatcagaccacgagacttgaacttcagctcttggaacactccctctcgacaaacaaattggaaaaacagattttggaccagaccagtgaaataaacaaattgcaagataagaacagtttcctagaaaagaaggtgctagctatggaagacaagcacatcatccaactacagtcaataaaagaagagaaagatcagctacaggtgttagtatccaagcaaaattccatcattgaagaactagaaaaaaaaatagtgactgccacggtgaataattcagttcttcaaaagcagcaacatgatctcatggagacagttaataacttactgactatgatgtccacatcaaactcagctaaggaccccactgttgctaaagaagaacaaatcagcttcagagactgtgctgaagtattcaaatcaggacacaccacaaatggcatctacacgttaacattccctaattctacagaagagatcaaggcctactgtgacatggaagctggaggaggcgggtggacaattattcagcgacgtgaggatggcagcgttgattttcagaggacttggaaagaatataaagtgggatttggtaacccttcaggagaatattggctgggaaatgagtttgtttcgcaactgactaatcagcaacgctatgtgcttaaaatacaccttaaagactgggaagggaatgaggcttactcattgtatgaacatttctatctctcaagtgaagaactcaattataggattcaccttaaaggacttacagggacagccggcaaaataagcagcatcagccaaccaggaaatgattttagcacaaaggatggagacaacgacaaatgtatttgcaaatgttcacaaatgctaacaggaggctggtggtttgatgcatgtggtccttccaacttgaacggaatgtactatccacagaggcagaacacaaataagttcaacggcattaaatggtactactggaaaggctcaggctattcgctcaaggccacaaccatgatgatccgaccagcagatttctaaacatcccagtccacctgaggaactgtctcgaactattttcaaagacttaagcccagtgcactgaaagtcacggctgcgcactgtgtcctcttccaccacagagggcgtgtgctcggtgctgacgggacccacatgctccagattagagcctgtaaactttatcacttaaacttgcatcacttaacggaccaaagcaagaccctaaacatccataattgtgattagacagaacacctatgcaaagatgaacccgaggctgagaatcagactgacagtttacagacgctgctgtcacaaccaagaatgttatgtgcaagtttatcagtaaataactggaaaacagaacacttatgttatacaatacagatcatcttggaactgcattcttctgagcactgtttatacactgtgtaaatacccatatgtcct

SEQ ID NO: 3 - protein, 498 aa, Homo sapiens
MTVFLSFAFLAAILTHIGCSNQRRSPENSGRRYNRIQHGQCAYTFILPEHDGNCRESTTDQYNTNALQRDAPHVEPDFSSQKLQHLEHVMENYTQWLQKLENYIVENMKSEMAQIQQNAVQNHTATMLEIGTSLLSQTAEQTRKLTDVETQVLNQTSRLEIQLLENSLSTYKLEKQLLQQTNEILKIHEKNSLLEHKILEMEGKHKEELDTLKEEKENLQGLVTRQTYIIQELEKQLNRATTNNSVLQKQQLELMDTVHNLVNLCTKEGVLLKGGKREEEKPFRDCADVYQAGFNKSGIYTIYINNMPEPKKVFCNMDVNGGGWTVIQHREDGSLDFQRGWKEYKMGFGNPSGEYWLGNEFIFAITSQRQYMLRIELMDWEGNRAYSQYDRFHIGNEKQNYRLYLKGHTGTAGKQSSLILHGADFSTKDADNDNCMCKCALMLTGGWWFDACGPSNLNGMFYTAGQNHGKLNGIKWHYFKGPSYSLRSTTMMIRPLDF

SEQ ID NO: 4 - DNA, 4338 bp, Homo sapiens
ggggcacactcatgcattcctgtcaagtcatcttgtgaaaggctgcctgcttccagcttggcttggatgtgcaaccttaataaaactcactgaggtctgggagaaaatagcagatctgcagcagatagggtagaggaaagggtctagaatatgtacacgcagctgactcaggcaggctccatgctgaacggtcacacagagaggaaacaataaatctcagctactatgcaataaatatctcaagttttaacgaagaaaaacatcattgcagtgaaataaaaaattttaaaattttagaacaaagctaacaaatggctagttttctatgattcttcttcaaacgctttctttgagggggaaagagtcaaacaaacaagcagttttacctgaaataaagaactagttttagaggtcagaagaaaggagcaagttttgcgagaggcacggaaggagtgtgctggcagtacaatgacagttttcctttcctttgctttcctcgctgccattctgactcacatagggtgcagcaatcagcgccgaagtccagaaaacagtgggagaagatataaccggattcaacatgggcaatgtgcctacactttcattcttccagaacacgatggcaactgtcgtgagagtacgacagaccagtacaacacaaacgctctgcagagagatgctccacacgtggaaccggatttctcttcccagaaacttcaacatctggaacatgtgatggaaaattatactcagtggctgcaaaaacttgagaattacattgtggaaaacatgaagtcggagatggcccagatacagcagaatgcagttcagaaccacacggctaccatgctggagataggaaccagcctcctctctcagactgcagagcagaccagaaagctgacagatgttgagacccaggtactaaatcaaacttctcgacttgagatacagctgctggagaattcattatccacctacaagctagagaagcaacttcttcaacagacaaatgaaatcttgaagatccatgaaaaaaacagtttattagaacataaaatcttagaaatggaaggaaaacacaaggaagagttggacaccttaaaggaagagaaagagaaccttcaaggcttggttactcgtcaaacatatataatccaggagctggaaaagcaattaaacagagctaccaccaacaacagtgtccttcagaagcagcaactggagctgatggacacagtccacaaccttgtcaatctttgcactaaagaaggtgttttactaaagggaggaaaaagagaggaagagaaaccatttagagactgtgcagatgtatatcaagctggttttaataaaagtggaatctacactatttatattaataatatgccagaacccaaaaaggtgttttgcaatatggatgtcaatgggggaggttggactgtaatacaacatcgtgaagatggaagtctagatttccaaagaggctggaaggaatataaaatgggttttggaaatccctccggtgaatattggctggggaatgagtttatttttgccattaccagtcagaggcagtacatgctaagaattgagttaatggactgggaagggaaccgagcctattcacagtatgacagattccacataggaaatgaaaagcaaaactataggttgtatttaaaaggtcacactgggacagcaggaaaacagagcagcctgatcttacacggtgctgatttcagcactaaagatgctgataatgacaactgtatgtgcaaatgtgccctcatgttaacaggaggatggtggtttgatgcttgtggcccctccaatctaaatggaatgttctatactgcgggacaaaaccatggaaaactgaatgggataaagtggcactacttcaaagggcccagttactccttacgttccacaactatgatgattcgacctttagatttttgaaagcgcaatgtcagaagcgattatgaaagcaacaaagaaatccggagaagctgccaggtgagaaactgtttgaaaacttcagaagcaaacaatattgtctcccttccagcaataagtggtagttatgtgaagtcaccaaggttcttgaccgtgaatctggagccgtttgagttcacaagagtctctacttggggtgacagtgctcacgtggctcgactatagaaaactccactgactgtcgggctttaaaaagggaagaaactgctgagcttgctgtgcttcaaactactactggaccttattttggaactatggtagccagatgataaatatggttaatttcatgtaaaacagaaaaaaagagtgaaaaagagaatatacatgaagaatagaaacaagcctgccataatcctttggaaaagatgtattataccagtgaaaaggtgttatatctatgcaaacctactaacaaattatactgttgcacaattttgataaaaatttagaacagcattgtcctctgagttggttaaatgttaatggatttcagaagcctaattccagtatcatacttactagttgatttctgcttacccatcttcaaatgaaaattccatttttgtaagccataatgaactgtagtacatggacaataagtgtgtggtagaaacaaactccattactctgatttttgatacagttttcagaaaaagaaatgaacataatcaagtaaggatgtatgtggtgaaaacttaccacccccatactatggttttcatttactctaaaaactgattgaatgatatataaatatatttatagcctgagtaaagttaaaagaatgtaaaatatatcatcaagttcttaaaataatatacatgcatttaatatttcctttgatattatacaggaaagcaatattttggagtatgttaagttgaagtaaaagcaagtactctggagcagttcattttacagtatctacttgcatgtgtatacatacatgtaacttcattattttaaaaatatttttagaactccaatactcaccctgttatgtcttgctaatttaaattttgctaattaactgaaacatgcttaccagattcacactgttccagtgtctataaaagaaacactttgaagtctataaaaaataaaataattataaatatcattgtacatagcatgtttatatctgcaaaaaacctaatagctaattaatctggaatatgcaacattgtccttaattgatgcaaataacacaaatgctcaaagaaatctactatatcccttaatgaaatacatcattcttcatatatttctccttcagtccattcccttaggcaatttttaatttttaaaaattattatcaggggagaaaaattggcaaaactattatatgtaagggaaatatatacaaaaagaaaattaatcatagtcacctgactaagaaattctgactgctagttgccataaataactcaatggaaatattcctatgggataatgtattttaagtgaatttttggggtgcttgaagttactgcattattttatcaagaagtcttctctgcctgtaagtgtccaaggttatgacagtaaacagtttttattaaaacatgagtcactatgggatgagaaaattgaaataaagctactgggcctcctctcataaaagagacagttgttggcaaggtagcaataccagtttcaaacttggtgacttgatccactatgccttaatggtttcctccatttgagaaaataaagctattcacattgttaagaaaaatactttttaaagtttaccatcaagtcttttttatatttatgtgtctgtattctacccctttttgccttacaagtgatatttgcaggtattataccatttttctattcttggtggcttcttcatagcaggtaagcctctccttctaaaaacttctcaactgttttcatttaagggaaagaaaatgagtattttgtccttttgtgttcctacagacactttcttaaaccagtttttggataaagaatactatttccaaactcatattacaaaaacaaaataaaataataaaaaaagaaagcatgatatttactgttttgttgtctgggtttgagaaatgaaatattgtttccaattatttataataaatcagtataaaatgttttatgattgttatgtgtattatgtaatacgtacatgtttatggcaatttaacatgtgtattcttttaattgtttcagaataggataattaggtattcgaattttgtctttaaaattcatgtggtttctatgcaaagttcttcatatcatcacaacattatttgatttaaataaaattgaaagtaatatttgtgcaa

SEQ ID NO: 5 - RNA, 19 nt, Artificial Sequence
  Synthetic Construct
ggauuguguggaauguaag

SEQ ID NO: 6 - RNA, 19 nt, Artificial Sequence
  Synthetic Construct
cuuacauuccacacaaucc